(19) United States
(12) Patent Application Publication
(10) Pub. No.: US 2004/0210056 A1
(43) Pub. Date: Oct. 21, 2004

(54) Water compatible sterically hindered alkoxyamines and hydroxy substituted alkoxyamines

(76) Inventors: Mervin G. Wood, Mobile, AL (US); Robert Detlefsen, Putnam Valley, NY (US); James Galbo, Wingdale, NY (US); Wanda Martin, Fruitdale, AL (US); Paul Kondracki, Mobile, AL (US); Michael P. DiFazio, Mobile, AL (US); Joseph E. Babiarz, Amawalk, NY (US)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/782,524

(22) Filed: Feb. 19, 2004

Related U.S. Application Data
(60) Provisional application No. 60/450,262, filed on Feb. 26, 2003.

(51) Int. Cl. C07D 211/54; C07D 211/92

(52) U.S. Cl. 546/216; 546/223

(57) ABSTRACT

Sterically hindered alkoxyamine and hydroxy substituted alkoxyamine stabilizer compounds are made water compatible via certain backbones with affinity towards water. The sterically hindered amines are for example of the formula

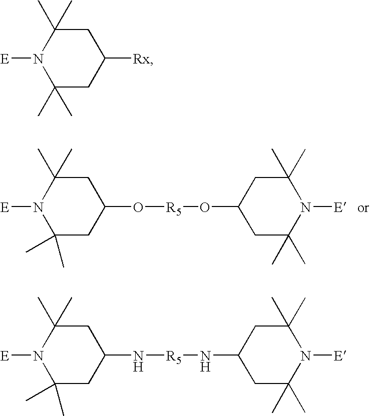

where for example E and E′ are 2-hydroxycyclohexyloxy, 2-hydroxy-2-methylpropoxy, benzyloxy, methoxy, propoxy, hexyloxy, heptyloxy, octyloxy or cyclohexyloxy, Rₓ is for example —NH₂⁺CH₂CH₂OH Cl⁻, —NH₃⁺⁻OAc, ═NOH, —NHCH(CH₃)COO⁻K⁺, —NHCH₂CH₂N(CH₃)₂⁺⁻OAc, —NHCH₂CH₂SO₃^{-K}⁺, —NHCH(COO⁻K⁺)CH₂CH₂SCH₃, —NHCH₂COO⁻K⁺, —OCH(CH₃)COO⁻K⁺, —OCH₂CH₂N(CH₃)₂⁺⁻OAc, —OCH₂CH₂SO₃⁻K⁺, —OCH(COO⁻K⁺)CH₂CH₂SCH₃ or —OCH₂COO⁻K⁺, and where R₅ comprises repeating units of —(OCH₂CH₂)—, —(OCH₂CH₂(CH₃))—, —(CH₂CHCOOH)—, —(CH₂C(CH₃)COOH)—, —(CH₂CHCOOCH₃)—, —(NHCH₂CH₂)—, —(CH₂CHOH)—, —(CH₂CHCONH₂)— or —(CH₂CH(NHCOH))—. These compounds are particularly effective in stabilizing aqueous polymer systems against the deleterious effects of oxidative, thermal and actinic radiation. The compounds are effective for example in stabilizing water borne coatings, aqueous inks, aqueous ink jet media and photocured aqueous systems.

This application claims the benefit under 35 USC 119(e) of U.S. patent app. No. 60/450,262, filed Feb. 26, 2003.

The instant invention pertains to sterically hindered alkoxyamine and hydroxy substituted alkoxyamine stabilizer compounds which are water compatible via certain backbones with affinity towards water. These materials are particularly effective in stabilizing aqueous polymer systems against the deleterious effects of oxidative, thermal and actinic radiation. The compounds are effective for example in stabilizing water borne coatings, aqueous inks, aqueous ink jet media and photocured aqueous systems.

U.S. Pat. Nos. 5,004,770 and 5,096,950 describe hindered amine compounds which are substituted on the N-atom by alkoxy moieties.

U.S. Pat. Nos. 6,271,377, 6,392,041 and 6,376,584 disclose sterically hindered hydroxy substituted alkoxyamines.

U.S. Pat. No. 6,254,724 teaches hindered hydroxylamine salt compounds.

U.S. Pat. No. 6,465,645 discloses long chain hindered amine stabilizers.

U.S. Pat. Nos. 5,286,865 and 5,216,156 disclose non-migrating hindered amine stabilizers.

U.S. published app. No. 2002/0050226 and equivalent EP 1174476 disclose certain hindered amines with certain water soluble groups.

U.S. Pat. No. 6,102,997 discloses certain hindered amine compounds with water solubilizing groups.

JP2000044851 teaches an ink composition that contains certain hindered amine compounds.

JP99170686 teaches hindered amine type additives generically in the ink receiving layer of ink jet recording media.

The present compounds are sterically hindered alkoxyamines and sterically hindered hydroxy substituted alkoxyamines, which are made water compatible or water soluble with a water compatible or water soluble backbone.

The instant compounds perform extremely well in aqueous polymeric systems and polar high solids systems. The present compounds exhibit excellent compatibility in polar environments such as polyurethane based coating systems, water borne automotive coating systems, polar recording media and aqueous inks.

### Detailed Disclosure

The present water compatible or water soluble sterically hindered alkoxyamines and hydroxy substituted alkoxyamines are of the formula (1)-(10)

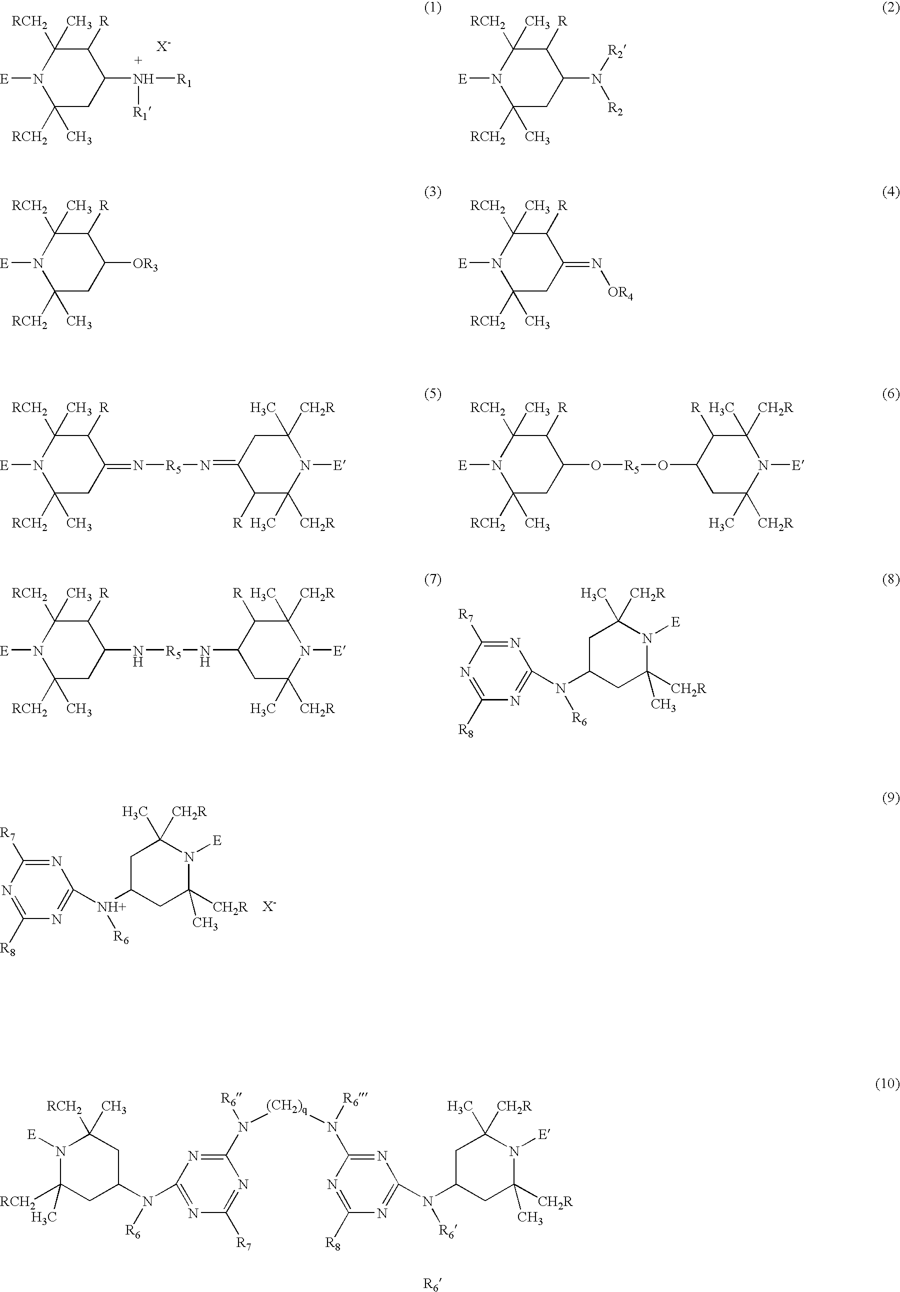

where

E is alkoxy of 1 to 18 carbon atoms, cycloalkoxy of 5 to 12 carbon atoms or aralkoxy of 7 to 15 carbon atoms, or E is -O-T-(OH)_{b},

T is a straight or branched chain alkylene of 1 to 18 carbon atoms, cycloalkylene of 5 to 18 carbon atoms, cycloalkenylene of 5 to 18 carbon atoms, a straight or branched chain alkylene of 1 to 4 carbon atoms substituted by phenyl or by phenyl substituted by one or two alkyl groups of 1 to 4 carbon atoms;

b is 1, 2 or 3 with the proviso that b cannot exceed the number of carbon atoms in T, and when b is 2 or 3, each hydroxyl group is attached to a different carbon atoms of T;

E′ is hydrogen, C₁-C₁₈alkyl, C₂-C₁₈alkenyl, C₇-C₁₅phenylalkyl, C₂-C₁₈alkanoyl or phenyl, or E′ is independently defined as for E,

R is hydrogen or methyl,

R₁ is hydrogen, C₁-C₁₂alkyl, C₅-C₈cycloalkyl, C₅-C₈cycloalkyl substituted by one to three C₁-C₄alkyl, C₂-C₁₂alkenyl, phenyl, C₇-C₉phenylalkyl, glycidyl, C₂-C₁₂alkanoyl, C₆-C₉cycloalkylcarbonyl, C₂-C₁₂carbamoyl, C₂-C₁₂alkenoyl, benzoyl, benzoyl substituted by one to three C₁-C₄alkyl, C₂-C₁₂alkanoyl substituted by a di(C₁-C₆alkyl) phosphonate,

or R₁ is C₂-C₁₂alkyl, C₂-C₁₂alkanoyl or C₇-C₁₈phenylalkyl, each interrupted by one to six oxygen, sulfur or —N(R₆)— groups; C₁-C₁₂alkyl, C₂-C₁₂alkanoyl, phenyl or C₇-C₁₈phenylalkyl, each substituted by one to six hydroxy groups or by one to six —NHR₆ groups; C₂-C₁₂alkyl, C₂-C₁₂alkanoyl or C₇-C₁₈phenylalkyl, each interrupted by one to three —NR₆C(O)— groups; or C₁-C₁₂alkyl, C₂-C₁₂alkanoyl, phenyl or C₇-C₁₈phenylalkyl, each substituted by one to three —SO₃H groups or by one to three —COOR₆ groups; or

R₁ is said alkyl substituted by a piperazine or by a morpholine group; or

R₁ is said interrupted group further substituted by one to six hydroxy groups or by one to six —NHR₆ groups; or

R₁ is said interrupted group further substituted by one to three —SO₃H groups or by one to three —COOR₆ groups;

or R₁ is a mono-valent homo- or co-oligomer consisting of monomer units derived from monomers selected from the group consisting of ethylene oxide, propylene oxide, ethylene glycol, propylene glycol, acrylic acid, methacrylic acid, ethylene imine, acrylamide, vinyl formamide, vinyl alcohol and vinyl acetate; which homo- or co-oligomer consists of between 2 and about 24 monomer units;

R₁′ is independently defined as for R₁,

R₅ is a divalent homo- or co-oligomer consisting of monomer units derived from monomers selected from the group consisting of ethylene oxide, propylene oxide, ethylene glycol, propylene glycol, acrylic acid, methacrylic acid, ethylene imine, acrylamide, vinyl formamide, vinyl alcohol and vinyl acetate; which homo- or co-oligomer consists of between 2 and about 24 monomer units,

R₆ is hydrogen or C₁-C₆alkyl,

R₆′, R₆″ and R₆″′ are independently defined as for R₆,

R₇ is —N(R₂)(R₂′) or is chlorine, alkoxy of 1 to 12 carbon atoms, 2-hydroxyethylamino or —N(R₆)(R₆′);

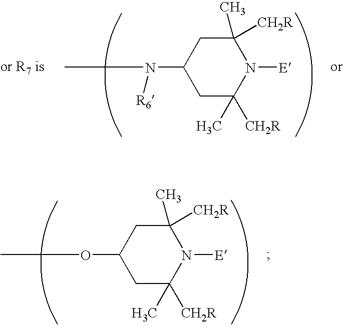

R₈ is defined as for R₇, where one of R₇ and R₈ is —N(R₂)(R₂′);

q is 2 to 8;

X⁻ is an inorganic or organic anion,

Y⁺ is a mono-, di- or tri-valent cation, and

when E is is -O-T-(OH)_{b},

R₂ is glycidyl, C₂-C₁₂alkanoyl substituted by a di(C₁-C₆alkyl) phosphonate, or

R₂ is C₂-C₁₂alkyl, C₂-C₁₂alkanoyl or C₇-C₁₈phenylalkyl, each interrupted by one to six oxygen, sulfur or —N(R₆)— groups; C₁-C₁₂alkyl, C₂-C₁₂alkanoyl, phenyl or C₇-C₁₈phenylalkyl, each substituted by one to six hydroxy groups or by one to six —NHR₆ groups; C₂-C₁₂alkyl, C₂-C₁₂alkanoyl or C₇-C₁₈phenylalkyl, each interrupted by one to three —NR₆C(O)— groups; or R₂ is C₁-C₁₂alkyl, C₂-C₁₂alkanoyl, phenyl or C₇-C₁₈phenylalkyl, each substituted by one to three —SO₃H groups or by one to three —COOR₆ groups; or

R₂ is said alkyl substituted by a piperazine or by a morpholine group; or

R₂ is said interrupted group further substituted by one to six hydroxy groups or by one to six —NHR₆ groups; or

R₂ is said interrupted group further substituted by one to three —SO₃H groups or by one to three —COOR₆ groups; or

R₂ is C₁-C₁₂alkyl, C₂-C₁₂alkanoyl, phenyl or C₇-C₁₈phenylalkyl, each substituted by one or two —COO⁻Y⁺, —N(R₆)(R₆′)⁺X⁻ or —SO₃⁻Y⁺ groups; or

R₂ is said C₁-C₁₂alkyl, C₂-C₁₂alkanoyl, phenyl or C₇-C₁₈phenylalkyl, each of which is substituted by one or two —COO⁻Y⁺, —N(R₆)(R₆′)⁺X⁻ or —SO₃⁻Y⁺ groups, each further substituted by one or two —OH, —COOR₆ or —NHR₆ groups; or

R₂ is a mono-valent homo- or co-oligomer consisting of monomer units derived from monomers selected from the group consisting of ethylene oxide, propylene oxide, ethylene glycol, propylene glycol, acrylic acid, methacrylic acid, ethylene imine, acrylamide, vinyl formamide, vinyl alcohol and vinyl acetate; which homo- or co-oligomer consists of between 2 and about 24 monomer units,

R₂′ is defined as for R₂ and may also be hydrogen,

R₃ is defined as for R₂ and may also be —SO₃H, —PO₃H₂, —SO₃⁻Y⁺ or —PO₃H⁻Y⁺, and

R₄ is defined as for R₂ and may also be hydrogen,

and

when E is alkoxy of 1 to 18 carbon atoms, cycloalkoxy of 5 to 12 carbon atoms or aralkoxy of 7 to 15 carbon atoms,

R₂ is C₁-C₁₂alkyl, C₂-C₁₂alkanoyl, phenyl or C₇-C₁₈phenylalkyl, each substituted by one or two —COO⁻Y⁺, —N(R₆)(R₆′)⁺X⁻ or —SO₃⁻Y⁺ groups; or

R₂ is said C₁-C₁₂alkyl, C₂-C₁₂alkanoyl, phenyl or C₇-C₁₈phenylalkyl, each of which is substituted by one or two —COO⁻Y⁺, —N(R₆)(R₆′)⁺X⁻ or —SO₃⁻Y⁺ groups, each further substituted by one or two —OH, —COOR₆ or —NHR₆ groups, with the proviso that the compound

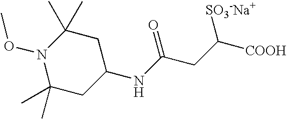

is not included; or

R₂ is a mono-valent homo- or co-oligomer consisting of monomer units derived from monomers selected from the group consisting of ethylene oxide, propylene oxide, ethylene glycol, propylene glycol, acrylic acid, methacrylic acid, ethylene imine, acrylamide, vinyl formamide, vinyl alcohol and vinyl acetate; which homo- or co-oligomer consists of between 2 and about 24 monomer units;

R₂′ is defined as for R₂ and may also be hydrogen,

R₃ is defined as for R₂ and may also be —SO₃H, —PO₃H₂, —SO₃⁻Y⁺ or —PO₃H⁻Y⁺, and

R₄ is defined as for R₂ and may also be hydrogen.

For example, E is -O-T(OH)_{b}.

For example, E is 2-hydroxycyclohexyloxy or 2-hydroxy-2-methylpropoxy.

For example, the present compounds are of the formula

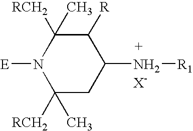

where

E is -O-T(OH)_{b},

R₁ is hydrogen, C₁-C₆alkyl, C₂-C₆alkanoyl, C₂-C₆alkyl or C₂-C₆alkanoyl interrupted by one or two oxygen, sulfur or —N(R₆)— groups; C₁-C₆alkyl or C₂-C₆alkanoyl substituted by one to three hydroxy groups or by one to three —NHR₆ groups, C₂-C₆alkyl or C₂-C₆alkanoyl interrupted by a —NR₆C(O)— group, or is C₁-C₆alkyl or C₂-C₆alkanoyl substituted by a —SO₃H or by a —COOR₆ group, and

the other moities are as previously described.

For example, the present compounds are of the formula

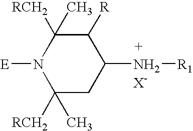

where

E is -O-T(OH)_{b},

R₁ is hydrogen, C₁-C₄alkyl, C₂-C₅alkanoyl, C₂-C₄alkyl or C₂-C₅alkanoyl interrupted by an oxygen, sulfur or —N(R₆)— group; C₁-C₄alkyl or C₂-C₅alkanoyl substituted by an hydroxy group or by a —NHR₆ group, C₂-C₄alkyl or C₂-C₅alkanoyl interrupted by a —NR₆C(O)— group, or is C₁-C₄alkyl or C₂-C₅alkanoyl substituted by a —SO₃H or by a —COOR₆ group,

and the other moieties are as described previously.

For instance, the present compounds are of the formula

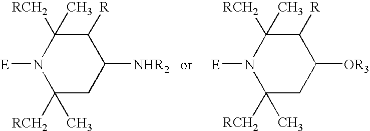

where

E is -O-T(OH)_{b},

R₂ and R₃ are C₂-C₆alkyl or C₂-C₆alkanoyl interrupted by one or two oxygen, sulfur or —N(R₆)— groups; C₁-C₆alkyl or C₂-C₆alkanoyl substituted by one to three hydroxy groups or by one to three —NHR₆ groups, C₂-C₆alkyl or C₂-C₆alkanoyl interrupted by a —NR₆C(O)— group, or R₂ is C₁-C₆alkyl or C₂-C₆alkanoyl substituted by a —SO₃H group or by a —COOR₆ group; or

R₂ and R₃ are C₁-C₆alkyl, C₂-C₆alkanoyl or C₇-C₉phenylalkyl, each substituted by a —COO⁻Y₊, —N(R₆)(R₆′)⁺X⁻ or —SO₃⁻Y⁺ group, and

the other moieties are as described previously.

For instance, the present compounds are of the formula

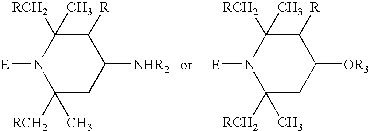

where

E is -O-T(OH)_{b},

R₂ and R₃ are C₂-C₄alkyl or C₂-C₅alkanoyl interrupted by an oxygen, sulfur or —N(R₆)— group; C₁-C₄alkyl or C₂-C₅alkanoyl substituted by an hydroxy group or by a —NHR₆ group, C₂-C₄alkyl or C₂-C₅alkanoyl interrupted by a —NR₆C(O)— group, or R₂ is C₁-C₄alkyl or C₂-C₅alkanoyl substituted by a —SO₃H group or by a —COOR₆ group; or

R₂ and R₃ are C₁-C₄alkyl or C₂-C₅alkanoyl substituted by a —COO⁻Y⁺, —N(R₆)(R₆′)⁺X⁻ or —SO₃⁻Y⁺ group, and

the other moieties are as described previously.

For example, the present compounds are of the formula

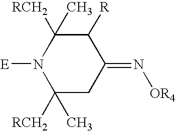

where

E is -O-T(OH)_{b},

R₄ is hydrogen, C₂-C₆alkyl or C₂-C₆alkanoyl interrupted by one or two oxygen, sulfur or —N(R₆)— groups; C₁-C₆alkyl or C₂-C₆alkanoyl substituted by one to three hydroxy groups or by one to three —NHR₆ groups, C₂-C₆alkyl or C₂-C₆alkanoyl interrupted by a —NR₆C(O)— group, or R₄ is C₁-C₆alkyl or C₂-C₆alkanoyl substituted by a —SO₃H group or by a —COOR₆ group; or

R₄ is C₁-C₆alkyl, C₂-C₆alkanoyl or C₇-C₉phenylalkyl, each substituted by a —COO⁻Y⁺, —N(R₆)(R₆′)⁺X⁻ or —SO₃⁻Y⁺ group, and

the other moieties are as described previously.

For example, the present compounds are of the formula

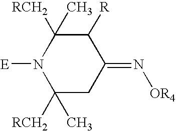

where

E is -O-T(OH)_{b},

R₄ is hydrogen, C₂-C₄alkyl or C₂-C₅alkanoyl interrupted by an oxygen, sulfur or —N(R₆)— group; C₁-C₄alkyl or C₂-C₅alkanoyl substituted by an hydroxy group or by a —NHR₆ group, C₂-C₄alkyl or C₂-C₅alkanoyl interrupted by a —NR₆C(O)— group, or R₄ is C₁-C₄alkyl or C₂-C₅alkanoyl substituted by a —SO₃H group or by a —COOR₆ group; or

R₄ is C₁-C₄alkyl or C₂-C₅alkanoyl substituted by a —COO⁻Y⁺, —N(R₆)(R₆′)⁺X⁻ or —SO₃⁻Y⁺ group, and

the other moities are as described previously.

For instance, the present compounds are of the formula

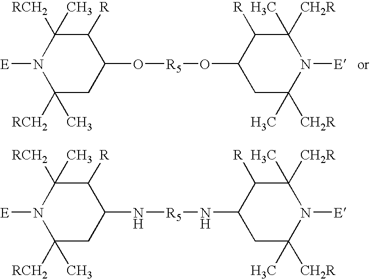

where

E is -O-T(OH)_{b},

R₅ is polyethylene glycol or polypropylene glycol, and

the other moities are as described previously.

For example, the present compounds are of the formula

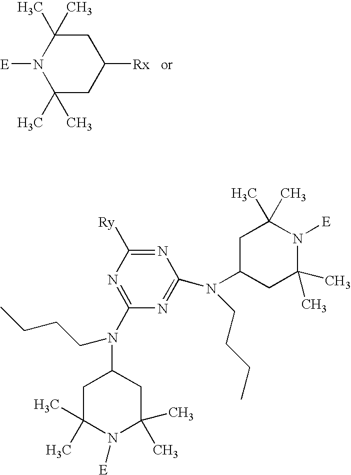

where

E is -O-T(OH)_{b}, and

where Rₓ is selected from the group consisting of

—NH₂⁺CH₂CH₂OH Cl⁻, —NHCH₂CH₂OH, —NH₃⁺⁻OAc, ═NOH, −NHCH(CH₃)COO⁻K⁺, —NHCH₂CH₂N(CH₃)₂⁺⁻OAc, —NHCH₂CH₂SO₃⁻K⁺, —NHCH(COO⁻K⁺)CH₂CH₂SCH₃, —NHCH₂COO⁻K⁺, —NHCOCH₂OH, —NHCOCH₂NHCOCH₃, —NHCH₂CH₂CH₂SO₃H, —OCH₂CH₂OH, —OCH(CH₃)COO⁻K⁺, —OCH₂CH₂N(CH₃)₂⁺⁻OAc, —OCH₂CH₂SO₃⁻K⁺, —OCH(COO⁻K⁺)CH₂CH₂SCH₃, —OCH₂COO⁻K⁺, —OCOCH₂OH, —OCOCH₂NHCOCH₃ and —OCH₂CH₂CH₂SO₃H; and

where R_{y} is selected from the group consisting of

—NHCH₂CH₂NHCH₂CH₂NHCH₂CH₂NH₂, —NH₂⁺CH₂CH₂NHCH₂CH₂NHCH₂CH₂NH₂⁻OAc, —NHPhSO₃H, —NHPhSO₃⁻K⁺, —NHPhSO₃⁻Na⁺, —NH₂⁺PhSO₃H Cl⁻, —NH(3-carboxy-4-chlorophenyl), —NH(3-COO⁻Na⁺-4-chlorophenyl), —NHCH₂CH₂—(N-piperazine), —NH₂⁺CH₂CH₂—(N-piperazine) ⁻OAc and —NH₂⁺CH₂CH₂—(N-piperazine) ⁻Cl.

For example, E is alkoxy of 1 to 18 carbon atoms, cycloalkoxy of 5 to 12 carbon atoms or aralkoxy of 7 to 15 carbon atoms.

For instance, E is benzyloxy, methoxy, propoxy, butoxy, pentoxy, hexyloxy, heptyloxy, octyloxy or cyclohexyloxy.

For example, the present compounds are of the formula

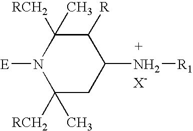

where

E is alkoxy of 1 to 18 carbon atoms, cycloalkoxy of 5 to 12 carbon atoms or aralkoxy of 7 to 15 carbon atoms,

R₁ is hydrogen, C₁-C₆alkyl, C₂-C₆alkanoyl, C₂-C₆alkyl or C₂-C₆alkanoyl interrupted by one or two oxygen, sulfur or —N(R₆)— groups; C₁-C₆alkyl or C₂-C₆alkanoyl substituted by one to three hydroxy groups or by one to three —NHR₆ groups, C₂-C₆alkyl or C₂-C₆alkanoyl interrupted by a —NR₆C(O)— group, or is C₁-C₆alkyl or C₂-C₆alkanoyl substituted by a —SO₃H or by a —COOR₆ group, and

the other moieties are as described previously.

For example, the present compounds are of the formula

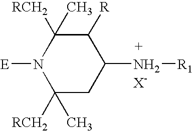

where

E is alkoxy of 1 to 18 carbon atoms, cycloalkoxy of 5 to 12 carbon atoms or aralkoxy of 7 to 15 carbon atoms,

R₁ is hydrogen, C₁-C₄alkyl, C₂-C₅alkanoyl, C₂-C₄alkyl or C₂-C₅alkanoyl interrupted by an oxygen, sulfur or —N(R₆)— group; C₁-C₄alkyl or C₂-C₅alkanoyl substituted by an hydroxy group or by a —NHR₆ group, C₂-C₄alkyl or C₂-C₅alkanoyl interrupted by a —NR₆C(O)— group, or is C₁-C₄alkyl or C₂-C₅alkanoyl substituted by a —SO₃H or by a —COOR₆ group, and

the other moities are as described previously.

For instance, the present compounds are of the formula

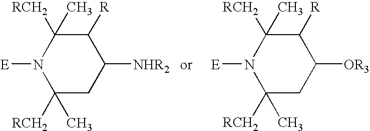

where

E is alkoxy of 1 to 18 carbon atoms, cycloalkoxy of 5 to 12 carbon atoms or aralkoxy of 7 to 15 carbon atoms,

R₂ and R₃ are C₁-C₆alkyl, C₂-C₆alkanoyl or C₇-C₉phenylalkyl, each substituted by a —COO⁻Y⁺, —N(R₆)(R₆′)⁺X⁻ or —SO₃⁻Y⁺ group, and

the other moieties are as described previously.

For instance, the present compounds are of the formula

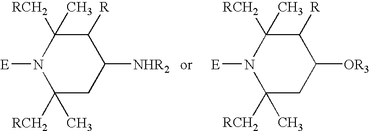

where

E is alkoxy of 1 to 18 carbon atoms, cycloalkoxy of 5 to 12 carbon atoms or aralkoxy of 7 to 15 carbon atoms,

R₂ and R₃ are C₁-C₄alkyl or C₂-C₅alkanoyl substituted by a —COO⁻Y⁺, —N(R₆)(R₆′)⁺X⁻ or —SO₃⁻Y⁺ group, and

the other moities are as described previously.

For example, the present compounds are of the formula

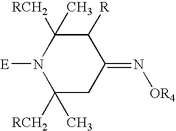

where

E is alkoxy of 1 to 18 carbon atoms, cycloalkoxy of 5 to 12 carbon atoms or aralkoxy of 7 to 15 carbon atoms,

R₄ is C₁-C₆alkyl, C₂-C₆alkanoyl or C₇-C₉phenylalkyl, each substituted by a —COO⁻Y⁺, —N(R₆)(R₆′)⁺X⁻ or —SO₃⁻Y⁺ group, and

the other moities are as described previously.

For example, the present compounds are of the formula

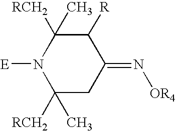

where

E is alkoxy of 1 to 18 carbon atoms, cycloalkoxy of 5 to 12 carbon atoms or aralkoxy of 7 to 15 carbon atoms,

R₄ is C₁-C₄alkyl or C₂-C₅alkanoyl substituted by a —COO⁻Y⁺, —N(R₆)(R₆′)⁺X⁻ or —SO₃⁻Y⁺ group, and

the other moities are as described previously.

For instance, the present compounds are of the formula

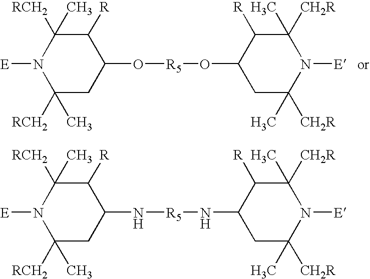

where

E is alkoxy of 1 to 18 carbon atoms, cycloalkoxy of 5 to 12 carbon atoms or aralkoxy of 7 to 15 carbon atoms,

R₅ is polyethylene glycol or polypropylene glycol, and

the other moities are as described previously.

For example the present compounds are of the formula

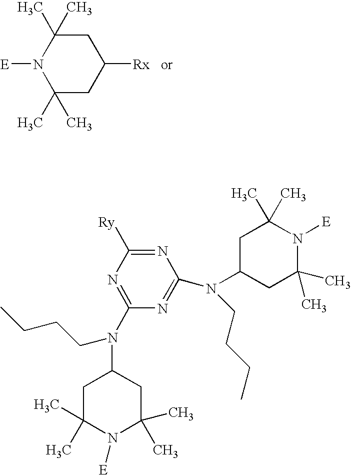

where

E is alkoxy of 1 to 18 carbon atoms, cycloalkoxy of 5 to 12 carbon atoms or aralkoxy of 7 to 15 carbon atoms, and

Rₓ is selected from the group consisting of

—NH₂⁺CH₂CH₂OH Cl⁻, —NH₃⁺⁻OAc, ═NOH, —NHCH(CH₃)COO⁻K⁺, —NHCH₂CH₂N(CH₃)₂⁺⁻OAc, —NHCH₂CH₂SO₃⁻K⁺, —NHCH(COO⁻K⁺)CH₂CH₂SCH₃, —NHCH₂COO⁻K⁺, —OCH(CH₃)COO⁻K⁺, —OCH₂CH₂N(CH₃)₂⁺⁻OAc, —OCH₂CH₂SO₃⁻K⁺, —OCH(COO⁻K⁺)CH₂CH₂SCH₃ and —OCH₂COO⁻K⁺ and

where R_{y} is selected from the group consisting of

—NHCH₂CH₂NHCH₂CH₂NHCH₂CH₂NH₂, —NH₂⁺CH₂CH₂NHCH₂CH₂NHCH₂CH₂NH₂⁻OAc, —NHPhSO₃H, —NHPhSO₃⁻K⁺, —NHPhSO₃⁻Na⁺, —NH₂⁺PhSO₃H Cl⁻, —NH(3-carboxy-4-chlorophenyl), —NH(3-COO⁻Na⁺-4-chlorophenyl), —NHCH₂CH₂—(N-piperazine), —NH₂⁺CH₂CH₂—(N-piperazine) ⁻OAc and —NH₂⁺CH₂CH₂—(N-piperazine) ⁻Cl.

The solubility of the present sterically hindered amines in water at 20° C. and standard pressure is for example greater than or equal to 1 g/L, for example ≧2 g/L, ≧5 g/L, ≧10 g/L, ≧20 g/L, ≧30 g/L, ≧40 g/L, ≧50 g/L, ≧60 g/L, ≧70 g/L, ≧80 g/L, ≧90 g/L or ≧100 g/L.

Alkyl having up to 12 carbon atoms is branched or unbranched, and is for example for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl and dodecyl, 1,1,3,3,5,5-hexamethylhexyl.

Alkenyl is an unsaturated version of alkyl, and is branched or unbranched, for example isopropenyl, propenyl, hexenyl, heptenyl, and the like.

Unsubstituted or C₁-C₄alkyl-substituted C₅-C₈cycloalkyl is, for example, cyclopentyl, methylcyclopentyl, dimethylcyclopentyl, cyclohexyl, methylcyclohexyl, dimethylcyclohexyl, trimethylcyclohexyl, tert-butylcyclohexyl, cycloheptyl or cyclooctyl. For example cyclohexyl and tert-butylcyclohexyl.

C₁-C₄Alkyl-substituted phenyl, which contains for example 1 to 3, for instance 1 or 2, alkyl groups, is, for example, o-, m- or p-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2-methyl-6-ethylphenyl, 4-tert-butylphenyl, 2-ethylphenyl or 2,6-diethylphenyl.

Phenylalkyl includes substituted phenylalkyl, for example phenylalkyl substituted on the phenyl ring by from 1 to 3 C₁-C₄alkyl groups or from 1 to 3 halogen or by a mixture thereof, and is for example, benzyl, 4-chlorobenzyl, α-methylbenzyl, α,α-dimethylbenzyl, 2-phenylethyl, 2-methylbenzyl, 3-methylbenzyl, 4-methylbenzyl, 2,4-dimethylbenzyl, 2,6-dimethylbenzyl or 4-tert-butylbenzyl.

Phenyl includes unsubstituted phenyl and phenyl substituted by from 1 to 3 C₁-C₄ alkyl groups or from 1 to 3 halogen or by a mixture thereof.

Alkanoyl having up to 12 carbon atoms is branched or unbranched, and is for example formyl, acetyl, propionyl, butanoyl, pentanoyl, hexanoyl, heptanoyl, octanoyl, nonanoyl, decanoyl, undecanoyl or dodecanoyl.

C₆-C₉Cycloalkylcarbonyl is, for example, cyclopentylcarbonyl, cyclohexylcarbonyl, cycloheptylcarbonyl or cyclooctylcarbonyl.

Carbamoyl of up to 12 carbon atoms is branched or unbranched, and is for example the carbamoyl equivalent of the alkanoyl groups, for example butamoyl, pentamoyl, hexamoyl and the like.

Alkenoyl of up to 12 carbon atoms is branched or unbranched, and is an unsaturated version of alkanoyl.

Benzoyl substituted by one to three C₁-C₄alkyl, is for example o-, m- or p-methylbenzoyl, 2,3-dimethylbenzoyl, 2,4-dimethylbenzoyl, 2,5-dimethylbenzoyl, 2,6-dimethylbenzoyl, 3,4-dimethylbenzoyl, 3,5-dimethylbenzoyl, 2-methyl-6-ethylbenzoyl, 4-tert-butylbenzoyl, 2-ethylbenzoyl, 2,4,6-trimethylbenzoyl, 2,6-dimethyl-4-tert-butylbenzoyl or 3,5-di-tert-butylbenzoyl.

C₂-C₂₅Alkanoyl substituted by a di(C₁-C₆alkyl) phosphonate group is, for example, (CH₃CH₂O)₂POCH₂CO—, (CH₃O)₂POCH₂CO—, (CH₃CH₂CH₂CH₂O)₂POCH₂CO—, (CH₃CH₂O)₂POCH₂CH₂CO—, (CH₃O)₂POCH₂CH₂CO—, (CH₃CH₂CH₂CH₂O)₂POCH₂CH₂CO—, (CH₃CH₂O)₂PO(CH₂)₄CO—, (CH₃CH₂O)₂PO(CH₂)₈CO— or (CH₃CH₂O)₂PO(CH₂)₁₇CO—.

C₂-C₁₂Alkyl interrupted by oxygen, sulfur or by —N(R₆)— is, for example, CH₃—O—CH₂—, CH₃—S—CH₂—, CH₃—N(CH₃)—CH₂—, CH₃—O—CH₂CH₂—O—CH₂—, CH₃—(O—CH₂CH₂—)₂O—CH₂—, CH₃—(O—CH₂CH₂—)₃O—CH₂— or CH₃—(O—CH₂CH₂—)₄O—CH₂—.

C₃-C₁₂Alkanoyl interrupted by oxygen, sulfur or by —N(R₆)— is, for example, CH₃—O—CH₂CO—, CH₃—S—CH₂CO—, CH₃—N(CH₃)—CH₂CO—, CH₃—O—CH₂CH₂—O—CH₂CO—, CH₃—(O—CH₂CH₂—)₂O—CH₂CO—, CH₃—(O—CH₂CH₂—)₃O—CH₂CO— or CH₃—(O—CH₂CH₂—)₄O—CH₂CO—.

Substitution by one to six also means of course by 1, 2, 3, 4, 5 or 6 groups, if the length of the chain so allows. Substitution by one to three means of course by 1, 2 or 3 groups, if the length of the chain allows.

Interruption by one to six groups means of course by 1, 2, 3, 4, 5 or 6 groups, if length of the chain allows.

C₇-C₁₈Phenylalkyl interrupted by oxygen, sulfur or by —N(R₆)— and unsubstituted or substituted on the phenyl radical by from one to three C₁-C₄alkyl groups is branched or unbranched, and is for example phenoxymethyl, 2-methyl-phenoxymethyl, 3-methyl-phenoxymethyl, 4-methyl-phenoxymethyl, 2,4-dimethyl-phenoxymethyl, 2,3-dimethyl-phenoxymethyl, phenylthiomethyl, N-methyl-N-phenyl-aminomethyl, N-ethyl-N-phenyl-aminomethyl, 4-tert-butyl-phenoxymethyl, 4-tert-butyl-phenoxyethoxy-methyl, 2,4-di-tert-butyl-phenoxymethyl, 2,4-di-tert-butyl-phenoxyethoxymethyl, phenoxyethoxyethoxyethoxymethyl, benzyloxymethyl, benzyloxyethoxymethyl, N-benzyl-N-ethyl-aminomethyl or N-benzyl-N-isopropyl-aminomethyl.

The oligomers and co-oligomers of ethylene oxide, propylene oxide, ethylene glycol, propylene glycol, acrylic acid, methacrylic acid, ethylene imine, vinyl alcohol and vinyl acetate are of course oligomeric and co-oligomeric versions of poly(ethylene oxide), poly(propylene oxide), polyethylene glycol, polypropylene glycol, polyacrylic acid, polymethacrylic acid, poly(ethylene imine), polyacrylamide, polyvinylformamide, polyvinyl alcohol and polyvinyl acetate. Oligomers of the above, when mono-valent, may be capped with for example a methyl group or an acetate group, for example the oligomers of polyethylene glycol and polypropylene glycol. Homo- or co-oligomers between 2 and about 24 monomer units are for example between 2 and 20 monomer units, between 2 and 17 monomer units, between 2 and 14 monomer units, between 2 and 11 monomer units, between 2 and 9 monomer units, between 2 and 8 monomer units, between 2 and 6 monomer units, between 2 and 5 monomer units, or between 2 and 4 monomer units. The total number of carbon atoms in the oligomers and co-oligomers is for example less than 20.

X⁻ is an inorganic or organic anion, such as phosphate, phosphonate, carbonate, bicarbonate, nitrate, chloride, bromide, bisulfite, sulfite, bisulfate, sulfate, borate, formate, acetate (OAc), benzoate, citrate, oxalate, tartrate, acrylate, polyacrylate, fumarate, maleate, itaconate, glycolate, gluconate, malate, mandelate, tiglate, ascorbate, polymethacrylate, a carboxylate of nitrilotriacetic acid, hydroxyethylethylenediaminetriacetic acid, ethylenediaminetetraacetic acid or of diethylenetriaminepentaacetic acid, a diethylenetriaminepentamethylenephosphonate, an alkylsulfonate or an arylsulfonate. Of course, where it is for example a di-, tri- or quaternary valent anion, it forms ion pairs with 2, 3 or 4 cations respectively.

Y⁺ is a mono-, di- or tri-valent cation and is for example an alkali metal cation, alkaline earth metal cation or aluminum cation. For example, Y⁺ is Na⁺, K⁺, Mg⁺⁺, Ca⁺⁺ or Al⁺⁺⁺. Of course, where it is a di- or tri-cation, it forms ion pairs with 2 or 3 anions respectively.

Also subject of the present invention are stabilized compositions comprising

an organic material subject to the deleterious effects of light, heat and oxygen, and

an effective stabilizing amount of a present water compatible or water soluble sterically hindered alkoxyamine or hydroxy substituted alkoxyamine compound.

The present water compatible or water soluble sterically hindered alkoxyamines or hydroxy substituted alkoxyamine compounds are particularly effective towards preventing color fading of compositions comprising pigments or dyes.

Accordingly, colored compositions comprising pigments or dyes are stabilized compositions according to this invention. That is, the stabilized organic material is the pigment or dye.

The colored compositions are for example compositions comprising dyes, which compositions are selected from the group consisting of ink jet inks, ink jet recording media, coatings, body care products, household products, textiles and fabrics.

The body care products, household products, textiles and fabrics are as described in U.S. application Ser. No. 60/377,381, filed May 2, 2002, published as WO 03/103622, the disclosure of which is hereby incorporated by reference.

The body care products are for example hair care products such as shampoos or hair dyeing agents or dentrifices such as mouthwashes.

The present stabilized compositions are for example automotive coating compositions. Compositions to be stabilized including automotive coating compositions are disclosed for example in U.S. Pat. Nos. 5,977,219 and 6,166,218, the disclosures of which are hereby incorporated by reference.

The acrylic resin lacquers which can be stabilized against light, moisture and oxygen in accordance to the instant invention are for example acrylic resin stoving lacquers or thermosetting resins including acrylic/melamine systems which are described, for example, in H. Kittel's “Lehrbuch and Beschichtungen”, Vol. 1 , Part 2 on pages 735 and 742 (Berlin 1972), “Lackkunstharze” (1977) by H. Wagner and H. F. Sarx on pages 229-238, and in S. Paul's “Surface Coatings: Science and Technology”, (1985).

The polyester lacquers which can be stabilized against the action of light and moisture are for instance stoving lacquers described e.g. in H. Wagner and H. F. Sarx, op. cit., on pages 86-99.

The alkyd resin lacquers which can be stabilized against the action of light and moisture in accordance with the instant invention are for example stoving lacquers which are used in particular for coating automobiles (automobile finishing lacquers), for example lacquers based on alkyd/melamine resins and alkyd/acrylic/melamine resins (see H. Wagner and H. F. Sarx, “Lackkunstharze” (1977), pages 99-123). Other crosslinking agents include glycoluril resins, blocked or unblocked isocyanates or epoxy resins. Other lacquers which can be stabilized include those with crosslinkable functionalities such as carbamate and siloxane.

The lacquers stabilized in accordance with the invention are suitable both for metal finish coatings and solid shade finishes, especially in the case of retouching finishes, as well as various coil coating applications. The lacquers stabilized in accordance with the invention are preferably applied in the conventional manner by two methods, either by the single-coat method or by the two-coat method. In the latter method, the pigment-containing base coat is applied first and then a covering coat of clear lacquer over it.

In addition to acid-catalyzed baked finishes, it is also to be noted that the compounds of the present invention are applicable for use in non-acid catalyzed thermoset resins such as epoxy, epoxy-polyester, vinyl, alkyd, acrylic and polyester resins, optionally modified with silicon, isocyanates or isocyanurates. The epoxy and epoxy-polyester resins are crosslinked with conventional crosslinkers such as acids, acid anhydrides, amines and the like. Correspondingly, the epoxide may be utilized as the crosslinking agent for various acrylic or polyester resin systems that have been modified by the presence of reactive groups on the backbone structure.

When used in two-coat finishes, the compounds of the instant invention can be incorporated in the clear coat or both in the clear coat and in the pigmented base coat.

To attain maximum light stability, the concurrent use of other conventional light stabilizers can be advantageous. Examples are UV (ultraviolet light) absorbers of the benzophenone, 2H-benzotriazole, acrylic acid derivatives, oxalanilide, aryl-s-triazine or metal-containing types (e.g. organic nickel compounds). In two-coat systems, these additional light stabilizers can be added to the clear coat and/or the pigmented base coat.

When water soluble, water miscible or water dispersible coatings are desired ammonium salts of acid groups present in the resin are formed. Powder coating compositions can be prepared by reacting glycidyl methacrylate with selected alcohol components.

The instant compounds are also useful in the stabilization of acid catalyzed thermoset resins which are disclosed in U.S. Pat. No. 5,112,890, the relevant parts of which are incorporated herein by reference.

These resins are used in baked enamels or stoving lacquers. Hindered amine light stabilizers are well known to be effective in stabilizing a host of organic substrates including polymers from the deleterious effects of oxygen and light. Such hindered amine light stabilizers have been used in the stabilization of hot-crosslinkable alkyd or acrylic metallic stoving lacquers (see U.S. Pat. No. 4,426,472) and in stabilizing acid-catalyzed stoving lacquers based on hot-crosslinkable acrylic polyester or alkyl resins (see U.S. Pat. Nos. 4,344,876 and 4,426,471). None of the hindered amine light stabilizers of these patents possess the present water-solubilizing structures. The instant compounds have such substitution.

In their industrial uses, enamels with high solids content based on crosslinkable acrylic, polyester, urethane or alkyd resins are cured with an additional acid catalyst. The present water compatible or water soluble sterically hindered alkoxyamines and hydroxy substituted alkoxyamines are suitable in such systems.

The acid catalyzed thermoset enamels must be stabilized in order to function acceptably in end-use applications. The stabilizers are needed to impart greater retention of durability to the cured enamels (as measured by 20° gloss, distinction of image, cracking or chalking); the stabilizers must not retard cure (normal bake for auto finishes at 121° C.; and low bake repair at 82° C.) as measured by hardness, adhesion, solvent resistance and humidity resistance; the enamel should not yellow on curing and further color change on exposure to light should be minimized.

The instant hindered amine light stabilizers fulfill each of these requirements and provide alone or in combination with a UV absorber outstanding light stabilization protection to the cured acid catalyzed thermoset enamels.

The instant invention also pertains to resin systems capable of being fully cured under ambient conditions. For example, applicable resins include alkyd, acrylic, polyester and epoxide resins as described in S. Paul's “Surface Coatings: Science and Technology” (1985), pages 70-310. Various acrylic and modified acrylic resins are described in H. Kittel's “Lehrbuch der Lacke unde Beschichtungen”, Vol. 1, Part 2, on pages 735 and 742 (Berlin 1972), and in “Lackkunstharze” (1977) by H. Wagner and H. F. Sarx, op. cit, on pages 229-238. Typical crosslinkable polyester resins which can be stabilized against the action of light and moisture are described e.g. in H. Wagner and H. F. Sarx, op. cit., on pages 86-99. The unmodified and modified alkyd resins which can be stabilized are conventional resins which are used in trade sales, maintenance and automotive refinish coatings. For example, such coatings are based on alkyd resins, alkyd/acrylic resins and alkyd/silicon reins (see H. Wagner and H. F. Sarx, op. cit., pages 99-123) optionally crosslinked by isocyanates or epoxy resins.

In addition various acrylic lacquer coating compositions are disclosed in U.S. Pat. No. 4,162,249. Other acrylic/alkyd resins with polyisocyanate additives are disclosed in U.S. Pat. No. 4,471,083; and acrylic resins containing either pendant amino ester groups or glycidyl groups are described in U.S. Pat. No. 4,525,521.

The ambient cured coatings stabilized by the instant compounds are suitable both for metal finish coatings and solid shade finishes, especially in the case of retouching finishes. The lacquers stabilized by the instant compounds are preferably applied in a conventional manner by two methods, either by the single-coat method or by the two-coat method. In the latter method, the pigment-containing base coat is applied first and a covering coat of clear lacquer applied over it. When used in two-coat finishes, the instant hindered amine compound can be incorporated in the clear coat or both in the clear coat and in the pigmented base coat.

The instant invention also pertains to abrasion-resistant coating compositions suitable for coating over polycarbonates. Such coatings as described in U.S. Pat. No. 5,214,085 comprise a silyl acrylate, aqueous colloidal silica, a photoinitiator and optionally a polyfunctional acrylate as well as UV absorbers. Such coatings provide resistance after prolonged outdoor exposure to sunlight, moisture, thermal cycling causing yellowing, delamination and formation of microcracks and decreasing transparency.

Related hindered amine stabilizers have been utilized individually and in combination with UV absorbers to improve the performance characteristics of ambient cured coating systems. Notwithstanding such improvements, there still exists a need to further retard the photooxidation and photodegradation of such ambient cured systems and thereby provide increased effectiveness by maintaining the physical integrity of the coatings. Such effectiveness can be manifested by prevention of embrittlement, cracking, corrosion, erosion, loss of gloss, chalking and yellowing of the coating.

It has now been determined that the aforementioned improvements can be achieved by the utilization of the present sterically hindered amines in ambient cured coating systems as is taught in U.S. Pat. No. 5,124,378, the relevant parts of which are incorporated herein by reference. In particular, the physical integrity of the coatings is maintained to a higher degree with significant reduction in loss of gloss and in yellowing. Accordingly, the instant invention relates to the use of the present sterically hindered alkoxyamines and hydroxy substituted alkoxyamines, optionally together with further stabilizers, for stabilizing ambient cured coatings based on alkyd resins; thermoset acrylic resins; acrylic alkyds; acrylic alkyd or polyester resins optionally modified with silicon, isocyanates, isocyanurates, ketimines or oxazolidines; and epoxide resins crosslinked with carboxylic acids, anhydrides, polyamines or mercaptans; and acrylic and polyester resin systems modified with reactive groups in the backbone thereof and crosslinked with epoxides; against the degradative effects of light, moisture and oxygen.

The instant invention also relates to electrodeposited coatings applied to metal substrates where various top coats may be applied thereover. The inclusion of the instant compounds in the E-coat provides delamination resistance to said E-coats. The primary resins in said E-coats are acrylic or epoxy resins. These E-coats are described in European patent application EP 0 576 943 A1.

The instant invention also pertains to water borne architectural coatings, for example, water borne latex emulsion paints.

The instant invention also pertains to ultraviolet light cured (UV cured) coating systems using unsaturated acrylic resins, polyurethane acrylates, epoxy acrylates, polyester acrylates, unsaturated polyester/styrene resins and silyl acrylates.

### Powder Coatings

The instant invention also pertains to powder coating formulations which require resistance to photodegradation. Resin systems which would be applicable include glycidyl methacrylate or acrylate-functional acrylic or acrylic hybrids, crosslinked with diacids or anhydrides; acid or anhydride functional acrylic or polyester resins crosslinked with TGIC; hydroxyl functional acrylic or polyester resins crosslinked with isocyanates. The stabilized coating may be a single layer applied to a substrate, or may be a clearcoat applied over a water borne or solvent borne basecoat.

The stabilized coating may also contain a UV absorber, consisting of one of the aforementioned compounds.

### Radiation-Cured Systems

The instant invention also pertains to radiation-cured coating systems. These systems comprise:

a. Ethylenically unsaturated polymerizable compounds

b. At least one photoinitiator

c. One or more of the instant stabilizing compounds

The coating composition may also include a UV absorbing stabilizer, represented by one of the classes mentioned.

The coating may also include pigments or other colorants designed to provide opacity or aesthetic properties.

The ethylenically unsaturated polymerizable compounds can contain one or more than one olefinic double bond. They may be low molecular (monomeric) or high molecular (oligomeric) compounds.

Typical examples of monomers containing one double bond are alkyl or hydroxyalkyl acrylates or methacrylates, for example methyl, ethyl, butyl, 2-ethylhexyl and 2-hydroxyethyl acrylate, isobornyl acrylate, and methyl and ethyl methacrylate. Further examples of these monomers are acrylonitrile, acrylamide, methacrylamide, N-substituted (meth)acrylamides, vinyl esters such as vinyl acetate, vinyl ethers such as isobutyl vinyl ether, styrene, alkylstyrenes, halostyrenes, N-vinylpyrrolidone, vinyl chloride and vinylidene chloride.

Examples of monomers containing more than one double bond are ethylene glycol diacrylate, propylene glycol diacrylate, neopentyl glycol diacrylate, hexamethylene glycol diacrylate, bisphenol A diacrylate, 4,4′-bis(2-acryloyloxyethoxy)diphenylpropane, trimethylolpropane triacrylate, pentaerythritol triacrylate and tetraacrylate, pentaerythritol divinyl ether, vinyl acrylate, divinyl benzene, divinyl succinate, diallyl phthalate, triallyl phosphate, triallyl isocyanurate or tris(2-acryloylethyl)isocyanurate. Examples of high molecular weight (oligomeric) polyunsaturated compounds are acrylated epoxy resins, acrylated polyethers, acrylated polyurethanes and acrylated polyesters. Further examples of unsaturated oligomers are unsaturated polyester resins, which are usually prepared from maleic acid, phthalic acid and one or more diols and which have molecular weights of greater than about 500. Unsaturated oligomers of this type are also known as prepolymers.

Typical examples of unsaturated compounds are esters of ethylenically unsaturated carboxylic acids and polyols or polyepoxides, and polymers containing ethylenically unsaturated groups in the chain or in side groups, including unsaturated polyesters, polyamides and polyurethanes and copolymers thereof, polybutadiene and butadiene copolymers, polyisoprene and isoprene copolymers, polymers and copolymers containing (meth)acrylic groups in side-chains, as well as mixtures of one or more than one such polymer.

Illustrative examples of unsaturated carboxylic acids are acrylic acid, methacrylic acid, crotonic acid, itaconic acid, cinnamic acid, unsaturated fatty acids such as linolenic acid or oleic acid. Acrylic and methacrylic acid are preferred.

Suitable polyols are aromatic and, preferably, aliphatic and cycloaliphatic polyols. Aromatic polyols are typically hydroquinone, 4,4′-dihydroxydiphenyl, 2,2-bis(4-hydroxyphenyl)propane, as well as novolacs and cresols. Polyepoxides include those based on the cited polyols, preferably on the aromatic polyols and epichlorohydrin. Further suitable polyols are polymers and copolymers which contain hydroxyl groups in the polymer chain or in side groups, for example polyvinyl alcohol and copolymers thereof or hydroxyalkyl polymethacrylates or copolymers thereof. Other suitable polyols are oligoesters carrying hydroxyl end groups.

Illustrative examples of aliphatic and cycloaliphatic polyols are alkylenediols containing preferably 2 to 12 carbon atoms, including ethylene glycol, 1,2- or 1,3-propanediol, 1,2-, 1,3- or 1,4-butanediol, pentanediol, hexanediol, octanediol, dodecanediol, diethylene glycol, triethylene glycol, polyethylene glycols having molecular weights of preferably 200 to 1500, 1,3-cyclopentanediol, 1,2-, 1,3-or 1,4-cyclohexanediol, 1,4-dihydroxymethylcyclohexane, glycerol, tris(□-hydroxyethyl)amine, trimethylolethane, trimethylolpropane, pentaerythritol, dipentaerythritol and sorbitol.

The polyols may be esterified partially or completely with one or with different unsaturated carboxylic acids, in which case the free hydroxyl groups of the partial esters may be modified, for example etherified, or esterified with other carboxylic acids.

Illustrative examples of esters are: Trimethylolpropane triacrylate, trimethylolethane triacrylate, trimethylolpropane trimethacrylate, trimethylolethane trimethacrylate, tetramethylene glycol dimethacrylate, triethylene glycol dimethacrylate, tetraethylene glycol diacrylate, pentaerythritol diacrylate, pentaerythritol triacrylate, pentaerythritol tetraacrylate, dipentaerythritol diacrylate, dipentaerythritol triacrylate, dipentaerythritol tetraacrylate, dipentaerythritol pentacrylate, dipentaerythritol hexacrylate, tripentaerythritol octacrylate, pentaerythritol dimethacrylate, pentaerythritol trimethacrylate, dipentaerythritol dimethacrylate, dipentaerythritol tetramethacrylate, tripentaerythritol octamethacrylate, pentaerythritol diitaconate, dipentaerythritol trisitaconate, dipentaerythritol pentaitaconate, dipentaerythritol hexaitaconate, ethylene glycol diacrylate, 1,3-butanediol diacrylate, 1,3-butanediol dimethacrylate, 1,4-butanediol diitaconate, sorbitol triacrylate, sorbitol tetraacrylate, pentaerythritol-modified triacrylate, sorbitol tetramethacrylate, sorbitol pentacrylate, sorbitol hexacrylate, oligoester acrylates and methacrylates, glycerol di- and-triacrylate, 1,4-cyclohexanediacrylate, bisacrylates and bismethacrylates of polyethylene glycol having molecular weights of 200 to 1500, or mixtures thereof. Polyfunctional monomers and oligomers are available for example from UCB Chemicals, Smyrna, Ga., and Sartomer, Exton, Pa.

Suitable ethylenically unsaturated polymerizable compounds are also the amides of identical or different unsaturated carboxylic acids of aromatic, cycloaliphatic and aliphatic polyamines containing preferably 2 to 6, more particularly 2 to 4, amino groups. Exemplary of such polyamines are ethylenediamine, 1,2- or 1,3-propylenediamine, 1,2-,1,3-or 1,4-butylenediamine, 1,5-pentylenediamine, 1,6-hexylenediamine, octylenediamine, dodecylenediamine, 1,4-diaminocyclohexane, isophoronediamine, phenylenediamine, bisphenylenediamine, bis(β-aminoethyl) ether, diethylenetriamine, triethylenetetramine, bis(β-aminoethoxy)ethane or bis(β-aminopropoxy)ethane. Other suitable polyamines are polymers and copolymers which may contain additional amino groups in the side-chain and oligoamides containing amino end groups.

Exemplary of such unsaturated amides are: Methylenebisacrylamide, 1,6-hexamethylenebisacrylamide, diethylenetriaminetrismethacrylamide, bis(methacrylamidopropoxy)ethane, β-methacrylamidoethylmethacrylate, N-[(β-hydroxyethoxy)ethyl]acrylamide.

Suitable unsaturated polyesters and polyamides are derived typically from maleic acid and diols or diamines. Maleic acid can be partially replaced by other dicarboxylic acids such as fumaric acid, itaconic acid, citraconic acid, mesaconic acid or chloromaleic acid. To control the reactivity of the polyester and to influence the crosslinking density and hence the product properties, it is possible to use in addition to the unsaturated dicarboxylic acids different amounts of saturated dicarboxylic acids such as phthalic acid, isophthalic acid, terephthalic acid, tetrahydrophthalic acid, succinic acid or adipic acid. The unsaturated polyesters can be used together with ethylenically unsaturated comonomers such as styrene. The polyesters and polyamides can also be derived from dicarboxylic acids and ethylenically unsaturated diols or diamines, especially from those with long chains containing typically from 6 to 20 carbon atoms. Polyurethanes are typically those derived from saturated or unsaturated diisocyanates and unsaturated and saturated diols.

Suitable polyester acrylates or acrylated polyesters are obtained by reacting oligomers, typically epoxides, urethanes, polyethers or polyesters, with acrylates such as hydroxyethyl acrylate or hydroxypropyl acrylate.

Polybutadiene and polyisoprene and copolymers thereof are known. Suitable comonomers include olefins such as ethylene, propene, butene, hexene, (meth)acrylates, acrylonitrile, styrene or vinyl chloride. Polymers containing (meth)acrylate groups in the side-chain are also known. They may typically be reaction products of epoxy resins based on novolak with (meth)acrylic acid, homo- or copolymers of polyvinyl alcohol or their hydroxyalkyl derivatives which are esterified with (meth)acrylic acid or homo- and copolymers of (meth)acrylates which are esterified with hydroxyalkyl(meth)acrylates.

Preferred monomers are typically alkyl- or hydroxyalkyl acrylates or methacrylates, styrene, ethylene glycol diacrylate, propylene glycol diacrylate, neopentyl glycol diacrylate, hexamethylene glycol diacrylate or bisphenol A diacrylate, 4,4′-bis(2-acryloyloxyethoxy)diphenylpropane, trimethylolpropane triacrylate, pentaerythritol triacrylate or tetraacrylate, preferably acrylates, styrene, hexamethylene glycol or bisphenol A diacrylate, 4,4′-bis(2-acryloyloxyethoxy)diphenylpropane or trimethylolpropane triacrylate.

Particularly preferred (oligomeric) polyunsaturated compounds are polyester acrylates or unsaturated polyester resins which are prepared from maleic acid, fumaric acid, phthalic acid and one or more than one diol, and which typically have molecular weights from about 500 to 3000.

Preferred unsaturated carboxylic acids are acrylic acid and methacrylic acid.

The photopolymerizable compounds are used by themselves or in any desired mixtures. It is preferred to use mixtures of polyol(meth)acrylates.

Binders may also be added to the unsaturated photopolymerizable compounds. The addition of binders is particularly useful if the photopolymerizable compounds are liquid or viscous substances. The amount of binder may be from 5-95, preferably 10-90 and, most preferably, 40-90, percent by weight, based on the entire composition. The choice of binder will depend on the field of use and the desired properties therefore, such as the ability of the compositions to be developed in aqueous and organic solvent systems, adhesion to substrates and susceptibility to oxygen.

Suitable binders are typically polymers having a molecular weight of about 5,000 to 2,000,000, preferably 10,000 to 1,000,000. Illustrative examples are: Homo- and copolymers of acrylates and methacrylates, including copolymers of methyl methacrylate/ethyl acrylate/methacrylic acid, poly(alkylmethacrylates), poly(alkylacrylates); cellulose esters and ethers such as cellulose acetate, cellulose acetobutyrate, methyl cellulose, ethyl cellulose; polyvinyl butyral, polyvinyl formal, cyclized rubber, polyethers such as polyethylene oxide, polypropylene oxide, polytetrahydrofuran; polystyrene, polycarbonate, polyurethane, chlorinated polyolefins, polyvinyl chloride, copolymers of vinyl chloride/vinylidene chloride, copolymers of vinylidene chloride with acrylonitrile, methyl methacrylate and vinyl acetate, polyvinyl acetate, copoly(ethylene/vinyl acetate), polymers such as polycaprolactam and poly(hexamethylene adipamide), polyesters such as poly(ethylene glycol terephthalate) and poly(hexamethylene glycol succinate).

The unsaturated compounds can also be used in admixture with non-photopolymerizable film-forming components. These components may be physically drying polymers or solutions thereof in organic solvents, for example nitrocellulose or cellulose acetobutyrate. The photopolymerizable unsaturated monomers may be a component of a free radical-ionic curable blend, such as a free radical-cationic curable blend. Also of importance are systems that undergo both thermal and photo-induced curing cycles, such as are used in powder coatings, laminates, certain adhesives and conformal coatings.

Mixtures of a prepolymer with polyunsaturated monomers which, additionally contain a further unsaturated monomer are frequently used in paint systems. The prepolymer in this instance primarily determines the properties of the paint film and, by varying it, the skilled person can influence the properties of the cured film. The polyunsaturated monomer acts as crosslinking agent that renders the paint film insoluble. The mono-unsaturated monomer acts as reactive diluent with the aid of which the viscosity is lowered without having to use a solvent. Moreover, properties of the cured composition such as curing rate, crosslinking density and surface properties are dependent on the choice of monomer.

Unsaturated polyester resins are usually used in two-component systems, together with a mono-unsaturated monomer, preferably with styrene.

Binary electron-rich/electron-poor monomer systems are often employed in thick pigmented coatings. For example, vinyl ether/unsaturated polyester systems are employed in powder coatings and styrene/unsaturated polyester systems are used in gel coats.

A preferred process is that wherein the ethylenically unsaturated polymerizable compounds are a mixture of (i) at least one oligomeric compound and (ii) at least one monomer.

An interesting process is that wherein the ethylenically unsaturated polymerizable compounds are a mixture of (i) unsaturated polyesters, especially those that are prepared from maleic acid, fumaric acid and/or phthalic acid and one or more than one diol, and which have molecular weights of 500 to 3,000, and (ii) acrylates, methacrylates or styrene or combinations thereof.

An important process is also that wherein the ethylenically unsaturated polymerizable compounds are a mixture of (i) unsaturated polyesters and (ii) acrylates or methacrylates or combinations thereof.

Another interesting process is that wherein the ethylenically unsaturated polymerizable compounds are a mixture of (i) unsaturated polyester acrylates and (ii) acrylates or methacrylates or combinations thereof.

The amount of instant stabilizer compounds employed in the compositions of this invention are for example from about 0.001% to about 10% by weight, based on the weight of the composition and depending on the composition.

For example, the amount of the present stabilizers employed in body care products, household products, textiles and fabrics is from about 0.001% to about 10% by weight, or from about 0.001% to about 5% by weight, based on the weight of the composition.

The amount of instant stabilizer compounds employed in coatings is from about 0.1 to about 10% by weight, for example from about 0.2 to about 5% by weight, for example from about 0.5 to about 3% by weight based on the weight of the solvent-free binder. The binders can be dissolved or dispersed in customary organic solvents or in water or can be solvent-free.

If combinations of stabilizers are used, the sum of all light stabilizers is for example from about 0.2 to about 20% by weight, for instance from about 0.5 to about 5% by weight, based on the film-forming resin.

It is also contemplated that the instant compounds would find particular value when used with water soluble inks and related polar oriented utilities where the presence of the water solubilizing groups would provide for better compatibility and properties related to such aqueous environments.

Other materials that are stabilized according to the instant invention include recording materials such as photographic reproductions or reprographic materials. The novel recording materials also include, for example, pressure-sensitive copying systems, microcapsule photocopier systems, heat-sensitive copier systems, photographic material and ink-jet printing. The various components of ink jet media are disclosed for example in U.S. Pat. Nos. 4,503,111, 4,575,465, 4,935,307, 5,206,071, 6,096,826 and 6,127,037 and U.S. app. No. 60/406,441, filed Aug. 28, 2002, the relevant parts of which are hereby incorporated by reference.

The ink jet ink according to this invention comprises about 0.01 to about 30% by weight, for example about 0.1 to about 20% by weight, of at least one present sterically hindered amine stabilizer, based on the weight of the ink jet ink.

The ink jet recording material according to this invention comprises about 1 to about 10000 mg/m², for example about 50 to about 2000 mg/m², of at least one present sterically hindered amine stabilizer.

The present sterically hindered amine stabilizers are preferably added to casting or coating dispersions which are applied by customary techniques to the support of the ink jet recording material, or they can be absorbed onto the material from an aqueous or organic solution. If the recording material contains more than one layer, the compounds according to this invention can be added to one layer or can be distributed over a plurality of layers, wherein they can be applied to a plurality of layers in the same or different concentrations.

The present sterically hindered amine stabilizers are preferably used in ink jet inks or recording materials, but may also be incorporated in ink compositions for felt-tipped pens, ink pads, fountain pens, and pen plotters, as well as for offset, book, flexographic and intaglio printing, and also for typewriter ribbons for dot matrix and calligraphic printing. The sterically hindered amine stabilizers can further be used in silver halide photographic materials as well as in recording materials for pressure-sensitive copying systems, microcapsule photocopier systems, heat-sensitive copier systems, dye diffusion transfer printing, thermal wax transfer printing and dot matrix printing, and for use with electrostatic, electrographic, electrophoretic, magnetographic and laser-electrophotographic printers, recorders or plotters.

Amongst the printers used for ink jet printing, a distinction is usually made between continuous and drop-on-demand printers. The ink jet system according to this invention is suited for use with both type of printers.

The ink compositions according to the novel ink jet system are preferably water borne inks and may contain water soluble solvents such as ethylene glycol, diethylene glycol, triethylene glycol or higher ethylene glycols, propylene glycol, 1,4-butanediol, or ethers of such glycols, thiodiglycol, glycerol and the ethers and esters thereof, polyglycerol, mono-, di- and triethanolamine, propanolamine, dimethyl formamide, dimethyl sulfoxide, dimethyl acetamide, N-methylpyrrolidone, 1,3-dimethylimidazolidone, methanol, ethanol, isopropanol, n-propanol, diacetone alcohol, acetone, methyl ethyl ketone or propylene carbonate.

The ink compositions according to the novel ink jet system preferably contain water soluble dyes, such as those known for dyeing natural fibres. These can, for example, be acid dyes, direct dyes, reactive dyes, mono-, di- or polyazo dyes, triphenylmethane dyes, xanthene dyes or phtalocyanine dyes. Specific examples of such dyes are Food Black 2, Direct Black 19, Direct Black 38, Direct Black 168, Sulphur Black 1, Acid Red 14, Acid Red 35, Acid Red 52, Acid Red 249, Direct Red 227, Reactive Red 24, Reactive Red 40, Reactive Red 120, Reactive Red 159, Reactive Red 180, Acid Yellow 17, Acid Yellow 23, Direct Yellow 86, Direct Yellow 132, Acid blue 9, Acid Blue 185, Direct Blue 86, Direct Blue 199, copper phtalocyanines and the azo dyes listed in EP-A-366 221.

The ink compositions according to the invention may be nonaqueous and consist of a solution of dyes in an organic solvent or a mixture of organic solvents. Examples of solvents used for this purpose are alkyl carbitols, alkylcellosolves, dialkylformamides, dialkylacetamides, alcohols, acetone, methylethylketone, diethylketone, methyl isobutyl ketone, diisopropyl ketone, dibutyl ketone, dioxane, ethyl butyrate, ethyl isovalerate, diethyl malonate, diethyl succinate, butyl acetate, triethyl phosphate, ethylglycol acetate, toluene, xylene, tetralin or petroleum fractions. Example of solid waxes as solvents, which, as an ink carrier, must first be heated, are stearic or palmiric acid. Solvent based inks contain dyes soluble therein, for example Solvent Red, Solvent Yellow, Solvent Orange, Solvent Blue, Solvent Green, Solvent Violet, Solvent Brown or Solvent Black.

The ink compositions according to the novel ink jet system may also contain minor amounts of conventional modifiers such as binders, surfactants, biocides, corrosion inhibitors, sequestrants, pH buffers or conductivity additives. They may also contain further light stabilizers or UV absorbers, including the compounds disclosed in U.S. Pat. Nos. 5,073,448, 5,089,050, 5,096,489, 5,124,723, 5,098,477 and 5,509,957.

The ink compositions according to the invention may also consist of more than one phase. Ink compositions that consist of an aqueous phase in which the dye is dissolved and a dispersed oil phase that contains an UV absorber and/or an antioxidant are for example disclosed in JP-A-01170 675, JP-A-01182 379, JP-A-01182 380, JP-A-01182 381, JP-A-01193 376. Oil soluble dyes can be dissolved in an oil together with UV absorbers and/or antioxidants. The oil is either emulsified or dispersed in an aqueous phase as described, inter alia, in JP-A-01170674 and JP-A-01170672.

Further suited ink jet ink compositions are described in EP-A-672 538, pages 3 to 6.

The recording materials according to the novel ink jet system consist of a substrate having a surface which is printable by means of an ink jet. The substrate is usually plain paper or polyolefin-laminated paper or a plastic sheet and is usually coated with at least one layer which is able to absorb ink. The substrate preferably has a thickness of 80 to 250 μm.

Uncoated paper might also be used. In this case, the paper acts simultaneously as substrate and ink absorbing layer. Materials made of cellulosic fibers and textile fibers materials such as cotton fabrics or blends of cotton and polyacrylamide or polyester, which might contain the present sterically hindered amine light stabilizers, can also be used as printing materials.

The recording materials may also be transparent, as in the case of overhead projection transparencies.

The present sterically hindered amine stabilizers can be incorporated in the substrate during production thereof, conveniently by addition to the pulp during paper manufacture. Another method of application consists in spraying the substrate with a solution of the present sterically hindered amine stabilizers in water or in a readily volatile organic solvent. The use of emulsions or dispersions is also possible.

Usually, however, at least one coating composition with high dye affinity is coated onto the substrate and, in this case, the present sterically hindered amines are added to at least one of the said coating compositions. Typical coating compositions comprise, for example, a solid filler, a binder and conventional additives.

Example of suitable fillers are SiO₂, kaolin, talc, clay, calcium silicate, magnesium silicate, aluminium silicate, gypsum, zeolites, bentonite, diatomaceous earth, vermiculite, starch or the surface modified SiO₂ described in JP-A-60 260 377. Small amounts of white pigments, for example titanium dioxide, barytes, magnesium oxide, lime, chalk or magnesium carbonate, can be used with the filler in the coating composition, provided they do not significantly lower the print density of the ink jet prints.

The present sterically hindered amines may advantageously be employed in a nanoporous or microporous ink jet material.

Coating compositions which are intended for transparent, projectable recording materials must not contain any light-scattering particles, such as pigments and fillers.

The binder binds the fillers to one another and to the substrate. Typical conventional binders are water soluble polymers such as polyvinyl alcohol, partially hydrolysed polyvinyl acetate, cellulose and cellulose derivatives such as hydroxyethyl cellulose, polyvinyl pyrrolidone and copolymers thereof, polyethylene oxide, salts of polyacrylic acid, sodium alginate, starch and starch derivatives, Na alginate, polyethylene imine, polyvinylpyridinium halide, gelatines and gelatine derivatives such as phthaloyl gelatines, casein, vegetable gum, dextrin, albumin, dispersions and polyacrylates or acrylate/methacrylate copolymers, lattices of natural or synthetic rubber, poly(meth)acrylamide, polyvinyl ethers, polyvinyl esters, copolymers of maleic acid, melamine resins, urea resins, water soluble polyurethanes and polyesters, or the chemically modified polyvinyl alcohols disclosed in JP-A-61 134 290 or JP-A-61 134 291.

An additional dye receptor or a mordant which enhances the fixation of the dye to the coating may be added to the binder. Dye receptors for acid dyes are cationic or amphoteric. The cationic mordants can be soluble or dispersible in water. Exemplary cationic mordants are polymeric ammonium compounds such as polyvinylbenzyldi- or trialkylammonium compounds, optionally quaternized poly(di)allylammonium compounds, polymethacryloxyethyldimethylhydroxyethylammonium chloride, polyvinylbenzylmethylimidazolium chloride, polyvinylbenzylpicolinium chloride or polyvinylbenzyltributylammonium chloride. Further examples are basic polymers such as poly(dimethylaminoethyl)methacrylate, polyalkylenepolyamines and their condensation products with dicyanodiamide, amine/epichlorohydrin polycondensates or the compounds disclosed in JP-A-57-36692, 57-64591, 57-187289, 57-191084, 58-177390, 58-208357, 59-20696, 59-33176, 59-96987, 59-198188, 60-49990, 60-71796, 60-72785, 60-161188, 60-187 582, 60-189481, 60-189482, 61-14979, 61-43593, 61-57379, 61-57380, 61-58788, 61-61887, 61-63477, 61-72581, 61-95977, 61-134291 or in U.S. Pat. Nos. 4,547,405 and 4,554,181 as well as in DE-A-3417582 and EP-B-609 930. The mordants used can also be compounds containing phosphonium groups (EP-B-609 930) as well as ground cationic ion exchange resins which are introduced in the mordant layer in a finely divided form. Further suitable cationic mordants are described in U.S. Pat. No. 6,102,997, pages 12 to 17. The cationic mordants can be soluble or dispersible in water and have an average molecular weight (weight average) of preferably at least 2,000 and, in particular, at least 20,000.

Besides the dye acceptor layer(s), the ink jet recording material might comprise other layers on the ink receiving side, which are intended, for example, for providing scratch resistance, absorbing water or controlling whiteness and/or glossiness. The backside of the substrate might also be coated with at least one binder layer, in order to prevent buckling of the recording material.

The ink jet recording material might also contain a number of other additives such as antioxidants, further light stabilizers (also including UV absorbers), viscosity improvers, fluorescent whitening agents, biocides, wetting agents, emulsifiers and spacers.

Suitable spacers are in particular spherical, have an average diameter of 1 to 50 μm, and in particular 5 to 20 μm, and have a narrow particle size distribution. Suitable spacers consist, for example, of polymethylmethacrylate, polystyrene, polyvinyl toluene, silicon dioxide and insoluble starch.

Illustrative examples of particularly suitable antioxidants are sterically hindered phenols, hydroquinones and hydroquinone ethers, for example the antioxidants disclosed in GB-A-2 088 777 or JP-A-60-72785, JP-A-0-72786 and JP-A-60-71796.

Illustrative examples of particularly suitable light stabilizers are organic nickel compounds and sterically hindered amines, for example the light stabilizers disclosed in JP-A-58-152072, 61-146591, 61-163886, 60-72785 and 61-146591 or in EP 373 573, 685 345 and 704 316, GB-A-2 088 777, JP-A-59-169883 and 61-177279.

Suitable UV absorbers are disclosed, inter alia, in Research Disclosure No. 24239 (1984) page 284, 37254 part VIII (1995) page 292, 37038 part X (1995) page 85 and 38957 part VI (1996), GB-A-2 088 777, EP 280 650, EP 306 083 and EP 711 804. These compounds are preferably introduced into the layer(s) farthest from the support. In a particular embodiment, the UV absorbers are contained in a layer above the layer(s) containing the present sterically hindered amines. Suitable UV absorbers for concurrent use with a present sterically hindered amines in recording materials for ink jet printing are in particular those of the 2′-hydroxyphenylbenzotriazole and 2′-hydroxyphenyltriazine class and, most particularly, 2-(2′-hydroxy-3′,5′-di-tert-amylphenyl)benzotriazole and 2-(2′-hydroxy-3′-tert-butyl-5′-polyglycolpropionate-phenyl)benzotriazole. Further examples of particularly suited UV absorbers are listed in U.S. Pat. No. 6,102,997 pages 18-19. The UV absorbers can be soluble or insoluble in water and added to the coating composition as dispersion or emulsion, optionally together with high-boiling solvents, using suitable dispersing agents or emulsifiers. Suitable high boiling solvents are described in Research Disclosure No. 37254 part VIII (1995) page 292.

The binders in the individual layers, and in particular gelatines, can also be crosslinked by suitable compounds, so-called hardening agents, in order to improve the water and scratch resistance of the layers. Suitable hardening agents are described in Research Disclosure No. 37254 part IX (1995) page 294, 37038 part XII (1995) page 86 and 38957 part IIB (1996) page 599 et seq. The hardening agents are normally used in quantities of 0.005 to 10% by weight, and preferably 0.01 to 1% by weight, based on the binder to be hardened.

The ink jet recording material can be produced in one pass from the support material and a casting solution for each layer to be applied, by means of a cascade or curtain casting device of the kind known from the production of photographic silver halide materials. After the casting solution(s) has/have been cast on the support, the material is dried and is then ready for use. The individual layers have a dry layer thickness of 0.1 to 20 μm, and preferably 0.5 to 5 μm.

The present sterically hindered amines can be dissolved either directly in the ink or coating composition or added thereto in the form of an emulsion or suspension. As already mentioned, the present sterically hindered amines can be also applied to the recording material in a separate operation, alone or together with other already described components, as a solution in water or in a suitable organic solvent. Application can be made by spraying, by sizing in a sizing press, by a separate coating operation or by immersion in a vat. After subjecting the recording material to such an after treatment, an additional drying step is necessary.

The present photographic material can be a black and white or a color photographic material, color photographic material is preferred. Further details on the structure of color photographic material and the components which can be employed in such materials are described in U.S. Pat. No. 5,538,840 at column 27, line 25 to column 106, line 16. These relevant parts are incorporated herein by reference. Application of the instant novel compounds is essentially as described for UV absorbers or hindered amine stabilizers in U.S. Pat. No. 5,538,840.

Further important components, especially couplers, are described in U.S. Pat. No. 5,578,437.

Other articles which would benefit from the incorporation of the instant, water compatible hindered amine compounds include laminated articles as described in U.S. Pat. Nos. 6,268,415 and 6,191,199, the disclosures of which are hereby incorporated by reference. For example, laminated articles such as:

(a) Retroreflective Sheets and Signs and Conformable Marketing Sheets as seen in WO 97/42261; and U.S. Pat. No. 5,387,458 which is incorporated herein by reference;

(b) Solar Control Films of Various Construction as seen in British 2,012,668; European 355,962; and U.S. Pat. Nos. 3,290,203; 3,681,179; 3,776,805 and 4,095,013 which are incorporated herein by reference;

(c) Corrosion Resistant Silver Mirrors and Solar Reflectors as seen in U.S. Pat. No. 4,645,714 which is incorporated herein by reference;

(d) Reflective Print Labels as seen in U.S. Pat. No. 5,564,843 which is incorporated herein by reference;

(e) UV Absorbing Glasses and Glass Coatings as seen in U.S. Pat. Nos. 5,372,889; 5,426,204; 5,683,804 and 5,618,626 which are incorporated herein by reference;

(f) Electrochromic Devices as seen in European 752,612 A1; and U.S. Pat. Nos. 5,239,406; 5,523,877 and 5,770,114 which are incorporated herein by reference;

(g) Films/Glazings as seen in WO 92/01557; Japanese Nos. 75-33286; 93-143668; 95-3217 and 96-143831; and U.S. Pat. No. 5,643,676 which is incorporated herein by reference;

(h) Windscreens and Intermediate Layers as seen in Japanese Nos. 80-40018; 90-192118; 90-335037; 90-335038; 92-110128 and 94-127591; and

(i) Optical Films as seen in WO 97/32225; and U.S. Pat. Nos. 4,871,784 and 5,217,794 which are incorporated herein by reference.

As mentioned, the stabilized compositions of the invention may optionally also contain other conventional stabilizers. For example, the compositions of this invention may contain from about 0.01 to about 5%, preferably from about 0.025 to about 2%, and especially from about 0.1 to about 1% by weight of various conventional additives, such as the materials listed below, or mixtures thereof.

1. Antioxidants

1.1. Alkylated monophenols, for example 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1-methylundec-1-yl)phenol, 2,4-dimethyl-6-(1-methylheptadec-1-yl)phenol, 2,4-dimethyl-6-(1-methyltridec-1-yl)phenol and mixtures thereof.

1.2. Alkylthiomethylphenols, for example 2,4-dioctylthiomethyl-6-tert-butylphenol, 2,4-dioctylthiomethyl-6-methylphenol, 2,4-dioctylthiomethyl-6-ethylphenol, 2,6-di-dodecylthiomethyl-4-nonylphenol.

1.3. Hydroquinones and alkylated hydroquinones, for example 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis-(3,5-di-tert-butyl-4-hydroxyphenyl) adipate.

1.4. Tocopherols, for example α-tocopherol, β-tocopherol, γ-tocopherol, δ-tocopherol and mixtures thereof (Vitamin E).

1.5. Hydroxylated thiodiphenyl ethers, for example 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis-(3,6-di-sec-amylphenol), 4,4′-bis(2,6-dimethyl-4-hydroxyphenyl)disulfide.

1.6. Alkylidenebisphenols, for example 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3-tert-butyl-4-hydroxyphenyl)butyrate], bis(3-tert-butyl-4-hydroxy-5-methyl-phenyl)dicyclopentadiene, bis[2-(3′tert-butyl-2-hydroxy-5-methylbenzyl)-6-tert-butyl-4-methylphenyl]terephthalate, 1,1-bis-(3,5-dimethyl-2-hydroxyphenyl)butane, 2,2-bis-(3,5-di-tert-butyl-4-hydroxyphenyl)propane, 2,2-bis-(5-tert-butyl-4-hydroxy2-methylphenyl)-4-n-dodecylmercaptobutane, 1,1,5,5-tetra-(5-tert-butyl-4-hydroxy-2-methylphenyl)pentane.

1.7. Benzyl compounds, for example 3,5,3′,5′-tetra-tert-butyl-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, 1,3,5-tri-(3,5-di-tert-butyl-4-hydroxybenzyl)-2,4,6-trimethylbenzene, di-(3,5-di-tert-butyl-4-hydroxybenzyl) sulfide, 3,5-di-tert-butyl-4-hydroxybenzyl-mercapto-acetic acid isooctyl ester, bis-(4-tert-butyl-3-hydroxy-2,6-dimethylbenzyl)dithiol terephthalate, 1,3,5-tris-(3,5-di-tert-butyl-4-hydroxybenzyl) isocyanurate, 1,3,5-tris-(4-tert-butyl-3-hydroxy-2,6-dimethylbenzyl) isocyanurate, 3,5-di-tert-butyl-4-hydroxybenzyl-phosphoric acid dioctadecyl ester and 3,5-di-tert-butyl-4-hydroxybenzyl-phosphoric acid monoethyl ester, calcium-salt.

1.8. Hydroxybenzylated malonates, for example dioctadecyl-2,2-bis-(3,5-di-tert-butyl-2-hydroxybenzyl)-malonate, di-octadecyl-2-(3-tert-butyl-4-hydroxy-5-methylbenzyl)-malonate, di-dodecylmercaptoethyl-2,2-bis-(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate.

1.9. Aromatic hydroxybenzyl compounds, for example 1,3,5-tris-(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol.

1.10. Triazine compounds, for example 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris-(3,5-di-tert-butyl-4-hydroxyphenoxy)-1,2,3-triazine, 1,3,5-tris-(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris(3,5-di-tert-butyl-4-hydroxyphenylpropionyl)-hexahydro-1,3,5-triazine, 1,3,5-tris(3,5-dicyclohexyl-4-hydroxybenzyl)isocyanurate.

1.11. Benzylphosphonates, for example dimethyl-2,5-di-tert-butyl-4-hydroxybenzylphosphonate, diethyl-3,5-di-tert-butyl-4-hydroxybenzylphosphonate, dioctadecyl3,5-di-tert-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid.

1.12. Acylaminophenols, for example 4-hydroxy-lauric acid anilide, 4-hydroxy-stearic acid anilide, 2,4-bis-octylmercapto-6-(3,5-tert-butyl-4-hydroxyanilino)-s-triazine and octyl-N-(3,5-di-tert-butyl-4-hydroxyphenyl)-carbamate.

1.13. Esters of β-(3,5-di-tert-butyl-4-hydroxyphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl) isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.14. Esters of β-(5-tert-butyl-4-hydroxy-3-methylphenyl)prorionic acid with mono- or polyhydric alcohols, e.g. with methanol, ethanol, n-octanol, i-octanol, octadecanol, 1,6-hexane-diol, 1,9-nonanediol, ethylene glycol, 1,2-propanediol, neopentyl glycol, thiodiethylene glycol, diethylene glycol, triethylene glycol, pentaerythritol, tris(hydroxyethyl) isocyanurate, N,N′-bis-(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.15. Esters of β-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.16. Esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.17. Amides of β-(3,5-di-tert-butyl-4-hydroxyphenyl)propionic acid e.g. N,N′-bis(3,5-di-tert-butyl-4-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard®XL-1 supplied by Uniroyal).

1.18. Ascorbic acid (vitamin C)

1.19. Aminic antioxidants, for example N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′phenyl-p-phenylenediamine, N-cyclohexyl-N′-phenyl-p-phenlenediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, for example p,p′-di-tert-octyidiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine, 2,6-di-tert-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyl-diphenylamines, a mixture of mono- and dialkylated nonyldiphenylamines, a mixture of mono- and dialkylated dodecyidiphenylamines, a mixture of mono- and dialkylated isopropyl/isohexyldiphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octyl-phenothiazines, N-allylphenothiazin, N,N,N′,N′-tetraphenyl-1,4-diaminobut-2-ene, N,N-bis-(2,2,6,6-tetramethyl-piperid-4-yl-hexamethylenediamine, bis(2,2,6,6-tetramethylpiperid-4-yl)-sebacate, 2,2,6,6-tetramethylpiperidin-4-one, 2,2,6,6-tetramethylpiperidin-4-ol.

2. UV absorbers and light stabilizers

2.1. 2-(2-Hydroxyphenyl)-2H-benzotriazoles, for example known commercial hydroxyphenyl-2H-benzotriazoles and benzotriazoles as disclosed in, U.S. Pat. Nos. 3,004,896; 3,055,896; 3,072,585; 3,074,910; 3,189,615; 3,218,332; 3,230,194; 4,127,586; 4,226,763; 4,275,004; 4,278,589; 4,315,848; 4,347,180; 4,383,863; 4,675,352; 4,681,905, 4,853,471; 5,268,450; 5,278,314; 5,280,124; 5,319,091; 5,410,071; 5,436,349; 5,516,914; 5,554,760; 5,563,242; 5,574,166; 5,607,987, 5,977,219 and 6,166,218 such as 2-(2-hydroxy-5-methylphenyl)-2H-benzotriazole, 2-(3,5-di-t-butyl-2-hydroxyphenyl)-2H-benzotriazole, 2-(2-hydroxy-5-t-butylphenyl)-2H-benzotriazole, 2-(2-hydroxy-5-t-octylphenyl)-2H-benzotriazole, 5-chloro-2-(3,5-di-t-butyl-2-hydroxyphenyl)-2H-benzotriazole, 5-chloro-2-(3-t-butyl-2-hydroxy-5-methylphenyl)-2H-benzotriazole, 2-(3-sec-butyl-5-t-butyl-2-hydroxyphenyl)-2H-benzotriazole, 2-(2-hydroxy-4-octyloxyphenyl)-2H-benzotriazole, 2-(3,5-di-t-amyl-2-hydroxyphenyl)-2H-benzotriazole, 2-(3,5-bis-α-cumyl-2-hydroxyphenyl)-2H-benzotriazole, 2-(3-t-butyl-2-hydroxy-5-(2-(ω-hydroxy-octa-(ethyleneoxy)carbonyl-ethyl)-, phenyl)-2H-benzotriazole, 2-(3-dodecyl-2-hydroxy-5-methylphenyl)-2H-benzotriazole, 2-(3-t-butyl-2-hydroxy-5-(2-octyloxycarbonyl)ethylphenyl)-2H-benzotriazole, dodecylated 2-(2-hydroxy-5-methylphenyl)-2H-benzotriazole, 2-(3-t-butyl-2-hydroxy-5-(2-octyloxycarbonylethyl)phenyl)-5-chloro-2H-benzotriazole, 2-(3-tert-butyl-5-(2-(2-ethylhexyloxy)-carbonylethyl)-2-hydroxyphenyl)-5-chloro-2H-benzotriazole, 2-(3-t-butyl-2-hydroxy-5-(2-methoxycarbonylethyl)phenyl)-5-chloro-2H-benzotriazole, 2-(3-t-butyl-2-hydroxy-5-(2-methoxycarbonylethyl)phenyl)-2H-benzotriazole, 2-(3-t-butyl-5-(2-(2-ethylhexyloxy)carbonylethyl)-2-hydroxyphenyl)-2H-benzotriazole, 2-(3-t-butyl-2-hydroxy-5-(2-isooctyloxycarbonylethyl)phenyl-2H-benzotriazole, 2,2′-methylene-bis(4-t-octyl-(6-2H-benzotriazol-2-yl)phenol), 2-(2-hydroxy-3-α-cumyl-5-t-octylphenyl)-2H-benzotriazole, 2-(2-hydroxy-3-t-octyl-5-α-cumylphenyl)-2H-benzotriazole, 5-fluoro-2-(2-hydroxy-3,5-di-α-cumylphenyl)-2H-benzotriazole, 5-chloro-2-(2-hydroxy-3,5-di-α-cumylphenyl)-2H-benzotriazole, 5-chloro-2-(2-hydroxy-3-α-cumyl-5-t-octylphenyl)-2H-benzotriazole, 2-(3-t-butyl-2-hydroxy-5-(2-isooctyloxycarbonylethyl)phenyl)-5-chloro-2H-benzotriazole, 5-trifluoromethyl-2-(2-hydroxy-3-α-cumyl-5-t-octylphenyl)-2H-benzotriazole, 5-trifluoromethyl-2-(2-hydroxy-5-t-octylphenyl)-2H-benzotriazole, 5-trifluoromethyl-2-(2-hydroxy-3,5-di-t-octylphenyl)-2H-benzotriazole, methyl 3-(5-trifluoromethyl-2H-benzotriazol-2-yl)-5-t-butyl-4-hydroxyhydrocinnamate, 5-butylsulfonyl-2-(2-hydroxy-3-α-cumyl-5-t-octylphenyl)-2H-benzotriazole, 5-trifluoromethyl-2-(2-hydroxy-3-α-cumyl-5-t-butylphenyl)-2H-benzotriazole, 5-trifluoromethyl-2-(2-hydroxy-3,5-di-t-butylphenyl)-2H-benzotriazole, 5-trifluoromethyl-2-(2-hydroxy-3,5-di-α-cumylphenyl)-2H-benzotriazole, 5-butylsulfonyl-2-(2-hydroxy-3,5-di-t-butylphenyl)-2H-benzotriazole and 5-phenylsulfonyl-2-(2-hydroxy-3,5-di-t-butylphenyl)-2H-benzotriazole.

2.2. 2-Hydroxybenzophenones, for example the 4-hydroxy, 4-methoxy, 4-octyloxy, 4-decyloxy, 4-dodecyloxy, 4-benzyloxy, 4,2′,4′-trihydroxy and 2′-hydroxy-4,4′-dimethoxy derivatives.

2.3. Esters of substituted and unsubstituted benzoic acids, as for example 4-tert-butylphenyl salicylate, phenyl salicylate, octylphenyl salicylate, dibenzoyl resorcinol, bis(4-tert-butylbenzoyl) resorcinol, benzoyl resorcinol, 2,4-di-tert-butylphenyl 3,5-di-tert-butyl-4-hydroxybenzoate, hexadecyl 3,5-di-tert-butyl-4-hydroxybenzoate, octadecyl 3,5-di-tert-butyl-4-hydroxybenzoate, 2-methyl-4,6-di-tert-butylphenyl 3,5-di-tert-butyl-4-hydroxybenzoate.

2.4. Acrylates and malonates, for example, α-cyano-β,β-diphenylacrylic acid ethyl ester or isooctyl ester, α-carbomethoxy-cinnamic acid methyl ester, α-cyano-β-methyl-p-methoxy-cinnamic acid methyl ester or butyl ester, α-carbomethoxy-p-methoxy-cinnamic acid methyl ester, N-(β-carbomethoxy-β-cyanovinyl)-2-methyl-indoline, Sanduvor® PR25, dimethyl p-methoxybenzylidenemalonate (CAS# 7443-25-6), and Sanduvor® PR31, di-(1,2,2,6,6-pentamethylpiperidin-4-yl) p-methoxybenzylidenemalonate (CAS #147783-69-5).

2.5. Nickel compounds, for example nickel complexes of 2,2′-thio-bis-[4-(1,1,3,3-tetramethylbutyl)phenol], such as the 1:1 or 1:2 complex, with or without additional ligands such as n-butylamine, triethanolamine or N-cyclohexyldiethanolamine, nickel dibutyldithiocarbamate, nickel salts of the monoalkyl esters, e.g. the methyl or ethyl ester, of 4-hydroxy-3,5-di-tert-butylbenzylphosphonic acid, nickel complexes of ketoximes, e.g. of 2-hydroxy-4-methylphenyl undecylketoxime, nickel complexes of 1-phenyl-4-lauroyl-5-hydroxypyrazole, with or without additional ligands.

2.6. Sterically hindered amine stabilizers, for example 4-hydroxy-2,2,6,6-tetramethylpiperidine, 1-allyl-4-hydroxy-2,2,6,6-tetramethylpiperidine, 1-benzyl-4-hydroxy-2,2,6,6-tetramethylpiperidine, bis(2,2,6,6-tetramethyl-4-piperidyl) sebacate, bis(2,2,6,6-tetramethyl-4-piperidyl) succinate, bis(1,2,2,6,6-pentamethyl-4-piperidyl) sebacate, bis(1-octyloxy-2,2,6,6-tetramethyl-4-piperidyl) sebacate, bis(1,2,2,6,6-pentamethyl-4-piperidyl) n-butyl-3,5-di-tert-butyl-4-hydroxybenzylmalonate, the condensate of 1-(2-hydroxyethyl)-2,2,6,6-tetramethyl-4-hydroxypiperidine and succinic acid, linear or cyclic condensates of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl) nitrilotriacetate, tetrakis(2,2,6,6-tetramethyl-4-piperidyl)-1,2,3,4-butane-tetracarboxylate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidyl)-2-n-butyl-2-(2-hydroxy-3,5-di-tert-butylbenzyl) malonate, 3-n-octyl-7,7,9,9-tetramethyl-1,3,8-triazaspiro[4.5]decan-2,4-dione, bis(1-octyloxy-2,2,6,6-tetramethylpiperidyl) sebacate, bis(1-octyloxy-2,2,6,6-tetramethylpiperidyl) succinate, linear or cyclic condensates of N,N′-bis-(2,2,6,6-tetramethyl-4-piperidyl)-hexamethylenediamine and 4-morpholino-2,6-dichloro-1,3,5-triazine, the condensate of 2-chloro-4,6-bis(4-n-butylamino-2,2,6,6-tetramethylpiperidyl )-1,3,5-triazine and 1,2-bis(3-aminopropylamino)ethane, the condensate of 2-chloro-4,6-di-(4-n-butylamino-1,2,2,6,6-pentamethylpiperidyl)-1,3,5-triazine and 1,2-bis-(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethyl-4-piperidyl)pyrrolidin-2,5-dione, 3-dodecyl-1-(1,2,2,6,6-pentamethyl-4-piperidyl)pyrrolidine-2,5-dione, a mixture of 4-hexadecyloxy- and 4-stearyloxy-2,2,6,6-tetramethylpiperidine, a condensation product of N,N′-bis(2,2,6,6-tetramethyl-4-piperidyl)hexamethylenediamine and 4-cyclohexylamino-2,6-dichloro-1,3,5-triazine, a condensation product of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]); N-(2,2,6,6-tetramethyl-4-piperidyl)-n-dodecylsuccinimid, N-(1,2,2,6,6-pentamethyl-4-piperidyl)-n-dodecylsuccinimid, 2-undecyl-7,7,9,9-tetramethyl-1-oxa-3,8-diaza-4-oxo-spiro[4,5]decane, a reaction product of 7,7,9,9-tetramethyl-2-cycloundecyl-1-oxa-3,8-diaza-4-oxospiro [4,5]decane and epichlorohydrin, 1,1-bis(1,2,2,6,6-pentamethyl-4-piperidyloxycarbonyl)-2-(4-methoxyphenyl)ethene, N,N′-bis-formyl-N,N′-bis(2,2,6,6-tetramethyl-4-piperidyl)hexamethylenediamine, diester of 4-methoxy-methylene-malonic acid with 1,2,2,6,6-pentamethyl-4-hydroxypiperidine, poly[methylpropyl-3-oxy-4-(2,2,6,6-tetramethyl-4-piperidyl)]siloxane, reaction product of maleic acid anhydride-α-olefin-copolymer with 2,2,6,6-tetramethyl-4-aminopiperidine or 1,2,2,6,6-pentamethyl-4-aminopiperidine.

The sterically hindered amine may also be one of the compounds described in U.S. Pat. No. 5,980,783, the relevant parts of which are hereby incorporated by reference, that is compounds of component I-a), I-b), I-c), I-d), I-e), I-f), I-g), I-h), I-i), I-j), I-k) or I-l), in particular the light stabilizer 1-a-1, 1-a-2, 1-b-1, 1-c-1, 1-c-2, 1-d-1, 1-d-2, 1-d-3, 1-e-1, 1-f-1, 1-g-1, 1-g-2 or 1-k-1 listed on columns 64-72 of said U.S. Pat. No. 5,980,783.

The sterically hindered amine may also be one of the compounds described in EP 782994, for example compounds as described in claims 10 or 38 or in Examples 1-12 or D-1 to D-5 therein.

2.7. Sterically hindered amines substituted on the N-atom by a hydroxy-substituted alkoxy group, for example compounds such as 1-(2-hydroxy-2-methylpropoxy)-4-octadecanoyloxy-2,2,6,6-tetramethylpiperidine, 1-(2-hydroxy-2-methylpropoxy)-4-hexadecanoyloxy-2,2,6,6-tetramethylpiperidine, the reaction product of 1-oxyl-4-hydroxy-2,2,6,6-tetramethylpiperidine with a carbon radical from t-amylalcohol, 1-(2-hydroxy-2-methylpropoxy)-4-hydroxy-2,2,6,6-tetramethylpiperidine, 1-(2-hydroxy-2-methylpropoxy)-4-oxo-2,2,6,6-tetramethylpiperidine, bis(1-(2-hydroxy-2-methylpropoxy)-2,2,6,6-tetramethylpiperidin-4-yl) sebacate, bis(1-(2-hydroxy-2-methylpropoxy)-2,2,6,6-tetramethylpiperidin-4-yl) adipate, bis(1-(2-hydroxy-2-methylpropoxy)-2,2,6,6-tetramethylpiperidin-4-yl) succinate, bis(1-(2-hydroxy-2-methylpropoxy)-2,2,6,6-tetramethylpiperidin-4-yl) glutarate and 2,4-bis{N-[1-(2-hydroxy-2-methylpropoxy)-2,2,6,6-tetramethylpiperidin-4-yl]-N-butylamino}-6-(2-hydroxyethylamino)-s-triazine.

2.8. Oxamides, for example 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, mixtures of o- and p-methoxy-disubstituted oxanilides and mixtures of o- and p-ethoxy-disubstituted oxanilides.

2.9. Tris-aryl-o-hydroxyphenyl-s-triazines, for example known commercial tris-aryl-o-hydroxyphenyl-s-triazines and triazines as disclosed in, WO 96/28431 and U.S. Pat. Nos. 3,843,371; 4,619,956; 4,740,542; 5,096,489; 5,106,891; 5,298,067; 5,300,414; 5,354,794; 5,461,151; 5,476,937; 5,489,503; 5,543,518; 5,556,973; 5,597,854; 5,681,955; 5,726,309; 5,736,597; 5,942,626; 5,959,008; 5,998,116; 6,013,704; 6,060,543; 6,187,919; 6,242,598 and 6,255,483, for example 4,6-bis-(2,4-dimethylphenyl)-2-(2-hydroxy-4-octyloxyphenyl)-s-triazine, Cyasorb® 1164, Cytec Corp, 4,6-bis-(2,4-dimethylphenyl)-2-(2,4-dihydroxyphenyl)-s-triazine, 2,4-bis(2,4-dihydroxyphenyl)-6-(4-chlorophenyl)-s-triazine, 2,4-bis[2-hydroxy-4-(2-hydroxyethoxy)phenyl]-6-(4-chlorophenyl)-s-triazine, 2,4-bis[2-hydroxy-4-(2-hydroxy-4-(2-hydroxyethoxy)phenyl]-6-(2,4-dimethylphenyl)-s-triazine, 2,4-bis[2-hydroxy-4-(2-hydroxyethoxy)phenyl]-6-(4-bromophenyl)-s-triazine, 2,4-bis[2-hydroxy-4-(2-acetoxyethoxy)phenyl]-6-(4-chlorophenyl)-s-triazine, 2,4-bis(2,4-dihydroxyphenyl)-6-(2,4-dimethylphenyl)-s-triazine, 2,4-bis(4-biphenylyl)-6-(2-hydroxy-4-octyloxycarbonylethylideneoxyphenyl)-s-triazine, 2-phenyl-4-[2-hydroxy-4-(3-sec-butyloxy-2-hydroxypropyloxy)phenyl]-6-[2-hydroxy-4-(3-sec-amyloxy-2-hydroxypropyloxy)phenyl]-s-triazine, 2,4-bis(2,4-dimethylphenyl)-6-[2-hydroxy-4-(3-benzyloxy-2-hydroxypropyloxy)phenyl]-s-triazine, 2,4-bis(2-hydroxy-4-n-butyloxyphenyl)-6-(2,4-di-n-butyloxyphenyl)-s-triazine, 2,4-bis(2,4-dimethylphenyl)-6-[2-hydroxy-4-(3-nonyloxy*-2-hydroxypropyloxy)-5-α-cumylphenyl]-s-triazine (* denotes a mixture of octyloxy, nonyloxy and decyloxy groups), methylenebis-{2,4-bis(2,4-dimethylphenyl)-6-[2-hydroxy-4-(3-butyloxy-2-hydroxypropoxy)phenyl]-s-triazine}, methylene bridged dimer mixture bridged in the 3:5′, 5:5′ and 3:3′ positions in a 5:4:1 ratio, 2,4,6-tris(2-hydroxy-4-isooctyloxycarbonylisopropylideneoxy-phenyl)-s-triazine, 2,4-bis(2,4-dimethylphenyl)-6-(2-hydroxy-4-hexyloxy-5-α-cumylphenyl)-s-triazine, 2-(2,4,6-trimethylphenyl)-4,6-bis[2-hydroxy-4-(3-butyloxy-2-hydroxypropyloxy)phenyl]-s-triazine, 2,4,6-tris[2-hydroxy-4-(3-sec-butyloxy-2-hydroxypropyloxy)phenyl]-s-triazine, mixture of 4,6-bis-(2,4-dimethylphenyl)-2-(2-hydroxy-4-(3-dodecyloxy-2-hydroxypropoxy)-phenyl)-s-triazine and 4,6-bis-(2,4-dimethylphenyl)-2-(2-hydroxy-4-(3-tridecyloxy-2-hydroxypropoxy)-phenyl)-s-triazine, Tinuvin® 400, Ciba Specialty Chemicals Corp., 4,6-bis-(2,4-dimethylphenyl)-2-(2-hydroxy-4-(3-(2-ethylhexyloxy)-2-hydroxypropoxy)-phenyl)-s-triazine and 4,6-diphenyl-2-(4-hexyloxy-2-hydroxyphenyl)-s-triazine.

3. Metal deactivators, for example N,N′-diphenyloxamide, N-salicylal-N′-salicyloyl hydrazine, N,N′-bis(salicyloyl) hydrazine, N,N′-bis(3,5-di-tert-butyl-4-hydroxyphenylpropionyl) hydrazine, 3-salicyloylamino-1,2,4-triazole, bis(benzylidene)oxalyl dihydrazide, oxanilide, isophthaloyl dihydrazide, sebacoyl bisphenylhydrazide, N,N′-diacetyladipoyl dihydrazide, N,N′-bis(salicyloyl)oxalyl dihydrazide, N,N′-bis(salicyloyl)thiopropionyl dihydrazide.

4. Phosphites and phosphonites, for example triphenyl phosphite, diphenyl alkyl phosphites, phenyl dialkyl phosphites, tris(nonylphenyl) phosphite, trilauryl phosphite, trioctadecyl phosphite, distearyl pentaerythritol diphosphite, tris(2,4-di-tert-butylphenyl) phosphite, diisodecyl pentaerythritol diphosphite, bis(2,4-di-tert-butylphenyl) pentaerythritol diphosphite, bis(2,6-di-tert-butyl-4-methylphenyl)-pentaerythritol diphosphite, diisodecyloxypentaerythritol diphosphite, bis(2,4-di-tert-butyl-6-methylphenyl)pentaerythritol diphosphite, bis(2,4,6-tris(tert-butylphenyl)pentaerythritol diphosphite, tristearyl sorbitol triphosphite, tetrakis(2,4-di-tert-butylphenyl) 4,4′-biphenylene diphosphonite, 6-isooctyloxy-2,4,8,10-tetra-tert-butyl-dibenzo[d,f][1,3,2]dioxaphosphepin, 6-fluoro-2,4,8,10-tetra-tert-butyl-12-methyl-dibenzo[d,g][1,3,2]dioxaphosphocin, bis(2,4-di-tert-butyl-6-methylphenyl) methyl phosphite, bis(2,4-di-tert-butyl-6-methylphenyl) ethyl phosphite, 2,2′,2″-nitrilo[triethyltris(3,3′,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-diyl)phosphite.

Especially preferred are the following phosphites:

Tris(2,4-di-tert-butylphenyl) phosphite, tris(nonylphenyl) phosphite,

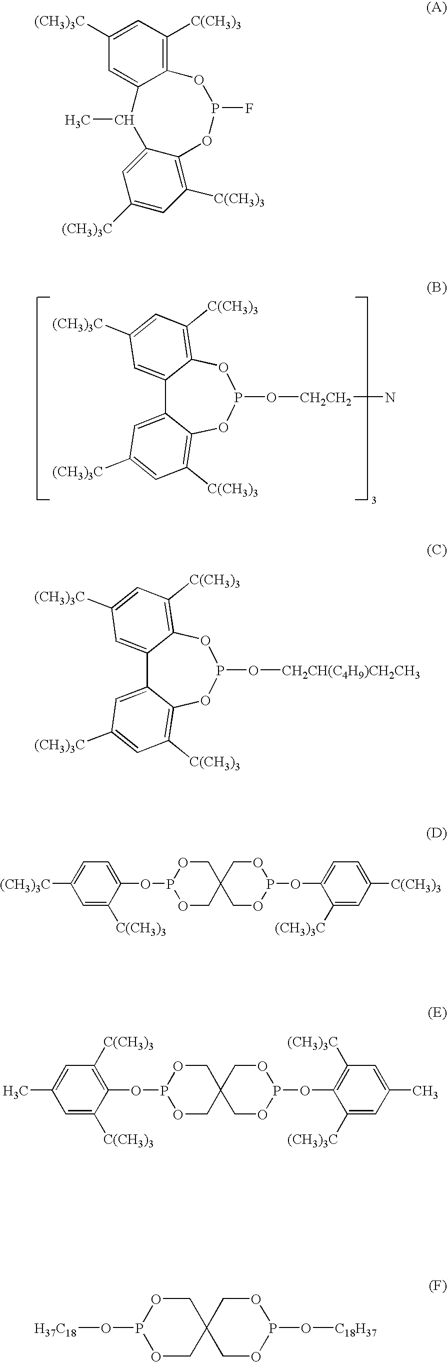

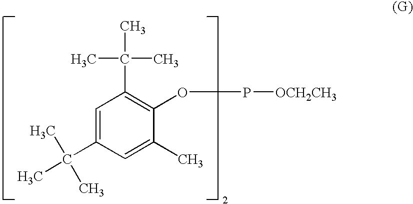

5. Hydroxylamines, for example N,N-dibenzylhydroxylamine, N,N-diethylhydroxylamine, N,N-dioctylhydroxylamine, N,N-dilaurylhydroxylamine, N,N-ditetradecylhydroxylamine, N,N-dihexadecylhydroxylamine, N,N-dioctadecylhydroxylamine, N-hexadecyl-N-octadecylhydroxylamine, N-heptadecyl-N-octadecylhydroxylamine, N-methyl-N-octadecylhydroxylamine and the N,N-dialkylhydroxylamine derived from hydrogenated tallow amine.

6. Nitrones, for example N-benzyl-α-phenylnitrone, N-ethyl-α-methylnitrone, N-octyl-α-heptylnitrone, N-lauryl-α-undecylnitrone, N-tetradecyl-α-tridcylnitrone, N-hexadecyl-α-pentadecylnitrone, N-octadecyl-α-heptadecyinitrone, N-hexadecyl-α-heptadecylnitrone, N-ocatadecyl-α-pentadecylnitrone, N-heptadecyl-α-heptadecylnitrone, N-octadecyl-α-hexadecylnitrone, N-methyl-α-heptadecylnitrone and the nitrone derived from N,N-dialkylhydroxylamine derived from hydrogenated tallow amine.

7. Amine oxides, for example amine oxide derivatives as disclosed in U.S. Pat. Nos. 5,844,029 and 5,880,191, didecyl methyl amine oxide, tridecyl amine oxide, tridodecyl amine oxide and trihexadecyl amine oxide.

8. Benzofuranones and indolinones, for example those disclosed in U.S. Pat. Nos. 4,325,863, 4,338,244, 5,175,312, 5,216,052, 5,252,643; DE-A-4316611; DE-A-431 6622; DE-A-4316876; EP-A-0589839 or EP-A-0591102 or 3-[4-(2-acetoxyethoxy)phenyl]-5,7-di-tert-butyl-benzofuran-2-one, 5,7-di-tert-butyl-3-[4-(2-stearoyloxyethoxy)phenyl]-benzofuran-2-one, 3,3′-bis[5,7-di-tert-butyl-3-(4-[2-hydroxyethoxy]phenyl)benzofuran-2-one], 5,7-di-tert-butyl-3-(4-ethoxyphenyl)benzofuran-2-one, 3-(4-acetoxy-3,5-dimethylphenyl)-5,7-di-tert-butyl-benzofuran-2-one, 3-(3,5-dimethyl-4-pivaloyloxyphenyl)-5,7-di-tert-butyl-benzofuran-2-one, 3-(3,4-dimethylphenyl)-5,7-di-tert-butyl-benzofuran-2-one, and 3-(2,3-dimethylphenyl)-5,7-di-tert-butyl-benzofuran-2-one.

9. Thiosynergists, for example dilauryl thiodipropionate or distearyl thiodipropionate.

10. Peroxide scavengers, for example esters of β-thiodipropionic acid, for example the lauryl, stearyl, myristyl or tridecyl esters, mercaptobenzimidazole or the zinc salt of 2-mercaptobenzimidazole, zinc dibutyldithiocarbamate, dioctadecyl disulfide, pentaerythritol tetrakis(β-dodecylmercapto)propionate.

11. Polyamide stabilizers, for example copper salts in combination with iodides and/or phosphorus compounds and salts of divalent manganese.

12. Basic co-stabilizers, for example melamine, polyvinylpyrrolidone, dicyandiamide, triallyl cyanurate, urea derivatives, hydrazine derivatives, amines, polyamides, polyurethanes, alkali metal salts and alkaline earth metal salts of higher fatty acids, for example, calcium stearate, zinc stearate, magnesium behenate, magnesium stearate, sodium ricinoleate and potassium palmitate, antimony pyrocatecholate or zinc pyrocatecholate.

13. Nucleating agents, for example inorganic substances such as talcum, metal oxides such as titanium dioxide or magnesium oxide, phosphates, carbonates or sulfates of, preferably, alkaline earth metals; organic compounds such as mono- or polycarboxylic acids and the salts thereof, e.g. 4-tert-butylbenzoic acid, adipic acid, diphenylacetic acid, sodium succinate or sodium benzoate; polymeric compounds such as ionic copolymers (ionomers).

14. Fillers and reinforcing agents, for example calcium carbonate, silicates, glass fibres, glass bulbs, asbestos, talc, kaolin, mica, barium sulfate, metal oxides and hydroxides, carbon black, graphite, wood flour and flours or fibers of other natural products, synthetic fibers.

15. Dispersing Agents, such as polyethylene oxide waxes or mineral oil.

16. Other additives, for example plasticizers, lubricants, emulsifiers, pigments, dyes, optical brighteners, rheology additives, catalysts, flow-control agents, slip agents, crosslinking agents, crosslinking boosters, halogen scavengers, smoke inhibitors, flameproofing agents, antistatic agents, clarifiers such as substituted and unsubstituted bisbenzylidene sorbitols, benzoxazinone UV absorbers such as 2,2′-p-phenylene-bis(3,1-benzoxazin-4-one), Cyasor® 3638 (CAS# 18600-59-4), and blowing agents.

The sterically hindered alkoxyamines and hydroxy substituted alkoxyamines of the present invention are prepared according to known methods, with the additional step of incorporating a water compatible or water soluble side chain.

The preparation of sterically hindered alkoxyamine stabilizers, also known as N-alkoxy hindered amines and NOR hindered amines or NOR hindered amine light stabilizers or NOR HALS, is disclosed for example in U.S. Pat. Nos. 5,004,770 and 5,096,950, the relevant disclosures of which are hereby incorporated by reference.

The preparation of sterically hindered hydroxy substituted alkoxyamine stabilizers, also known as hindered hydroxyalkoxyamine stabilizers, N-hydroxyalkoxy hindered amines, or NORol HALS, is disclosed for example in U.S. Pat. Nos. 6,271,377, 6,392,041 and 6,376,584, the relevant disclosures of which are hereby incorporated by reference.

The following non-limiting examples further illustrate the present invention.

Test compounds:

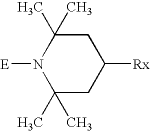

when E is 2-hydroxycyclohexyloxy or 2-hydroxy-2-methylpropoxy,

Rₓ is selected from the group consisting of

—NH₂⁺CH₂CH₂OH Cl⁻, —NHCH₂CH₂OH, —NH₃⁺⁻OAc, ═NOH, —NHCH(CH₃)COO⁻K⁺, —NHCH₂CH₂N(CH₃)₂⁺⁻OAc, —NHCH₂CH₂SO₃⁻K⁺, —NHCH(COO⁻K⁺)CH₂CH₂SCH₃, —NHCH₂COO⁻K⁺, —NHCOCH₂OH, —NHCOCH₂NHCOCH₃, —NHCH₂CH₂CH₂SO₃H, —OCH₂CH₂OH, —OCH(CH₃)COO⁻K⁺, —OCH₂CH₂N(CH₃)₂⁺⁻OAc, —OCH₂CH₂SO₃^{-K}⁺, —OCH(COO⁻K⁺)CH₂CH₂SCH₃, —OCH₂COO⁻K⁺, —OCOCH₂OH, —OCOCH₂NHCOCH₃ and —OCH₂CH₂CH₂SO₃H,

when E is benzyloxy, methoxy, propoxy, hexyloxy, heptyloxy, octyloxy or cyclohexyloxy,

Rₓ is selected from the group consisting of

—NH₂⁺CH₂CH₂OH Cl⁻, —NH₃⁺⁻OAc, ═NOH, —NHCH(CH₃)COO⁻K⁺, —NHCH₂CH₂N(CH₃)₂⁺⁻OAc, —NHCH₂CH₂SO₃⁻K⁺, —NHCH(COO⁻K⁺)CH₂CH₂SCH₃, —NHCH₂COO⁻K⁺, —OCH(CH₃)COO⁻K⁺, —OCH₂CH₂N(CH₃)₂⁺⁻OAc, —OCH₂CH₂SO₃⁻K⁺, —OCH(COO⁻K⁺)CH₂CH₂SCH₃ and —OCH₂COO⁻K⁺, and

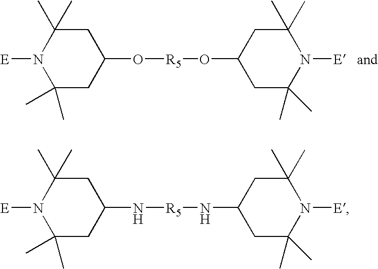

where E and E′ are 2-hydroxycyclohexyloxy, 2-hydroxy-2-methylpropoxy, benzyloxy, methoxy, propoxy, hexyloxy, heptyloxy, octyloxy or cyclohexyloxy, and

where R₅ comprises repeating units selected from the group consisting of

—(OCH₂CH₂)—, —(OCH₂CH₂(CH₃))—, —(CH₂CHCOOH)—, —(CH₂C(CH₃)COOH)—, —(CH₂CHCOOCH₃)—, —(NHCH₂CH₂)—, —(CH₂CHOH)—, —(CH₂CHCONH₂)— and —(CH₂CH(NHCOH))—.

The following Examples more particularly point out the aspects of the present invention.

The Examples are as follows:

Compounds: Examples 1-18, 22, 37, 39, 43, 52-54, 57, 59-60, 62 and 102-111.

Ink Jet Media: Examples 23, 29-36 and 38.

Solubility: Examples 44-47.

Shampoo Formulations: Examples 48-49.

Mouthwash Formulations: Examples 50-51.

Coatings: Examples 101,112-115.

Inks: Examples 19-20 and 24.

Further Ink Jet Media: Examples 21, 25-28 and 40-42.

Further Compounds: Examples 55-56, 58 and 61.

Further Shampoo Formulations: Examples 63-69 and 72.

Body care products, household products, textile and fabrics: Examples 70-71 and 73-76.

Polymer Formulations (compositions, fibers, plaques, combination with flame retardants, etc.): Examples 77-99, 120-125, 144-146, 149-167 and 173-174.

Further Coatings: Examples 100, 116-119, 126-141 and 147-148.

Photographic Compositions: Examples 142-143.

Photocured Inks: Examples 168-169.

Photocured Coatings: Examples 170-172.

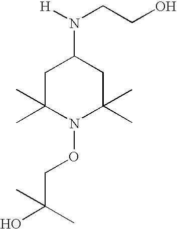

Example 39 (8.4 g, 0.035 mole), ethanolamine (3 g, 0.05 mole), and catalyst (10% Pd on C, 2 g, Engelhard Corp.) are added to 10 mL of isopropanol in 100 mL of ethanol. The reactor is pressured to 45 psig with hydrogen while heating to 60C. After a reaction time of two hours, the reactor is vented and catalyst is removed by filtration. The solvent is distilled off and the crude product is crystallized from 50 mL of ethyl acetate. The title compound is obtained (5.23 g, 52% yield) as clear white plates with a melting point of 130-132C. whose structure is consistent with HNMR.

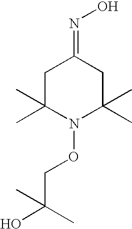

Example 39 (3.36 g, 0.14 mole) and hydroxylamine hydrochloride (1.39 g, 0.02 mole) are dissolved in a solution of 50 mL of water and 50 mL of ethanol. The solution is neutralized to pH=10 with 2N aqueous sodium hydroxide solution. The solution is refluxed for 5 hours after which the solvent is distilled off and the residue is crystallized from ethyl acetate. After drying to constant weight, the title compound is obtained (3.11 g, 86% yield) as a white solid with a melting point of 131-134C. whose structure is consistent with HNMR.

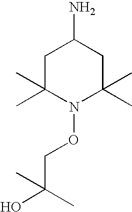

Example 2 (2.44 g, 0.0094 mole) is added to 50 mL of absolute ethanol. Sodium spheres (4 g, 0.17 mole) are added portionwise over thirty minutes after which the mixture is refluxed for one hour. The solution is poured into 200 mL of water and extracted thrice with 100 mL of methylene chloride. The combined organic layer is washed twice with 25 mL of water, dried over magnesium sulfate, and the solvent is distilled off. The title compound is obtained (2.3 g, 100% yield) as a white solid with a melting point of 110-114C. whose structure is consistent with HNMR.

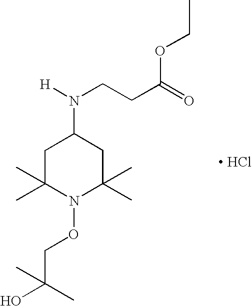

Example 39 (5.28 g, 0.022 mole), beta-alanine ethylester hydrochloride (3.84 g, 0.025 mole), and catalyst (PtO₂, 1.0 g, Engelhard Corp.) are added to 75 mL of absolute ethanol. The reactor is pressured up to 45 psig with hydrogen while heating to 60C. After four hours, the reactor is vented and the catalyst is removed by filtration. Sixty-five mL of ethanol was distilled yielding a precipitate in the remaining ethanol. After filtration, the precipitate is recrystallized from methanol. The title compound is obtained as a white solid with a melting point of 215-220C. whose structure is consistent with HNMR.

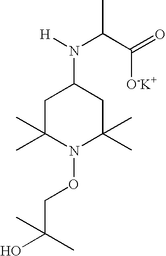

Example 39 (5.28 g, 0.022 mole), I-alanine (2.23 g, 0.025 mole), potassium hydroxide (1.4 g, 0.025 mole), and catalyst (PtO₂, 0.5 g, Engelhard Corp.) are added to 50 mL of absolute methanol. The reactor is pressured up to 45 psig with hydrogen while heating to 60C. After four hours, the reactor is vented and the catalyst is removed by filtration. The solvent is removed by distillation and the residue is dried in vacuo, which crystallizes during drying. The title compound is obtained (7 g, 100% yield) as a white glassy solid with a melting point of 65-70C. whose structure is consistent with HNMR.

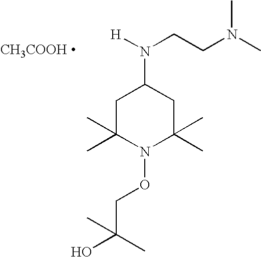

Example 39 (5.28 g, 0.022 mole), N,N-ethylenediamine (2.2 g, 0.025 mole), and catalyst (PtO₂, 0.5 g, Engelhard Corp.) are added to 75 mL of absolute methanol. The reactor is pressured up to 45 psig with hydrogen while heating to 60C. After one hour, the reactor is vented and the catalyst is removed by filtration. The solvent is removed by distillation and the residue is dissolved in 10 mL of acetonitrile and titrated with 1.5 g of glacial acetic acid. The solvent is removed by distillation and the residue is dried overnight in a vacuum oven. The title compound is obtained (7.69 g) as a white solid with a melting point of 72-74C. whose structure is consistent with HNMR.

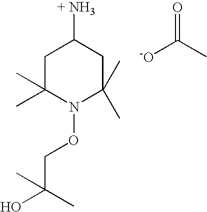

Example 3 (2.55 g, 0.01 mole) is dissolved in 100 mL of diethylether. To this solution is added 0.56 g of glacial acetic acid. A solid, which forms immediately, is filtered off, washed with diethylether, and dried to constant weight in a vacuum oven. The title compound is obtained (1.7 g, 56% yield) as a white solid with a melting point of 200-202C. whose structure is consistent with HNMR.

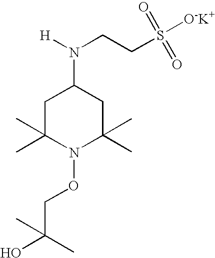

Example 39 (5.28 g, 0.022 mole), taurine (3.12 g, 0.025 mole), and catalyst (PtO₂, 0.5 g, Engelhard Corp.) are added to 50 mL of methanol and 25 mL of 1M methanolic potassium hydroxide. The reactor is pressured to 45 psig with hydrogen while heating to 60C. After 4 hours, the reactor is vented and the catalyst is filtered off. The filtrate is subjected to vacuum distillation yielding a clear residue that solidifies upon standing. The title compound is obtained (7.7 g, 89.5% yield) as a glassy white solid with a melting point of 158-162C. whose structure is consistent with HNMR.

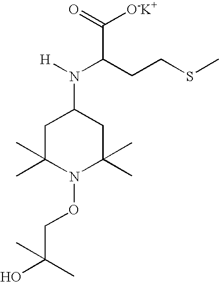

Example 39 (5.28 g, 0.022 mole), methionine (3.72 g, 0.025 mole), and catalyst (PtO₂, 0.5 g, Engelhard Corp.) are added to 50 mL of methanol and 25 mL of 1M methanolic potassium hydroxide. The reactor is pressured to 45 psig with hydrogen while heating to 60C. After 3 hours, the reactor is vented and the catalyst is filtered off. The filtrate is subjected to vacuum distillation yielding a clear residue that solidifies upon standing. The title compound is obtained (9.45 g) as a white solid with a melting point of 108-112C. whose structure is consistent with HNMR.

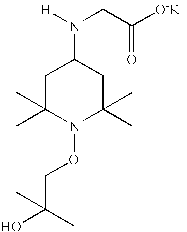

Example 39 (5.28 g, 0.022 mole), glycine (1.88 g, 0.025 mole), and catalyst (PtO₂, 0.5 g, Engelhard Corp.) are added to 50 mL of methanol and 25 mL of 1M methanolic potassium hydroxide. The reactor is pressured to 45 psig with hydrogen while heating to 60C. After 2 hours, the reactor is vented and the catalyst is filtered off. The filtrate is subjected to vacuum distillation yielding a clear residue that solidifies upon standing. The title compound is obtained (7.87 g) as a white solid with a melting point of 94-98C. whose structure is consistent with HNMR.

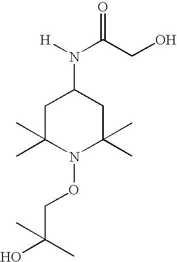

Example 3 (6.11 g, 0.025 mole) and methyl glycolate (4.5 g, 0.05 mole) are added to 75 mL of xylene. Under a nitrogen atmosphere, the solution is heated to 120C. and held there for 18 hours. The solvent is removed by distillation and the residue is chromatographed on silica gel using a hexane:ethyl acetate gradient. The appropriate fractions are combined and the solvent is removed by distillation yielding a clear residue that solidifies upon standing. The title compound is obtained (1.51 g, 20% yield) as a white solid with a melting point of 153-154C. whose structure is consistent with HNMR.

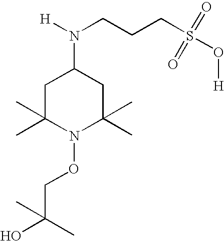

Synthetic Reference: I. Ismail, J. Serb. Chem. Soc. 57(7), 415-420 (1992)

Example 3 (4.88 g, 0.02 mole) and 1,1-propanesultone (2.44 g, 0.02 mole) are added to 60 mL of 2-butanol. Under a nitrogen atmosphere, the solution is heated to reflux for 30 minutes. The precipitate is filtered at ambient temperature, washed with 2-butanol, and dried to constant weight in a vacuum oven. The title compound is obtained (4.45 g, 60.5% yield) as a white solid that decomposes upon melting at 290C. whose structure is consistent with HNMR.

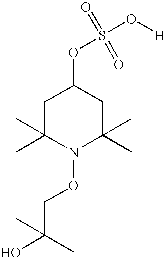

4-Hydroxy-1-(2-hydroxy-2-methylpropoxy)-2,2,6,6-tetramethylpiperidine (4.9 g, 0.02 mole) is dissolved in 200 mL of 1,2-dichloroethane and cooled to 5C. To this solution is added drop wise chlorosulfonic acid (1.32 mL, 0.02 mole) dissolved in 25 mL of 1,2-dichloroethane. The solution is left stirring overnight allowing the temperature to rise to ambient temperature. The solvent is removed by distillation and replaced with ethanol. The solution is clarified and the ethanol is removed by distillation. The title compound is obtained as a viscous clear resin whose structure is consistent with HNMR.

Analysis

HNMR (CD₃OD): δ 1.29 (s, 6H), 1.56 (s, 6H), 1.57 (s, 6H), 1.82 (dd, 2H), 2.16 (ddd, 2H), 4.16 (tt, 1H), 4.17 (s, 2H)

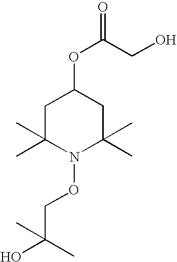

4-Hydroxy-1-(2-hydroxy-2-methylpropoxy)-2,2,6,6-tetramethylpiperidine (12.25 g, 0.05 mole), methyl glycolate (6.75 g, 0.075 mole), and Tyzor TBT (0.5 mL, 0.0013 mole) are dissolved in 250 mL of dry toluene. The solution is heated to reflux and allowed to reflux for 5 hours. The solution is cooled to 100C. at which time 5 mL of water is added. The water is removed, the toulene layer is clarified, and the touluene is removed by distillation. The title compound is obtained (17.56 g) as a light orange oil with an assay of 87% as judged by gas chromatography whose structure is consistent with HNMR.

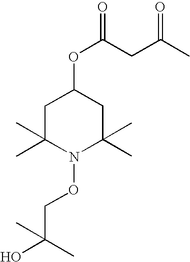

4-Hydroxy-1-(2-hydroxy-2-methylpropoxy)-2,6,6-tetramethylpiperidine (12.25 g, 0.05 mole, ethylacetoacetate (7.65 mL, 0.06 mole), and lithium t-butoxide (0.5 g, 0.006 mole) are dissolved in 250 mL of dry toluene. The solution is heated to reflux using a Dean Stark trap and allowed to reflux for 6 hours. The solution is cooled to 100C. at which time 5 mL of water is added to destroy the catalyst. The water is removed, the toluene layer is clarified, and the toulene is removed by distillation. The title compound is obtained (16.7 g) as a light orange oil whose structure is consistent with HNMR.

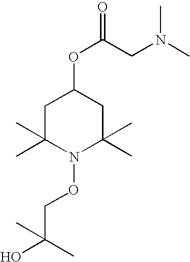

4-Hydroxy-1-(2-hydroxy-2-methylpropoxy)-2,6,6-tetramethylpiperidine (12.25 g, 0.05 mole, N,N-dimethylglycine ethyl ester (13 mL, 0.09 mole), and lithium t-butoxide (0.2 g, 0.0025 mole) are dissolved in 150 mL of dry toulene. The solution is heated to reflux using a Dean Stark trap and allowed to reflux for 5 hours. The solution is cooled to 100C. at which time 5 mL of water is added to destroy the catalyst. The water is removed, the toluene layer is clarified, and the toluene is removed by distillation. The title compound is obtained (15.58 g, 94% yield) as a light yellow-orange oil with an assay of 93.2% as judged by gas chromatography whose structure is consistent with HNMR.

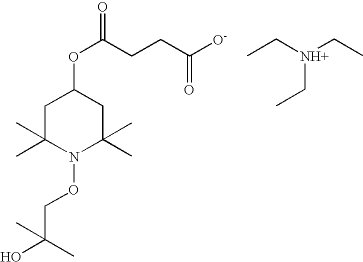

4-Hydroxy-1-(2-hydroxy-2-methylpropoxy)-2,2,6,6-tetramethylpiperidine (12.25 g, 0.05 mole), succinic acid monomethylester (6.6 g, 0.05 mole), triethylamine (5.05 g, 0.05 mole), and lithium t-butoxide (0.4 g, 0.005 mole) are dissolved in 200 mL of dry toluene. The solution is heated to reflux using a Dean Stark trap and allowed to reflux for 8 hours. The solution is cooled to 100C. at which time 5 mL of water is added to destroy the catalyst. The water is removed, the toluene layer is clarified, and the toluene is removed by distillation. The title compound is obtained as a light yellow-orange oil (21.9 g) with an assay of 21% (remaining material is unreacted starting hindered amine) as judged by gas chromatography whose structure is consistent with HNMR.

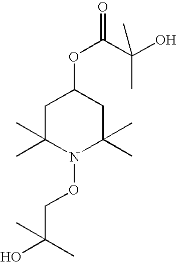

4-Hydroxy-1-(2-hydroxy-2-methylpropoxy)-2,2,6,6-tetramethylpiperidine (12.25 g, 0.05 mole), methyl 4-hydroxyisobutyrate (7.08 g, 0.06 mole), and titanium isopropoxide (0.75 mL, 0.0025 mole) are dissolved in 250 mL of dry toluene. The solution is heated to reflux using a Dean Stark trap and allowed to reflux for 18 hours. The solution is cooled to 100C. at which time 5 mL of water is added to destroy the catalyst. The water is removed, the toluene layer is clarified, and the toluene is removed by distillation. The title compound is obtained (21.41 g) as a light-yellow oil with an assay of 79% as judged by gas chromatography whose structure is consistent with HNMR.

### EXAMPLE 19

### Ink Jet Ink

An ink-jet ink is prepared by dissolving 2 g of dye in 20 g of diethylene glycol and 78 g of deionized water. The dye used is Acid red 52. The stabilizer is weighed in an amount of 0.15 g into a test tube and dissolved in 2.85 g of ink. The obtained ink is filtered and transferred into an emptied and carefully cleaned cartridge of a Deskjet 510 printer (Hewlett-Packard). A stepped image is then printed onto plain paper (sihl+eika). The produced print is left to dry at 50° C. under vacuum for two hours and thereafter irradiated behind a 5 mm thick window glass in an Atlas Ci-35 light fading device equipped with a Xenon lamp. The Atlas device is operated at 43° C., 50%RH without dark cycles and the light intensity is 461 W/m² (300-800 nm). The color density of each step is measured before and after exposure using a MacBeth TR 924 densitometer. The compounds according to this invention are able to improve substantially the light fastness of the ink-jet print.

### EXAMPLE 20

Magenta and Yellow inks are extracted from an Hewlett-Packard three-color cartridge (HP C3823D). The stabilizer is weighed in an amount of 0.15 g into a test tube and dissolved in 2.85 g of either the magenta or yellow ink. The obtained ink is filtered and transferred into an emptied and carefully cleaned cartridge of a Deskjet 510 printer (Hewlett-Packard). A stepped image is then printed onto plain paper (sihl+eika) or, alternatively, onto Premium Photo paper from Hewlett-Packard (item code C6040A). The produced print is left to dry at 50° C. under vacuum for two hours and thereafter irradiated behind a 5 mm thick window glass in an Atlas Ci-35 light fading device equipped with a Xenon lamp. The Atlas device is operated at 43° C., 50%RH without dark cycles and the light intensity is 461 W/m² (300-800 nm). The color density of each step is measured before and after exposure using a MacBeth TR 924 densitometer. The compounds according to this invention are able to improve the light fastness of the magenta and yellow prints.

### EXAMPLE 21

### Ink Jet Media

Canon PR-101 sheets (Canon Japan) are post-treated by casting stabilizer solutions, either aqueous or methanol, using a 75 micron wire wound coating bar. After drying at room temperature, the thus modified sheets are printed with cyan step images using a Canon BJC 8200 printer. The cyan dye used is C.I. Direct Blue 199. The obtained prints are left to dry at 50C. under vacuum for two hours and thereafter are subjected to forced airflow during four weeks. The color density is measured before and after exposure using a MacBeth TR 924 densitometer. The compounds according to this invention are able to improve the light fastness of dyes when printed onto printing media.

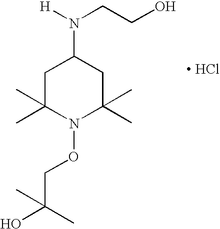

Example 1 (12.02 g as crude product, 0.042 mole) is dissolved in a mixture of diethylether and ethanol. With vigorous agitation, hydrochloric acid gas is introduced subsurface to the solution. A white insoluble solid is formed which is filtered off and dried in a vacuum oven until constant weight is reached. The title compound is obtained (2.16 g, 16% yield) as a white solid with a melting point of 217-233C. whose structure is consistent with HNMR.

### EXAMPLE 23

### Ink Jet Media

A resin-coated paper impregnated with inorganic adsorbent particles (Konica QP Photoglossy ink jet paper, Konica Corp.) is purchased. On the ink-receiving layer, a 0.8 wt % methanol solution, unless stated otherwise, of the instant compounds is applied in an amount to achieve 650-700 mg/m². The paper is allowed to dry under ambient temperature and pressure for 24 hours. Separately, test patterns (cyan) are printed on the treated sheets using a Hewlett Packard DeskJet 970 Cxi printer at 100% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting. The change in color is given by Delta E (DE) which is calculated by:

DE = [(DL*)² + (Da*)² +(Db*)²]^{1/2}

| Stabilizer | DE for cyan after 15 weeks |
|---|---|
| None | 4.29 |
| Example 11/DABCO.HCl | 4.08 |
| Example 6/DABCO | 3.09 |

DABCO.HCl is 1,4-diazabicyclo[2.2.2]octane hydrochloride salt.
DABCO is 1,4-diazabicyclo[2.2.2]octane.
Mixtures of stabilizers are in a 1:1 by weight ratio with the total stabilizer concentration added of 650-700 mg/m2.

As the above data show, compounds according to this invention improve the light fastness of ink jet prints.

### EXAMPLE 24

The instant compounds are added to an ink composition, for example as disclosed in U.S. Pat. Nos. 5,855,655 or 5,782,963, at a concentration of 2 wt % and 0.5 wt % respectively. The images printed from these stabilized inks show reduced dye fading and better image permanence.

### EXAMPLE 25

A commercial white polyethylene terephthalate sheet is coated with silica and polyvinylalcohol according to U.S. Pat. No. 6,391,440 Example 1. A methanol solution of the instant compounds is applied to this sheet in an amount equivalent to 400 mg/m². The image printed on this receiving layer shows reduced dye fading and better image permanence.

### EXAMPLE 26

A commercial white polyethylene terephthalate sheet is coated with alumina hydrate and polyvinylalcohol according to U.S. Pat. No. 6,391,440 Example 5. A methanol solution of the instant compounds is applied to this sheet in an amount equivalent to 400 mg/m². The image printed on this receiving layer shows reduced dye fading and better image permanence.

### EXAMPLE 27

A paper sheet containing alumina is prepared according to U.S. Pat. No. 6,391,440 Example 8. A methanol solution of the instant compounds is applied to this sheet in an amount equivalent to 600 mg/m2. The image printed on this receiving layer shows reduced dye fading and better image permanence.

### EXAMPLE 28

A paper sheet containing silica is prepared according to U.S. Pat. No. 5,165,973. A methanol solution of the instant compounds is applied to this sheet in an amount equivalent to 700 mg/m2. The image printed on this receiving layer shows reduced dye fading and better image permanence.

### EXAMPLE 29

A resin-coated paper impregnated with inorganic adsorbent particles (Konica QP Photoglossy ink jet paper, Konica Corp.) is purchased. On the ink-receiving layer, a 0.8 wt % methanol solution of the instant compounds is applied in an amount to achieve 650-700 mg/m². The paper is allowed to dry under ambient temperature and pressure for 24 hours. Separately, test patterns (magenta) are printed on the treated sheets using a Hewlett Packard DeskJet 970 Cxi printer at 100% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting.

| Stabilizer | DE for magenta after 15 weeks |
|---|---|
| None | 5.49 |
| Example 11/DABCO.HCl | 4.79 |
| Example 6/DABCO | 2.23 |

DABCO.HCl is 1,4-diazabicyclo[2.2.2]octane hydrochloride salt.
DABCO is 1,4-diazabicyclo[2.2.2]octane.
Mixtures of stabilizers are in a 1:1 by weight ratio with the total stabilizer concentration added of 650-700 mg/m2.

As the above data show, compounds according to this invention improve the light fastness of ink jet prints.

### EXAMPLE 30

A resin-coated paper impregnated with inorganic adsorbent particles (Konica QP Photoglossy ink jet paper, Konica Corp.) is purchased. On the ink-receiving layer, a 0.8 wt % methanol solution of the instant compounds is applied in an amount to achieve 650-700 mg/m². The paper is allowed to dry under ambient temperature and pressure for 24 hours. Separately, test patterns (yellow) are printed on the treated sheets using a Hewlett Packard DeskJet 970 Cxi printer at 100% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting.

| Stabilizer | DE for yellow after 15 weeks |
|---|---|
| None | 2.48 |
| Example 11/DABCO.HCl | 2.35 |
| Example 6/DABCO | 1.73 |

DABCO.HCl is 1,4-diazabicyclo[2.2.2]octane hydrochloride salt.
DABCO is 1,4-diazabicyclo[2.2.2]octane.
Mixtures of stabilizers are in a 1:1 by weight ratio with the total stabilizer concentration added of 650-700 mg/m2.

As the above data show, compounds according to this invention improve the light fastness of ink jet prints.

### EXAMPLE 31

A resin-coated paper impregnated with inorganic adsorbent particles (Konica QP Photoglossy ink jet paper, Konica Corp.) is purchased. On the ink-receiving layer, a 0.8 wt % methanol solution of the instant compounds is applied in an amount to achieve 650-700 mg/m², unless otherwise stated. The paper is allowed to dry under ambient temperature and pressure for 24 hours. Separately, test patterns (cyan) are printed on the treated sheets using a Hewlett Packard DeskJet 970 Cxi printer at 100% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting.

| Stabillizer | DE for cyan after 4 weeks |
|---|---|
| None | 6.19 |
| Example 22 | 5.26 |
| Example 2 | 4.69 |
| Example 22 (2×) | 4.62 |
| Example 7 | 4.27 |
| Example 1 | 4.21 |

Example 22 (2x) is done at a concentration of 1300 to 1400 mg/m2.

As the above data show, compounds according to this invention improve the light fastness of ink jet prints.

### EXAMPLE 32

A resin-coated paper impregnated with inorganic adsorbent particles (Konica QP Photoglossy ink jet paper, Konica Corp.) is purchased. On the ink-receiving layer, a 0.8 wt % methanol solution of the instant compounds is applied in an amount to achieve 650-700 mg/m², unless otherwise stated. The paper is allowed to dry under ambient temperature and pressure for 24 hours. Separately, test patterns (magenta) are printed on the treated sheets using a Hewlett Packard DeskJet 970 Cxi printer at 100% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting.

| Stabilizer | DE for magenta after 4 weeks |
|---|---|
| None | 25.73 |
| Example 22 | 25.20 |
| Example 22 (2×) | 21.96 |
| Example 7 | 20.78 |
| Example 1 | 20.23 |
| Example 7 (2×) | 19.52 |
| Example 7 (3×) | 12.99 |

Example 22 (2x) and Example 7 (2x) are done at a concentration of 1300 to 1400 mg/m2 while Example 7 (3x) is done at a concentration of 1950 to 2100 mg/m2.

As the above data show, compounds according to this invention improve the light fastness of ink jet prints.

### EXAMPLE 33

A resin-coated paper impregnated with inorganic adsorbent particles (Konica QP Photoglossy ink jet paper, Konica Corp.) is purchased. On the ink-receiving layer, a 0.8 wt % methanol solution of the instant compounds is applied in an amount to achieve 650-700 mg/m², unless stated otherwise. The paper is allowed to dry under ambient temperature and pressure for 24 hours. Separately, test patterns (yellow) are printed on the treated sheets using a Hewlett Packard DeskJet 970 Cxi printer at 50% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting.

| Stabilizer | DE for yellow after 4 weeks |
|---|---|
| None | 5.80 |
| Example 22 | 5.53 |
| Example 2 | 3.87 |

As the above data show, compounds according to this invention improve the light fastness of ink jet prints.

### EXAMPLE 34

A resin-coated paper impregnated with inorganic adsorbent particles (Konica QP Photoglossy ink jet paper, Konica Corp.) is purchased. On the ink-receiving layer, a 0.8 wt % methanol solution of the instant compounds is applied in an amount to achieve 650-700 mg/m², unless otherwise stated. The paper is allowed to dry under ambient temperature and pressure for 24 hours. Separately, test patterns (cyan) are printed on the treated sheets using a Hewlett Packard DeskJet 970 Cxi printer at 50% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting.

| Stabilizer | DE for cyan after 4 weeks |
|---|---|
| None | 7.29 |
| Example 22 | 5.98 |
| Example 7 | 5.85 |
| Example 2 | 5.53 |
| Example 1 | 5.29 |
| Example 22 (2×) | 5.11 |
| Example 7 (2×) | 4.95 |
| Example 7 (3×) | 4.34 |

Example 22 (2x) and Example 7 (2x) are done at a concentration of 1300 to 1400 mg/m2 while Example 7 (3x) is done at a concentration of 1950 to 2100 mg/m2.

As the above data show, compounds according to this invention improve the light fastness of ink jet prints.

### EXAMPLE 35

A resin-coated paper impregnated with inorganic adsorbent particles (Konica QP Photoglossy ink jet paper, Konica Corp.) is purchased. On the ink-receiving layer, a 0.8 wt % methanol solution of the instant compounds is applied in an amount to achieve 650-700 mg/m², unless otherwise stated. The paper is allowed to dry under ambient temperature and pressure for 24 hours. Separately, test patterns (magenta) are printed on the treated sheets using a Hewlett Packard DeskJet 970 Cxi printer at 50% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting.

| Stabilizer | DE for magenta after 4 weeks |
|---|---|
| None | 20.16 |
| Example 7 | 16.62 |
| Example 7 (2×) | 14.14 |
| Example 7 (3×) | 8.60 |

Example 7 (2x) is done at a concentration of 1300 to 1400 mg/m2 while Example 7 (3x) is done at a concentration of 1950 to 2100 mg/m2.

As the above data show, compounds according to this invention improve the light fastness of ink jet prints.

### EXAMPLE 36

A resin-coated paper impregnated with inorganic adsorbent particles (Konica QP Photoglossy ink jet paper, Konica Corp.) is purchased. On the ink-receiving layer, a 0.8 wt % methanol solution of the instant compounds is applied in an amount to achieve 650-700 mg/m², unless stated otherwise. The paper is allowed to dry under ambient temperature and pressure for 24 hours. Separately, test patterns (yellow) are printed on the treated sheets using a Hewlett Packard DeskJet 970 Cxi printer at 50% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting.

| Stabilizer | DE for yellow after 4 weeks |
|---|---|
| None | 4.68 |
| Example 22 | 3.15 |
| Example 2 | 1.36 |

As the above data show, compounds according to this invention improve the light fastness of ink jet prints.

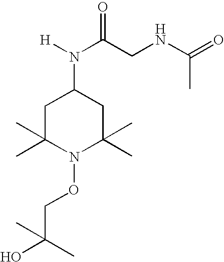

Example 3 (6.1 g, 0.025 mole) and ethyl acetamidoacetate (3.62 g, 0.025 mole) are added to 75 mL of xylene and heated to reflux. The solution is refluxed for 18 hours and then the xylene is removed by distillation. The remaining orange residue is recrystallized from methylene chloride and dried to constant weight in a vacuum oven. The title compound is obtained (2.41 g, 28% yield) as a white crystalline solid with a melting point of 77-80C. whose structure is consistent with HNMR.

### EXAMPLE 38

A resin-coated paper impregnated with inorganic adsorbent particles (Konica QP Photoglossy ink jet paper, Konica Corp.) is purchased. On the ink-receiving layer, a 0.8 wt % methanol solution of the instant compounds is applied in an amount to achieve 650-700 mg/m². The paper is allowed to dry under ambient temperature and pressure for 24 hours. Separately, test patterns (cyan and yellow) are printed on the treated sheets using a Hewlett Packard DeskJet 970 Cxi printer at 100% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting.

| Stabilizer | DE for cyan after 3 months |
|---|---|
| None | 12.40 |
| Compound A | 11.96 |
| Example 37 | 11.39 |
| Compound A/Example 37 | 10.96 |

Compound A is N,N-dibenzylhydroxylamine hydrochloride

Mixtures of stabilizers are in a 1:1 by weight ratio with the total stabilizer concentration added of 650-700 mg/m2.

| Stabilizer | DE for yellow after 3 months |
|---|---|
| None | 6.85 |
| Compound A | 5.31 |
| Example 37 | 2.96 |
| Example 37/Compound A | 1.37 |

Compound A is N,N-dibenzylhydroxylamine hydrochloride

Mixtures of stabilizers are in a 1:1 by weight ratio with the total stabilizer concentration added of 650-700 mg/m2.

As the above data show, compounds according to this invention improve the light fastness of ink jet prints.

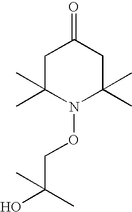

4-Hydroxy-1-(2-hydroxy-2-methylpropoxy)-2,2,6,6-tetramethylpiperidine (42.5 g, 0.17 mole), calcium hypochlorite (100 g, 0.7 mole), and resin (25 g, IRA 900 resin, ACROS) are added to 600 mL of carbon tetrachloride. After heating to 40C., the slurry is stirred for seven hours and then is stirred overnight at ambient temperature. The slurry is filtered to remove resin and salts. The resulting filtrate is washed twice with water, dried over magnesium sulfate and the solvent removed by distillation. The title compound is obtained (37.1 g, 88% yield) as a white solid with a melting point of 58-61C. whose structure is consistent with HNMR.

### EXAMPLE 40

### Ink Jet Media

A resin-coated paper impregnated with inorganic adsorbent particles (Konica QP Photoglossy ink jet paper, Konica Corp.) is purchased. On the ink-receiving layer, a 0.8 wt % methanol solution of the instant compounds and 2-(2-hydroxy-3-tert-butyl-5-methylphenyl)-2H-benzotriazole-5-sulfonic acid, sodium salt, a benzotriazole based UV absorber, is applied in an amount to achieve 650-700 mg/m². The UV absorber and the instant compounds are in a 2:1 ratio by weight. The paper is allowed to dry under ambient temperature and pressure for 24 hours. Separately, test patterns (cyan, magenta and yellow) are printed on the treated sheets using an Epson printer at 100% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting. The compounds according to this invention improve the light fastness of ink jet prints.

### EXAMPLE 41

A resin-coated paper impregnated with inorganic adsorbent particles (Konica QP Photoglossy ink jet paper, Konica Corp.) is purchased. On the ink-receiving layer, a 0.8 wt % methanol solution of the instant compounds and 2-(3-t-butyl-2-hydroxy-5-(2-(ohydroxy-octa-(ethyleneoxy)carbonyl-ethyl)-, phenyl)-2H-benzotriazole, a benzotriazole based UV absorber, is applied in an amount to achieve 650-700 mg/m². The UV absorber and the instant compounds are in a 1:1 ratio by weight. The paper is allowed to dry under ambient temperature and pressure for 24 hours. Separately, test patterns (cyan, magenta and yellow) are printed on the treated sheets using a Hewlett Packard DeskJet 970 Cxi printer at 50% print density. The obtained prints are left to dry at ambient temperature and pressure for 24 hours. Color densities and CIEL*a*b coordinates before and after exposure are measured using X-Rite 938 Spectrodensitometer. Exposures are carried out using uv lamps. The compounds according to this invention improve the light fastness of ink jet prints.

### EXAMPLE 42

A resin-coated paper impregnated with inorganic adsorbent particles (Konica QP Photoglossy ink jet paper, Konica Corp.) is purchased. On the ink-receiving layer, a 0.8 wt % methanol solution of the instant compounds and 2-(2-hydroxy-3-tert-butyl-5-methylphenyl)-2H-benzotriazole-5-sulfonic acid, sodium salt, a benzotriazole based UV absorber, is applied in an amount to achieve 650-700 mg/m². The UV absorber and the instant compounds are in a 1:1 ratio by weight. The paper is allowed to dry under ambient temperature and pressure for 24 hours. Separately, test patterns (cyan, magenta and yellow) are printed on the treated sheets using a Hewlett Packard DeskJet 970 Cxi printer at 100% print density. The obtained prints are left to dry at ambient temperature and pressure for 24 hours. Color densities and CIEL*a*b coordinates before and after exposure are measured using X-Rite 938 Spectrodensitometer. Exposures are carried out using normal office fluorescent lighting. The compounds according to this invention improve the light fastness of ink jet prints.

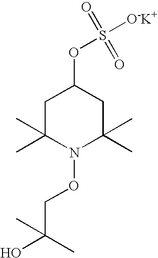

Example 13 (0.304 g, 0.001 mole) is dissolved in 2 mL of absolute ethanol. Potassium hydroxide (0.05 g, 0.001 mole) is added to the solution at which time a precipitate is formed. The precipitate is filtered and dried to constant weight in a vacuum oven. The title compound is obtained as a waxy white solid with a melting point of 115-120C. whose structure is consistent with HNMR.

### EXAMPLE 44

The solubility of the instant compounds is evaluated in polar solvent systems. A solution of 50% butyl carbitol by weight and 50% water by weight is prepared. The instant compounds are added to this solution, stirred for 30 minutes, and sampled for analysis. Samples containing bis(1-octyloxy-2,2,6,6-tetramethylpiperidin-4-yl) sebacate and 4-hydroxy-1-(2-hydroxy-2-methylpropoxy)-2,2,6,6-tetramethylpiperidine are filtered to remove any undissolved residue and all samples are analyzed by high pressure liquid chromatography or gas chromatography.

| Compound | Solubility (% by weight) |
|---|---|
| HALS A | 0.2 |
| HALS B | 13.4 |
| Example 5 | ≧15 |
| Example 10 | ≧20 |

HALS A is bis(1-octyloxy-2,2,6,6-tetramethylpiperidin-4-yl) sebacate;
HALS B is 4-hydroxy-1-(2-hydroxy-2-methylpropoxy)-2,2,6,6-tetramethylpiperidine.

This demonstrates that the instant compounds are soluble in highly polar solvents.

### EXAMPLE 45

The solubility of the instant compounds is evaluated in polar solvent systems. A solution of 50% butyl cellusolve by weight and 50% water by weight is prepared. The instant compounds are added to this solution, stirred for 30 minutes, and sampled for analysis. Samples containing bis(1-octyloxy-2,2,6,6-tetramethylpiperidin-4-yl) sebacate and 4-hydroxy-1-(2-hydroxy-2-methylpropoxy)-2,2,6,6-tetramethylpiperidine are filtered to remove any undissolved residue and all samples are analyzed by high pressure liquid chromatography or gas chromatography.

| Compound | Solubility (% by weight) |
|---|---|
| HALS A | 0.7 |
| HALS B | 15.5 |
| Example 5 | ≧18 |
| Example 10 | ≧20 |

HALS A is bis(1-octyloxy-2,2,6,6-tetramethylpiperidin-4-yl) sebacate;
HALS B is 4-hydroxy-1-(2-hydroxy-2-methylpropoxy)-2,2,6,6-tetramethylpiperidine.

This demonstrates that the instant compounds are soluble in highly polar solvents.

### EXAMPLE 46

The solubility of the instant compounds is evaluated in polar solvent systems. A solution of 25% butyl cellusolve by weight and 75% water by weight is prepared. The instant compounds are added to this solution, stirred for 30 minutes, and sampled for analysis. Samples containing bis(1-octyloxy-2,2,6,6-tetramethylpiperidin-4-yl) sebacate and 4-hydroxy-1-(2-hydroxy-2-methylpropoxy)-2,2,6,6-tetramethylpiperidine are filtered to remove any undissolved residue and all samples are analyzed by high pressure liquid chromatography or gas chromatography.

| Compound | Solubility (% by weight) |
|---|---|
| HALS A | <0.1 |
| HALS B | 13.6 |
| Example 10 | ≧18 |

HALS A is bis(1-octyloxy-2,2,6,6-tetramethylpiperidin-4-yl) sebacate.
HALS B is 4-hydroxy-1-(2-hydroxy-2-methylpropoxy)-2,2,6,6-tetramethylpiperidine.

This demonstrates that the instant compounds are soluble in highly polar solvents.

### EXAMPLE 47

The solubility of the instant compounds is evaluated in polar solvent systems. The instant compounds are added to water, stirred for 30 minutes, and sampled for analysis. Samples containing bis(1-octyloxy-2,2,6,6-tetramethylpiperidin-4-yl) sebacate and 4-hydroxy-1-(2-hydroxy-2-methylpropoxy)-2,2,6,6-tetramethylpiperidine are filtered to remove any undissolved residue and all samples are analyzed by high pressure liquid chromatography or gas chromatography.

| Compound | Solubility (% by weight) |
|---|---|
| HALS A | <0.1 |
| HALS B | 1.4 |
| Example 10 | ≧8 |

HALS A is bis(1-octyloxy-2,2,6,6-tetramethylpiperidin-4-yl) sebacate;
HALS B is 4-hydroxy-1-(2-hydroxy-2-methylpropoxy)-2,2,6,6-tetramethylpiperidine.

This demonstrates that the instant compounds are soluble in highly polar solvents.

### EXAMPLE 48

The instant compounds are added to a commercial shampoo formulation and are evaluated for their ability to reduce the amount of dye fading when the samples are exposed to fluorescent lighting. The instant compounds (0.36 g) are dissolved in 5 mL of methanol which is then added to 120 g of shampoo (Suave^{R} Natural^{R}, Fresh Mountain Strawberry) with agitation. A benzotriazole UV absorber, 2-(2-hydroxy-3-tert-butyl-5-methylphenyl)-2H-benzotriazole-5-sulfonic acid, sodium salt (UVA), is optionally added to the shampoo formulation at an equivalent concentration. The stabilized shampoo formulation is agitated for 15 minutes and put into 20 mL glass scintillation vials. These formulations are weathered under fluorescent light aging at ambient temperature. The CIEL*a*b coordinates before and after exposure are measured using X-Rite 938 Spectrodensitometer. Color change is expressed as Delta E (DE).

| Stabilizer/(Loading) | DE after 2 weeks |
|---|---|
| None | 11.74 |
| Example 17/(0.30 wt %) | 8.91 |
| Example 6/(0.30 wt %) | 7.53 |
| Example 6/UVA | 2.33 |
| (0.30 wt %/0.30 wt %) | |

The compounds according to this invention improve the light fastness of shampoo formulations.

### EXAMPLE 49

The instant compounds are added to a commercial shampoo formulation and are evaluated for their ability to reduce the amount of dye fading when the samples are exposed to fluorescent lighting. The instant compounds (0.36 g) are dissolved in 5 mL of methanol which is then added to 120 g of shampoo (Clairol^{R} Herbal Essences Shampoo) with agitation. A benzotriazole UV absorber, 2-(2-hydroxy-3-tert-butyl-5-methylphenyl)-2H-benzotriazole-5-sulfonic acid, sodium salt (UVA), is optionally added to the shampoo formulation. The stabilized shampoo formulation is agitated for 15 minutes and put into 20 mL glass scintillation vials. These formulations are weathered under fluorescent light aging at ambient temperature. The CIEL*a*b coordinates before and after exposure are measured using X-Rite 938 Spectrodensitometer. Color change is expressed as Delta E (DE).

| Stabilizer/(Loading) | DE after 2 weeks |
|---|---|
| None | 8.87 |
| Example 7/(0.30 wt %) | 3.96 |
| Example 5/(0.30 wt %) | 1.55 |
| Example 7/UVA | 0.86 |
| (0.15 wt %/0.15 wt %) | |

The compounds according to this invention improve the light fastness of shampoo formulations.

### EXAMPLE 50

The instant compounds are added to a commercial mouthwash formulation and are evaluated for their ability to reduce the amount of dye fading when the samples are exposed to fluorescent lighting. The instant compounds (0.36 g) are dissolved in 5 mL of methanol which is then added to 120 g of mouthwash (Scope^{R} Original Mint) with agitation. A benzotriazole UV absorber, 2-(2-hydroxy-3-tert-butyl-5-methylphenyl)-2H-benzotriazole-5-sulfonic acid, sodium salt (UVA), is optionally added to the mouthwash formulation. The stabilized mouthwash formulation is agitated for 15 minutes and put into 20 mL glass scintillation vials. These formulations are weathered under fluorescent light aging at ambient temperature. The CIEL*a*b coordinates before and after exposure are measured using X-Rite 938 Spectrodensitometer. Color change is expressed as Delta E (DE).

| Stabilizer/(Loading) | DE after 2 weeks |
|---|---|
| None | 6.15 |
| Example 18/(0.30 wt %) | 5.11 |
| Example 8/UVA | 4.28 |
| (0.15 wt %/0.15 wt %) | |

The compounds according to this invention improve the light fastness of mouthwash formulations.

### EXAMPLE 51

The instant compounds are added to a commercial mouthwash formulation and are evaluated for their ability to reduce the amount of dye fading when the samples are exposed to fluorescent lighting. The instant compounds (0.36 g) are dissolved in 5 mL of methanol which is then added to 120 g of mouthwash (Listerine^{R} Cool Mint) with agitation. A benzotriazole UV absorber, 2-(2-hydroxy-3-tert-butyl-5-methylphenyl)-2H-benzotriazole-5-sulfonic acid, sodium salt (UVA), is optionally added to the mouthwash formulation. The stabilized mouthwash formulation is agitated for 15 minutes and put into 20 mL glass scintillation vials. These formulations are weathered under fluorescent light aging at ambient temperature. The CIEL*a*b coordinates before and after exposure are measured using X-Rite 938 Spectrodensitometer. Color change is expressed as Delta E (DE).

| Stabilizer/(Loading) | DE after 35 days |
|---|---|
| None | 1.83 |
| Example 10/UVA | 1.21 |
| (0.15 wt %/0.15 wt %) | |

The compounds according to this invention improve the light fastness of mouthwash formulations.

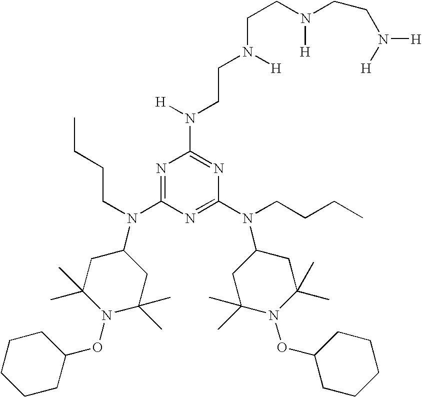

2,4-Bis[(1-cyclohexyloxy-2,2,6,6-tetramethylpiperidin-4-yl)butylamino]-6-chloro-1,3,5-triazine (20 g, 27.8 mmol) is added to DMF (150 g) at 60° C. in a stirred vessel. N4 amine (50.3 g, 288 mmol, BASF) is quickly added and the reaction mass is stirred at 60-65° C. for 1.5 hrs. Cyclohexane (150 mL) and water (200 g) are then added and the reaction mass is stirred at 50-55° C. After 15 minutes, the layers are allowed to separate. The aqueous layer is washed with cyclohexane (70 mL) at 50° C. and the cyclohexane layers are combined. The combined cyclohexane layer is washed thrice with water (100 mL/wash) at 50-55° C. The cyclohexane layer is briefly dried over anhydrous Na₂SO₄ before removing the solvent under reduced pressure. The title compound is received (24.2 g) as a light yellow oil whose structure is consistent with HNMR.

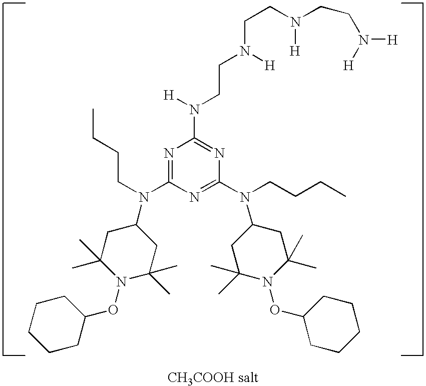

Example 52 (24.2 g, 27.8 mmol) is combined with cyclohexane (70 ml) and glacial acetic acid (1.68 g, 27.8 mmol). The solution is stirred 5 minutes and the solvent stripped under reduced pressure until constant weight. The title compound is received (25 g) as an off white solid whose structure is consistent with HNMR.

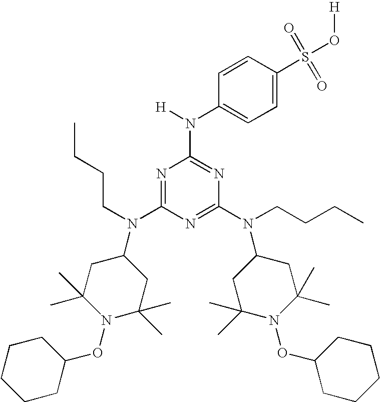

2,4-Bis[(1-cyclohexyloxy-2,2,6,6-tetramethylpiperidin-4-yl)butylamino]-6-chloro-1,3,5-triazine (13.0 g, 0.0178 mole) and N,N-dimethylformamide (12.5 g, 1.71 mole) are added to a 500 mL laboratory flask equipped with the necessary auxillary equipment. This solution is heated to 100C. at which time sulfanilic acid (14.9 g, 0.0857 mole) and anhydrous potassium carbonate (11.0 g, 0.0796 mole) are added. The temperature is raised to 145-150C. at which time sodium hydroxide (1.24 g, 0.031 mole) is added. After 15 hours at 145-150C., the reaction is cooled, diluted with water, and extracted with hexane. The lower aqueous layer is removed and further washed with hexane. The combined hexane extracts is washed thrice with water at 50C. The organic solvent is removed and the product is dried until constant weight. The title compound is received (17.1 g) as a yellow glassy solid whose structure is consistent with HNMR.

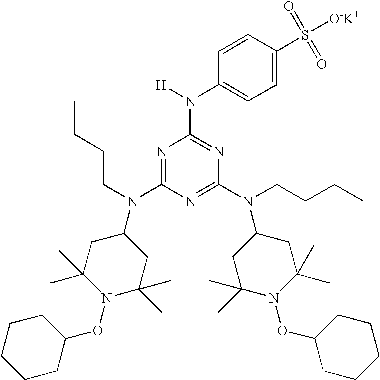

Example 54 is dissolved in 2-propanol and heated to 50C. The product is titrated with potassium hydroxide. The solvent is removed under vacuum and the resulting title compound is dried to constant weight.

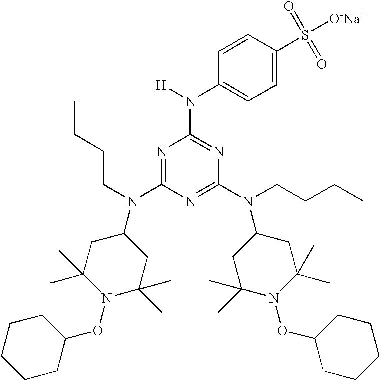

Example 54 is dissolved in 2-propanol and heated to 50C. The product is titrated with sodium hydroxide. The solvent is removed under vacuum and the resulting title compound is dried to constant weight.

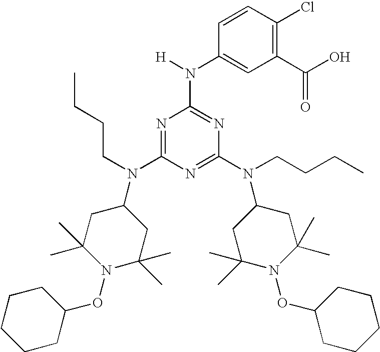

2,4-Bis[(1-cyclohexyloxy-2,2,6,6-tetramethylpiperidin-4-yl)butylamino]-6-chloro-1,3,5-triazine (13.0 g, 0.0178 mole) and N,N-dimethylformamide (12.5 g, 1.71 mole) are added to a 500 mL laboratory flask equipped with the necessary auxiliary equipment. This solution is heated to 85C. at which time 5-amino-2-chlorobenzoic acid (15 g, 0.0874 mole) and anhydrous potassium carbonate (11.0 g, 0.0796 mole) are added. The temperature is raised slowly to 120C. and held at this temperature for 15 hours. The reaction is then diluted with water and extracted with hexane. The lower aqueous layer is removed and further washed with hexane. The combined hexane extracts is washed thrice with water at 50C. The organic solvent is removed and the product is dried until constant weight. The title compound is received (14.5 g) as a light yellow glassy solid.

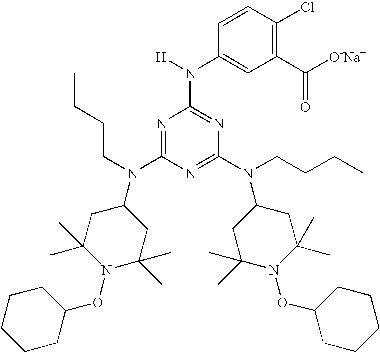

Example 57 is dissolved in 2-propanol and heated to 50C. The product is titrated with sodium hydroxide. The solvent is removed under vacuum and the resulting title compound is dried to constant weight.

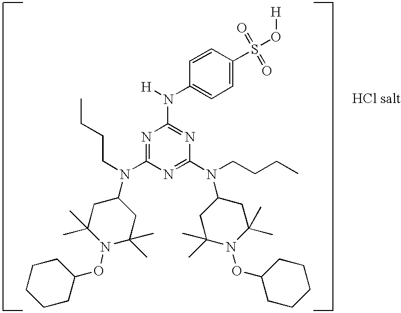

Example 54 (17.1 g, 0.0197 mole) is dissolved in anhydrous 2-propanol (150 g, 2.5 mole) and heated to 45C. Dropwise addition of anhydrous HCl gas (4.0 g, 0.11 mole) dissolved in 2-propanol (50 g, 0.83 mole) to the solution is completed in an hour and held an additional 4 hours. The solvent is removed under vacuum and the resulting compound is dried to constant weight. The title compound is received (18.6 g) as a yellow glassy solid whose structure is consistent with HNMR.

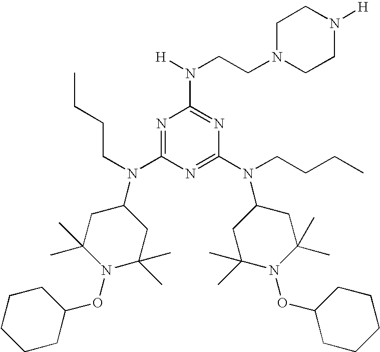

2,4-Bis[(1-cyclohexyloxy-2,2,6,6-tetramethylpiperidin-4-yl)butylamino]-6-chloro-1,3,5-triazine (40 g, 54.6 mmol) is mixed with N,N-dimethylformamide (220 g) and heated to 65° C. 1-(2-Aminoethyl)piperazine (14.5 g, 112.2 mmol) is then added quickly and the reaction is stirred at 65° C. After 2 hrs, cyclohexane (150 g) and water (65 g) are added and the mixture stirred for 10 minutes. The layers are allowed to separate and removed from the reactor. The bottom aqueous phase is returned to the pot, heated to 55° C. and water (112 g) is added. The aqueous layer is then washed twice at 55° C. with cyclohexane (50 g/each). The cyclohexane layers are combined and washed four times with water (30 g/each) at 55° C. The cyclohexane layer is dried briefly over anhydrous Na₂SO₄ before removing the solvent under reduced pressure until a constant weight is received. The title compound is received (43.4 g) as a light yellow solid whose structure is consistent with HNMR.

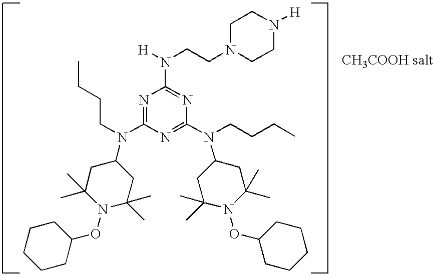

Example 60 is dissolved in 2-propanol. The product is then titrated with glacial acetic acid. The solvent is removed under vacuum and the resulting title compound is dried to constant weight.

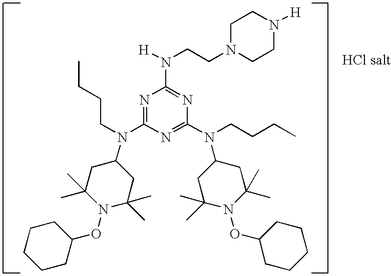

Example 60 (43.4 g, 53 mmol) is dissolved in cyclohexane (80 g). Anhydrous HCl gas (2 g, 54.8 mmol) is dissolved in 2-propanol (12 g) and added to the cyclohexane solution. The mixture is stirred for 10 minutes and the solvent then distilled under reduced pressure to a constant weight. The title compound is obtained (45 g) as a light yellow solid whose structure is consistent with HNMR.

### EXAMPLE 63

An aqueous based test formulation is prepared as follows:

| | |
|---|---|
| sodium laureth sulfate (30%, TEXAPON NSO, Cognis) | 30% |
| cocamidopropylbetaine (30%, DEHYTON K, Cognis) | 10% |
| colorant* | 0.001% |
| instant stabilizer | 0.05% |
| citric acid (10% aqueous solution) | to pH 6 |
| deionized water | to 100% |

*Colorant is PURICOLOR BLUE ABL9 (FD&C Blue No. 1)

About 20 mL of each of the aqueous test formulations are placed in a borosilicate glass bottle. The glass bottles are exposed in an Atlas Ci-65 Xenon arc WeatherOmeter, AATCC Test Method 16. Color measurements are performed on a Hunter Ultrascan XE spectrophotometer. Delta L, a and b values are the difference between the initial values and the values at each interval. It is seen that the stabilizers of the present invention provide excellent color stability in personal care products.

### EXAMPLE 64

An aqueous based test formulation is prepared as follows:

| | |
|---|---|
| sodium laureth sulfate | 30% |
| (30%, TEXAPON NSO, Cognis) | |
| cocamidopropylbetaine | 10% |
| (30%, DEHYTON K, Cognis) | |
| colorant* | 0.001% |
| instant stabilizer | 0.05% |
| citric acid (10% aqueous solution) | to pH 6 |
| deionized water | to 100% |

*Colorant is PURICOLOR RED ARE33 (FD&C Red No. 33).

About 20 mL of each of the aqueous test formulations are placed in a borosilicate glass bottle. The glass bottles are exposed in an Atlas Ci-65 Xenon arc WeatherOmeter, AATCC Test Method 16, option E. Color measurements are performed on a Hunter Ultrascan XE spectrophotometer. Delta L, a and b values are the difference between the initial values and the values at each interval. It is seen that the stabilizers of the present invention provide excellent color stability in personal care products.

### EXAMPLE 65

An aqueous based test formulation is prepared as follows:

| | |
|---|---|
| sodium laureth sulfate | 30% |
| (30%, TEXAPON NSO, Cognis) | |
| cocamidopropylbetaine | 10% |
| (30%, DEHYTON K, Cognis) | |
| colorant* | 0.001% |
| instant stabilizer | 0.05% |
| citric acid (10% aqueous solution) | to pH 6 |
| deionized water | to 100% |

*Colorant is FD&C Red No. 40.

About 20 mL of each of the aqueous test formulations are placed in a borosilicate glass bottle. The glass bottles are exposed in an Atlas Ci-65 Xenon arc WeatherOmeter, AATCC Test Method 16. Color measurements are performed on a Hunter Ultrascan XE spectrophotometer. Delta L, a and b values are the difference between the initial values and the values at each interval. It is seen that the stabilizers of the present invention provide excellent color stability in personal care products.

### EXAMPLE 66

An aqueous based test formulation is prepared as follows:

| | |
|---|---|
| sodium laureth sulfate | 30% |
| (30%, TEXAPON NSO, Cognis) | |
| cocamidopropylbetaine | 10% |
| colorant* | 0.001% |
| instant stabilizer | 0.05% |
| citric acid (10% aqueous solution) | to pH 6 |
| deionized water | to 100% |

*Colorant is PURICOLOR BLUE ABL9 (FD&C Blue No. 1)

About 20 mL of each of the aqueous test formulations are placed in a borosilicate glass bottle. The glass bottles are also exposed to accelerated fluorescent lighting, Philips, 40 Watt, Daylight Deluxe (D65), full exposure to light. Color measurements are performed on a Hunter Ultrascan XE spectrophotometer. Delta L, a and b values are the difference between the initial values and the values at each interval. It is seen that the stabilizers of the present invention provide excellent color stability in personal care products.

### EXAMPLE 67

An aqueous based test formulation is prepared as follows:

| | |
|---|---|
| sodium laureth sulfate | 30% |
| (30%, TEXAPON NSO, Cognis) | |
| cocamidopropylbetaine | 10% |
| (30%, DEHYTON K, Cognis) | |
| colorant* | 0.001% |
| instant stabilizer | 0.05% |
| citric acid (10% aqueous solution) | to pH 6 |
| deionized water | to 100% |

*Colorant is PURICOLOR RED ARE33 (FD&C Red No. 33).

About 20 mL of each of the aqueous test formulations are placed in a borosilicate glass bottle. The glass bottles are also exposed to accelerated fluorescent lighting, Philips, 40 Watt, Daylight Deluxe (D65), full exposure to light. Color measurements are performed on a Hunter Ultrascan XE spectrophotometer. Delta L, a and b values are the difference between the initial values and the values at each interval. It is seen that the stabilizers of the present invention provide excellent color stability in personal care products.

### EXAMPLE 68

An aqueous based test formulation is prepared as follows:

| | |
|---|---|
| sodium laureth sulfate | 30% |
| (30%, TEXAPON NSO, Cognis) | |
| cocamidopropylbetaine | 10% |
| (30%, DEHYTON K, Cognis) | |
| colorant* | 0.001% |
| instant stabilizer | 0.05% |
| citric acid (10% aqueous solution) | to pH 6 |
| deionized water | to 100% |

*Colorant is FD&C Red No. 40.

About 20 mL of each of the aqueous test formulations are placed in a borosilicate glass bottle. The glass bottles are also exposed to accelerated fluorescent lighting, Philips, 40 Watt, Daylight Deluxe (D65), full exposure to light. Color measurements are performed on a Hunter Ultrascan XE spectrophotometer. Delta L, a and b values are the difference between the initial values and the values at each interval. It is seen that the stabilizers of the present invention provide excellent color stability in personal care products.

### EXAMPLE 69

The components of phase A are thoroughly mixed in a homogenizer for 10 min at 75-80° C. The water phase B, likewise heated to 75-80° C. beforehand, is slowly added and the mixture is homogenized for 1 min. The mixture is cooled, with stirring, to 40° C. and then phases C and E are added and the mixture is homogenized for 1 min. Subsequently, phase D is added and the mixture is homogenized for ½ min and cooled, with stirring, to room temperature.

| Phase | Ingredients | (w/w) % |
|---|---|---|
| A | passionflower oil | 8 |
| | glyceryl dioleate | 4 |
| | dicapryl ether | 4 |
| | isopropylisostearate | 4 |
| | instant stabilizer | 0.05 |
| B | water, demin. | ad. 100 |
| | EDTA | 0.1 |
| C | carbomer | 0.15 |
| D | sodium hydroxide | 10% |
| | | 0.20 |
| E | perfume; preservative | q.s. |

It is seen that the stabilizers of the present invention provide excellent color stability in personal care products.

### EXAMPLE 70

The components below are thoroughly mixed in the cited sequence at 50° C., a clear homogeneous solution being obtained. The UV absorber is, for example, 3-(2H-benzotriazol-2-yl)-4-hydroxy-5-(1-methylpropyl)-benzenesulfonic acid monosodium salt.

| Ingredients | (w/w) % |
|---|---|
| ethanol, 96% | 60 |
| d-limonene | 5 |
| cedrene | 1.5 |
| citronellol | 0.5 |
| savin | 0.5 |
| instant stabilizer | 0.08 |
| UV absorber | 0.1 |
| S,S-EDDS | 0.005 |
| colorant (D&C Yellow No. 5) | 0.02 |
| water | ad. 100 |

Excellent results are achieved for this example of a toilet water formulation.

### EXAMPLE 71

The hydroxypropyl cellulose is first predissolved in half of the alcohol (Vortex mixer) and is charged with the aminomethylpropanol. The other components—with the exception of the acrylate resin—are dissolved in alcohol and this solution is added, with stirring, to the hydroxypropyl cellulose. Subsequently, the acrylate resin is added and stirred until completely dissolved. The UV absorber used is, for example, benzophenone-4 is 5-benzoyl-4-hydroxy-2-methoxybenzenesulfonic acid, sodium salt.

| Ingredients | (w/w) % |
|---|---|
| alcohol, anhydrous | 96.21 |
| octylacrylamide/acrylate/ | 2.52 |
| butylaminoethylmethacrylate copolymer | |
| hydroxypropyl cellulose | 0.51 |
| aminomethylpropanol (95%) | 0.46 |
| instant stabilizer | 0.05 |
| UV absorber | 0.05 |
| perfume oil | 0.20 |

Excellent results are achieved for this example of a hair styling spray formulation.

### EXAMPLE 72

The components listed below are mixed, with stirring, at room temperature until they are completely dissolved. The pH is 6.5. The UV absorber is, for example, 2-(2-hydroxy-3-dodecyl-5-methylphenyl)-2H-benzotriazole.

| Ingredients | (w/w) % |
|---|---|
| sodium myreth sulfate | 50.00 |
| TEA abietoyl collagen hydrolysate | 3.50 |
| laureth-3 | 3.00 |
| colorant (D&C Red No. 33) | 0.20 |
| instant stabilizer | 0.05 |
| UV absorber | 0.15 |
| phosphonomethylchitosan, sodium salt | 0.01 |
| perfume oil | 0.10 |
| water | ad. 100 |

Excellent results are achieved for this example of a shampoo composition for oily hair.

### EXAMPLE 73

The stabilizer is predissolved in the terpene. The components are then stirred in the cited sequence at about 65° C. until homogeneous. The mixture is then cooled to room temperature.

| Ingredients | (w/w) % |
|---|---|
| synthetic soap (ZETESAP 813) | 7.85 |
| glycerol | 6.00 |
| anionic surfactant (LUMOROL 4192; | 22.00 |
| MULSIFAN RT 13) | |
| VASELINE | 11.00 |
| PARAFFIN 52/54 | 20.00 |
| talcum | 2.00 |
| orange terpene | 4.00 |
| instant stabilizer | 0.02 |
| water | 27.13 |

Excellent results are achieved for this example of a leather dressing and cleaning agent composition.

### EXAMPLE 74

The components listed below are dissolved in the cited sequence until a clear homogeneous mixture is obtained.

| Ingredients | (w/w) % |
|---|---|
| anionic/amphoteric surfactants (Lumorol RK) | 0.7 |
| butyl glycol | 5.0 |
| isopropanol | 20.0 |
| d-limonene | 4.00 |
| instant stabilizer | 0.02 |
| water, demin. | ad. 100 |

Excellent results are achieved for this example of a glass detergent formulation.

### EXAMPLE 75

The instant stabilizers are each deposited (from water) on a dyed cotton fabric at 0.05, 0.1, 0.2, 0.5 and 1.0 percent by weight, based on the weight of the cotton. The dyed fabrics contain the following dyes at 0.05, 0.1, 0.2 and 0.5 percent by weight based on cotton. This results in 60 separate formulations for each dye listed:

Scarlet HE-3G

Crimson HE-XL

Yellow HE-6G

Red HE-XL

Blue HE-XL

Turquoise H-A

Navy HE-XL

Remazol

Red RB

Brilliant Red RBS

Orange FR

Navy CG

Turquoise G

Black B

The cotton fabrics are subjected to light exposure in an Atlas Ci-65 Xenon arc WetherOmeter or to accelerated fluorescent lighting. The present stabilizers provide outstanding color protection to the dyed fabrics. This experiment simulates dye protection achievable through deposition of the present stabilizers via treatment with, for example, stabilizer-containing laundry detergent or fabric conditioner.

### EXAMPLE 76

The instant stabilizers and UV absorbers, for example 3-(2H-benzotriazol-2-yl)-4-hydroxy-5-(1-methylpropyl)-benzenesulfonic acid monosodium salt, are each deposited (from water) on a dyed cotton fabric at 0.05, 0.1, 0.2, 0.5 and 1.0 percent by weight, based on the weight of the cotton. The dyed fabrics contain the following dyes at 0.05, 0.1, 0.2 and 0.5 percent by weight based on cotton. This results in 60 separate formulations for each dye listed:

Scarlet HE-3G

Crimson HE-XL

Yellow HE-6G

Red HE-XL

Blue HE-XL

Turquoise H-A

Navy HE-XL

Remazol

Red RB

Brilliant Red RBS

Orange FR

Navy CG

Turquoise G

Black B

The cotton fabrics are subjected to light exposure in an Atlas Ci-65 Xenon arc WetherOmeter or to accelerated fluorescent lighting. The present stabilizers provide outstanding color protection to the dyed fabrics. This experiment simulates dye protection achievable through deposition of the present stabilizers via treatment with for example stabilizer-containing laundry detergent or fabric conditioner.

### EXAMPLE 77

Molded test specimens are prepared by injection molding thermoplastic olefin (TPO) pellets containing pigments, a phosphite, a phenolic antioxidant or hydroxylamine, a metal stearate, ultraviolet light absorbers or a hindered amine stabilizer or a mixture of UV absorber and hindered amine stabilizer.

Pigmented TPO pellets are prepared from pure pigment or pigment concentrate, coadditives and commercially available TPO by mixing the components in a Superior/MPM 1″ single screw extruder with a general all-purpose screw (24:1 L/D) at 400° F. (200° C.), cooled in a water bath and pelletized. The resulting pellets are molded into 60 mil (0.006 inch), 2″×2″ plaques at about 375° F. (190° C.) on a BOY 30M Injection Molding Machine.

Pigmented TPO formulation composed of polypropylene blended with a rubber modifier where the rubber modifier is an in-situ reacted copolymer or blended product containing copolymers of propylene and ethylene with or without a ternary component such as ethylidene norbornene are stabilized with a base stabilization system consisting of an N,N-dialkylhydroxylamine or a hindered phenolic antioxidant with or without an organophosphorus compound.

All additive and pigment concentrations in the final formulation are expressed as weight percent based on the resin.

Formulation contained thermoplastic olefin pellets and one or more of the following components:

0.0 to 2.0% pigment,

0.0 to 50.0% talc,

0.0 to 0.1% phosphite,

0.0 to 1.25% phenolic antioxidant,

0.0 to 0.1% hydroxylamine

0.05 to 0.10 calcium stearate,

0.0 to 1.25% UV absorber

0.0 to 1.25% hindered amine stabilizer.

The components are dry-blended in a tumble dryer prior to extrusion and molding.

Test plaques are mounted in metal frames and exposed in an Atlas Ci65 Xenon Arc Weather-Ometer at 70° C. black panel temperature, 0.55 W/m² at 340 nanometers and 50% relative humidity with intermittent light/dark cycles and water spray (Society of Automotive Engineers—SAE J 1960 Test Procedure). Specimens are tested at approximately 625 kilojoule intervals by performing color measurements on an Applied Color Systems spectrophotometer by reflectance mode according to ASTM D 2244-79. Data collected include delta E, L*, a* and b* values. Gloss measurements are conducted on a BYK-Gardner Haze/Gloss Meter at 60° according to ASTM D 523.

### UV Exposure Testing

Test specimens exposed to UV radiation exhibit exceptional resistance to photodegradation when stabilized with light stabilizer systems comprising a combination of 2-(2-hydroxy-3,5-di-tert-amylphenyl)-2H-benzotriazole, an instant stabilizer and N,N′,N″,N″′-tetrakis[4,6-bis(butyl-(1,2,2,6,6-pentamethylpiperidin-4-yl)amino)-s-triazin-2-yl]-1,10-diamino-4,7-diazadecane. The control sample consists of a stabilizer formulation commonly used in the industry to impart UV stability. All of the samples contain a pigment, Pigment Red 177, and talc.

The test plaques described earlier contain the following (all concentrations are weight percent based on resin):

Polymer substrate is commercially available polyolefin blend POLYTROPE® TPP 518-01 supplied by A. Schulman Inc. Akron, Ohio)

Color package is 0.025% Red 3B -Pigment Red 177, C.I. #65300.

Each plaque contains:

0.2% 2-(3,5-di-t-amyl-2-hydroxyphenyl)-2H-benzotriazole;

0.1% calcium stearate; and

15% talc.

The Control plaques additionally contain

0.1% 50:50 blend of neopentanetetrayl tetrakis(4-hydroxy-3,5-di-tert-butylhydrocinnamate) and [tris-(2,4-di-tert-butylphenyl) phosphite;

0.2% [bis(2,2,6,6-tetramethylpiperidin-4-yl) sebacate];

0.2% [polycondensation product of 4,4′-hexamethylene-bis(amino-2,2,6,6-tetramethylpiperidine) and 2,4-dichloro-6-tert-octylamino-s-triazine].

The test plaques containing the instant stabilizers additionally each contain 0.05% N,N,-dialkylhydroxylamine;

The instant stabilizers demonstrate greatly improved gloss retention compared to the less effective control system. Resistance to color change upon UV exposure is also enhanced.

Polymer blends containing an unsaturated ternary component, such as EPDM blends, are especially benefited with the more efficient instant light stabilizer systems described above.

### EXAMPLE 78

Molded test specimens are prepared by injection molding thermoplastic olefin (TPO) pellets containing the instant compounds, pigments and other coadditives as described in Example 77.

The light stable formulations are painted with one-pack paint systems and tested for TPO/paint interactions. Before painting, the test specimens are first washed in accordance with GM998-4801 and dried for 15 minutes at 200° F. (94° C.). Adhesion promoter is applied to the dry film thickness of 0.2-0.4 mils. The samples are dried for five minutes before a 1K basecoat is applied to a film thickness of 1.2-1.4 mils. The painted panels are dried for three minutes, a clearcoat is then applied to a dry film thickness of 1.2-1.5 mils followed by ten minutes flash drying and a 30 minute oven bake at 250° F. (121° C.).

Paint adhesion is measured by Aggressive Adhesion Testing (proprietary test procedure conducted at Technical Finishing, Inc.) and Taber Scuff. Painted panels which retain greater than 80% of the paint finish are considered acceptable. After Aggressive Adhesion Testing, samples with less than 5% paint loss are deemed acceptable.

The instant compounds provide very low levels of paint loss when analyzed by the testing protocols listed above.

### EXAMPLE 79

Molded test specimens are prepared by injection molding polypropylene pellets containing pigments, a phosphite, a phenolic antioxidant or hydroxylamine, a metal stearate, ultraviolet light absorbers or a hindered amine stabilizers or a mixture of UV absorbers and hindered amine stabilizers.

Pigmented polypropylene pellets are prepared from pure pigment or pigment concentrates, stabilizers, co-additives and commercially available polypropylene by mixing the components in a Superior/MPM 1″ single screw extruder with a general all-purpose screw (24:1 L/D) at 475° F. (250° C.), cooled in a water bath and pelletized. The resulting pellets are molded into 60 mil (0.06 inch thick) 2″×2″ plaques at about 475° F. (250° C.) on a BOY 30M Injection Molding Machine.

Pigmented polypropylene formulations composed of polypropylene homopolymer or polypropylene copolymer are stabilized with a base stabilization system consisting of an N,N-dialkylhydroxylamine or a hindered phenolic antioxidant with or without an organophosphorous compound.

All additive and pigment concentrations in the final formulations are expressed as weight percent based on the resin.

Formulations contained polypropylene pellets and one or more of the following components;

0.0%-2.0% pigment,

0.0%-50.0% talc,

0.0%-50.0% calcium carbonate,

0.0%-0.1% phosphite,

0.0%-1.25% phenolic antioxidant,

0.0%-0.1% hydroxylamine,

0.05%-0.10% calcium stearate,

0.0%-1.25% UV absorber,

0.0%-1.25% hindered amine stabilizer.

The components are dry blended in a tumble dryer prior to extrusion and molding.

Test plaques are mounted in metal frames and exposed in an Atlas Ci65 Xenon Arc Weather-o-meter at 70° C. black panel temperature, 0.55 W/m² at 340 nanometers and 50% relative humidity with intermittent light/dark cycles and water spray (Society of Automotive Engineers—SAE J 1960 Test Procedure). Specimens are tested at approximately 625 kilojoule intervals by performing color measurements on an Applied Color Systems spectrophotometer by reflectance mode according to ASTM D 2244-79. Data collected included delta E, L*, a* and b* values. Gloss measurements are conducted on a BYK-GARDNER Haze/Gloss Meter at 60° according to ASTM D523.

### UV Exposure Testing

Test specimens exposed to UV radiation exhibit exceptional resistance to photodegradation when stabilized with light stabilizer systems comprised of a combination of 2-(3,5-di-t-amyl-2-hydroxyphenyl)-2H-benzotriazole, an instant stabilizer, and an oligomeric hindered amine. The oligomeric hindered amine is oligomer of N-{[2-(N-2,2,6,6-tetramethylpiperidin-4-yl)butylamino]-s-triazin-4-yl}-N,N′-bis(2,2,6,6-tetramethylpiperidin-4-yl)-1,6-hexanediamine terminated with 2,4-bis(dibutylamino)-s-triazin-6-yl. The Control sample consists of a stabilizer formulation commonly used in the industry to impart UV stability. All of the samples contain Pigment Red 177.

All formulations are base stabilized with 0.05% dialklyhydroxylamine in the final resin formulation.

Polymer substrate is a commercially available polypropylene homopolymer—Profax 6501 (commercial supplier Montell Polyolefins).

Color package is 0.25% Red 3B—Pigment Red 177, C.I. # 65300 in the final resin formulation.

Each formulation contains a hydroxyphenyl benzotriazole UV absorber—2-(2-hydroxy-3,5-di-tert-amylphenyl)-2H-benzotriazole.

Each formulation contains 0.1% calcium stearate.

Samples are 60 mil thick 2″×2″ injection molded plaques.

UV exposures conducted under SAE J 1960—Exterior Automotive conditions.

All additive and pigment concentrations in the final formulations are expressed as weight percent on the resin.

The light stabilized formulations show much greater resistance to photodegradation than unstabilized specimens which fail quickly under the UV exposure conditions outlined above.

### EXAMPLE 80

Fiber samples are prepared by extruding fiber-grade polypropylene with the instant compounds, coadditives and pigments. Typical formulations contain the instant compounds at levels from 0.05 to 2.0%, a metal stearate such as calcium stearate at 0.05 to 0.5%, pigments from 0 to 5%, UV absorbers at levels of 0.05 to 2.0%, phosphites at 0 to 0.1%, phenolic antioxidants at 0 to 1.25%, N,N-dialkylhydroxylamines at 0 to 0.1% and optionally other hindered amines at levels of 0 to 2.0%. All additive and pigment concentrations in the final formulations are given as weight percent based on the resin.

Pigment concentrates are prepared from pure pigment and polypropylene (PROFAX®, Hercules) by mixing the two components in a high shear mixer in a ratio of 25% pigment and 75% resin, pressing the resulting resin/pigment mixture on a Wabash Compression molder (Model # 30-1515-4T3) into a thin sheet and dividing the sheet into fine chips for dispersion in polypropylene at reduced concentrations. Alternatively, pigment concentrates are obtained as pigment dispersions in a suitable carrier resin for subsequent blending in fiber at reduced concentrations.

Formulations containing polypropylene, 0.05-0.1% phosphite, 0-1.25% phenolic antioxidant, 0-0.1% dialkylhydroxylamine, 0.05-0.1% calcium stearate, 0-1.25% UV absorber, 0-1.25% hindered amine are dry blended in a tumble dryer, extruded on a Superior/MPM 1″ single screw extruder with a general all-purpose screw (24:1 L/D) at 475° F. (246° C.), cooled in a water bath and pelletized. The resulting pellets are spun into fiber at about 475° F. (246° C.) on a HILLS Research Fiber Extruder (Model # REM-3P-24) fitted with a 41 hole, delta configuration spinneret. The spun tow is stretched at a draw ratio of 3.2:1 producing a final denier of 615/41.

Fiber samples are knitted into socks on a Lawson-HemphillFiber Analysis Knitter, cut into appropriate lengths and exposed in an Atlas Ci65 Xenon Arc Weather-O-meter at 89° C. black panel temperature, 0.55 W/m² at 340 nanometers and 50% relative humidity (Society of Automotive Engineers—SAE J 1885 Test Procedure).

Fiber samples are tested by performing color measurements on an Applied Color Systems spectrophotometer by reflectance mode according to ASTM D 2244-79. Identical, but separate, fiber samples are examined for catastrophic failure and the time to failure is recorded.

The samples containing the instant compounds exhibit good stabilization performance against the deleterious effects of UV light.

### EXAMPLE 81

Other socks of propylene fiber as prepared in Example 80 are exposed in a Blue M forced draft oven at 120° C. Failure is determined by the criterion set forth in Example 80. The longer it takes for the catastrophic failure to occur, the more effective is the stabilizing system.

The socks containing the instant compounds exhibit good thermal stabilization efficacy.

### EXAMPLE 82

Film grade polyethylene is dry blended with approximately 10.0% by weight of the test additives and then melt compounded at 200° C. into “Masterbatch” pellets. The fully formulated “Masterbatch” pellets are dry blended with polyethylene resin to get the desired final stabilizer concentrations. Typical formulations contain the instant compounds at levels from 0.05% to 2.0%, a metal stearate such as calcium stearate at 0.05% to 0.5%, a phosphite at 0% to 0.1%, a phenolic antioxidant at 0% to 1.25%, an N,N-dialkylhydroxylamine at 0% to 0.1% and optionally a hindered amine at 0% to 2.0%. This stabilized fully formulated resin is then blown at 200° C. into a 150 micron thick film on a DOLCI film line.

The blown films are exposed in an Atlas Xenon-Arc WeatherOmeter according to ASTM G26 at 63° C. bpt, 0.35 W/m² at 340 nm with no spray cycle. Films are tested periodically for any change in elongation using an Instron 112 tensile tester. Failure in this test is determined by observation of the loss of % elongation in the film. The longer it takes for this loss to occur, the more effective is the stabilizer system.

The films containing the instant compound mixture show good light stabilizing efficacy.

### EXAMPLE 83

Film grade polyethylene is dry blended with 10% loading of the test additives, as described in Example 82, and then melt compounded at 200° C. into fully formulated master batch pellets. The master batch pellets are dry blended with the polyethylene resin to get the final stabilizer concentration. The fully formulated resin is then blown at 200° C. into a 150 micron thick film using a DOLCI film line.

The resulting films are exposed on a greenhouse on galvanized iron backing. Treatment includes applications of pesticides on a regular basis (i.e. sodium N-methyldithiocarbamate, VAPAM [symbol] every six months and SESMETRIN [symbol] every month). Performance is measured by monitoring the percent residual elongation. Failure is defined as the time to a 50% loss of original elongation.

The films containing the instant compounds show good resistance to pesticides.

### EXAMPLE 84

Master batch pellets prepared as described in Example 82 are dry blended into polyethylene resin to get the final stabilizer concentration. The fully formulated resin is then blown at 200° C. into a 25 micron thick film using a DOLCI film line.

The resulting films are exposed on a soil to simulate agricultural mulch film conditions. Treatment includes exposure to methyl bromide fumigant for three days at 60 g/m³. Performance is measured by monitoring the time to physical embrittlement.

The films containing the instant compounds show good resistance to fumigants.

### EXAMPLE 85

Greenhouse film samples are prepared as described in Example 82, but in addition to the instant compounds also contain a metal stearate or a metal oxide. Typical formulations contain from 0.05 to 2% by weight of the instant hindered amines, 0.05 to 0.5% of a metal stearate such as calcium oxide, and 0.05 to 0.5% of a metal oxide such as zinc oxide or magnesium oxide.

Effectiveness is monitored as described in Example 83. The films containing the instant compounds exhibit good light stability.

### EXAMPLE 86

Polypropylene fiber is prepared as described in Example 80. In addition to the instant compounds, selected halogenated flame retardants are also included in the formulation. The flame retardants are tris(3-bromo-2,2-bis(bromomethyl)propyl)phosphate, decabromodiphenyl oxide, ethylene bis-(tetrabromophthalimide), or ethylene bis-(dibromo-norbornanedicarboximide).

Using the criterion for light stabilization described in Example 80, the socks knitted from the polypropylene fiber containing the instant compounds exhibit good light stability.

### EXAMPLE 87

Molding grade polypropylene is dry blended with test additives and then melt compounded into pellets. In addition to the instant compounds, selected flame retardants are also included. The flame retardants are tris(3-bromo-2,2-bis(bromomethyl)propyl)phosphate, decabromodiphenyl oxide, ethylene bis-(tetrabromophthalimide), or ethylene bis-(dibromo-norbornanedicarboximide). The pelletized fully formulated resin is then injection molded into test specimens using a Boy 50M laboratory model injection molder.

Test plaques are mounted in metal frames and exposed in an Atlas Ci65 Xenon Arc Weather-Ometer with intermittent light/dark cycles and water spray following the ASTM G26 test procedure. Specimens are tested at periodic intervals for changes in tensile properties. Failure in this test is determined by the observation of the loss of tensile properties. The longer it takes for the loss in properties to occur, the more effective is the stabilizer system.

The test samples containing the instant compounds exhibit good light stabilization properties.

### EXAMPLE 88

Molded test specimens are prepared by injection molding thermoplastic olefin (TPO) pellets as described in Example 77. In addition to the instant compounds, selected flame retardants are also included in the test specimens. The flame retardants are tris(3-bromo-2,2 bis(bromomethyl)propyl) phosphate, decabromodiphenyl oxide, ethylene bis-(tetrabromo-phthalimide), or ethylene bis-(dibromo-norbornanedicarboximide).

The samples including the instant hindered amines exhibit good light stabilizing activity.

### EXAMPLE 89

Film grade polyethylene is compounded and blown into film at 200° C. as described in Example 83 using a DOLCI film line. In addition to the instant compounds, selected flame retardants are included in the formulation. The flame retardants are tris(3-bromo-2,2-bis(bromomethyl)propyl) phosphate, decabromodiphenyl oxide, ethylene bis-(tetrabromophthalimide), or ethylene bis-(dibromo-norbornanedicarboximide).

When tested for light stabilizing activity as described in Example 83, the films containing the instant compounds exhibit good stabilization.

### EXAMPLE 90

Molded test specimens are prepared by injection molding thermoplastic olefin (TPO) pellets containing the instant compounds, pigments and other coadditives as described in Example 78.

The test specimens are painted with one-pack paint systems and tested for TPO/paint interactions. Before painting, the test specimens are first wiped with isopropanol and air blasted to remove any dust. After a five minute flash, these specimens are coated with the adhesion promoter, then the base coat, and then optionally a clear coat. Typical film thickness of these various coatings are 0.1-0.3 mils for the adhesion promoter, 0.6-0.8 mils for the base coat, and 1.2-1.5 mils for the clear coat. After painting, the specimens are cured in an over at 120° C. for 30 minutes.

Samples are tested to evaluate the TPO/paint interactions as follows: In the initial adhesion test, a clear cellophane adhesive tape is used to pull on a 3 mm cross hatched paint surface or; in the humidity test, the painted plaques are exposed for 240 hours at 38° C. in an atmosphere having 98% relative humidity. The blister rating is tested by visual observation according to ASTM D 714.

The samples containing the instant compounds exhibit good TPO/paint interaction properties as determined by the criteria above.

### EXAMPLE 91

Resin materials of the general class known as thermoplastic elastomers, examples of which include, copolymers of styrene with butadiene or isoprene and/or ethylene-cobutylene such as SBS, SEBS and SIS, are dry blended with the instant compounds and melt compounded into pellets. Typical formulations contain the instant compounds at levels from 0.05% to 2.0%, a metal stearate such as calcium stearate at 0.05% to 0.5%, pigments from 0% to 5%, UV absorbers at levels of 0.05% to 2.0%, phosphites at 0.0%-0.1%, phenolic antioxidants at 0.0%-1.25%, N,N-dialkylhydroxylamine at 0.0%-0.1%, and optionally other hindered amine stabilizers at levels of 0.0% to 2.0%.

The pelletized fully formulated resin is then processed into a useful article such as blown or cast extrusion into film; injection molded into a molded article; thermoformed into molded articles; extruded into wire and cable housing; or rotational molded into hollow articles.

The materials containing the instant compounds exhibit stability against deleterious effects of UV light and thermal exposure.

### EXAMPLE 92

Articles prepared according to Example 91 which additionally contain selected organic pigments as well as the instant compounds also exhibit stability against the deleterious effects of actinic light and thermal exposure.

### EXAMPLE 93

Articles prepared according to Example 91 which additionally contain a hindered phenolic antioxidant selected from the group consisting of neopentanetetrayl tetrakis(3,5-di-tert-butyl-4-hydroxyhydrocinnamate, octadecyl 3,5-di-tert-butyl-4-hydroxyhydrocinnamate, 1,3,5-trimethyl-2,4,6-tris(3,5,-di-tert-butyl-4-hydroxybenzyl)benzene, 1,2-bis(3,5-di-tert-butyl-4-hydroxyhydrocinnamoyl)hydrazine, calcium [bis(monoethyl 3,5-ditert-butyl-4-hydroxybenzyl)phosphonate], 1,3,5-tris(3,5-di-tert-butyl-4-hydroxybenzyl) isocyanurate and 1,3,5-tris(3-hydroxy-4-tert-butyl-2,6-dimethylbenzyl) isocyanurate, as well as the instant compounds also exhibit stability against the deleterious effects of actinic light and thermal exposure.

### EXAMPLE 94

Articles prepared according to Example 91 which additionally contain an organophosphorus stabilizer selected from the group consisting of tris(2,4-di-tert-butylphenyl) phosphite, bis(2,4-di-tert-butyl-6-methylphenyl) ethyl phosphite, 2,2′,2″-nitrilo[triethyl-tris-(3,3′,5,5′-tetra-tert-butyl-1,1′-biphenyl-2,2′-diyl) phosphite], tetrakis(2,4-di-butylphenyl) 4,4′-biphenylenediphosphonite, tris(nonylphenyl) phosphite, bis(2,4-di-tert-butylphenyl) pentaerythrityl diphosphite, 2,2′-ethylidenebis(2,4-di-tert-butylphenyl) fluorophosphite and 2-butyl-2-ethylpropan-1,3-diyl 2,4,6-tri-tert-butylphenyl phosphite as well as the instant compounds also exhibit stability against the deleterious effects of actinic light and thermal exposure.

### EXAMPLE 95

Articles prepared according to Example 91 which additionally contain a benzofuranone stabilizer which is 5,7-di-tert-butyl-3-(3,4-dimethylphenyl)-2H-benzofuran-2-one, as well as the instant compounds also exhibit stability against the deleterious effects of UV light and thermal exposure.

### EXAMPLE 96

Articles prepared according to Example 91 which additionally contain a dialkylhydroxylamine stabilizer which is N,N-dialkylhydroxylamine made by the direct oxidation of N,N-di(hydrogenated tallow)amine as well as the instant compounds also exhibit stability against the deleterious effects of actinic light and thermal exposure.

### EXAMPLE 97

Articles prepared according to Example 91 which additionally contain other hindered amine stabilizers selected from the group consisting of bis(2,2,6,6-tetramethylpiperidin-4-yl) sebacate, the polycondensation product of 1-(2-hydroxyethyl)-2,2,6,6-tetramethyl-4-hydroxypiperidine and succinic acid, N.N′,N″,N″′-tetrakis[4,6-bis(butyl-1,2,2,6,6-pentamethylpiperidin-4-yl)amino-s-triazin-2-yl]-1,10-diamino-4,7-diazadecane, the polycondensation product of 4,4′-hexamethylenebis(amino-2,2,6,6-tetramethylpiperidine) and 2,4-dichloro-6-tert-octylamino-s-triazine, the polycondensation product of 4,4′-hexamethylenebis(amino-2,2,6,6-tetramethylpiperidine) and 2,4-dichloro-6-morpholino-s-triazine, 2,2,6,6-tetramethylpiperidin-4-yl octadecanoate, 3-dodecyl-1-(1-acetyl-2,2,6,6-tetramethylpiperidin-4-yl)-pyrrolidin-2,5-dione, 1,3,5-tris{N-cyclohexyl-N-[2-(2,2,6,6-tetramethylpiperazin-3-on-4-yl)ethyl]amino}-s-triazine, poly[methyl 3-(2,2,6,6-tetramethylpiperidin-4-yloxy)propyl]siloxane, the polycondensation product of 2,4-dichloro-6-(2,2,6,6-tetramethylpiperidin-4-yl)butylamino)-s-triazine and 2,2′ethylene-bis{[2,4-(2,2,6,6-tetramethylpiperidin-4-yl)butylamino-s-triazin-6-yl]amino-trimethyleneamino} as well as the instant compounds also exhibit stability against the deleterious effects of actinic light and thermal exposure.

### EXAMPLE 98

Articles prepared according to Example 91 which additionally contain other N-hydrocarbyloxy substituted hindered amines selected from the group consisting of bis(1-octyloxy-2,2,6,6-tetramethylpiperidin-4-yl) sebacate, bis(1-octyloxy-2,2,6,6-tetramethylpiperidin-4-yl) adipate, bis(1-cyclohexyloxy-2,2,6,6-tetramethylpiperidin-4-yl) adipate, bis(1-cyclohexyloxy-2,2,6,6-tetramethylpiperidin-4-yl) sebacate, and 1-cyclohexyloxy-2,2,6,6-tetramethylpiperidin-4-yl octadecanoate as well as the instant compounds also exhibit stability against the deleterious effects of actinic light and thermal exposure.

### EXAMPLE 99

Articles prepared according to Example 91 which additionally contain a o-hydroxyphenyl-2H-benzotriazole, a hydroxyphenyl benzophenone or a o-hydroxyphenyl-s-triazine UV absorber selected from the group consisting of 2-(2-hydroxy-3,5-di-α-cumylphenyl)-2H-benzotriazole, 2-(2-hydroxy-5-methylphenyl)-2H-benzotriazole, 5-chloro-2-(2-hydroxy-3,5-di-tert-butylphenyl)-2H-benzotriazole, 2-(2-hydroxy-3,5-di-tert-amylphenyl)-2H-benzotriazole, 2-(2-hydroxy-3-alphacumyl-5-tert-octylphenyl)-2H-benzotriazole, 2,4-di-tert-butylphenyl 3,5-di-tert-butyl4-hydroxybenzoate, 2-hydroxy-4-n-octyloxybenzophenone and 2,4-bis(2,4-dimethyphenyl)-6-(2-hydroxy-4-octyloxyphenyl)-s-triazine as well as the instant compounds also exhibit stability against the deleterious effects of UV light and thermal exposure.

### EXAMPLE 100

### Polyester Urethane Coating

The hindered amine test stabilizers are incorporated into a two-component polyester urethane coating based on a commercially available polyester polyol (DESMOPHEN® 670-80) and commercially available isocyanurate (DESMODUR® N-3390) at a level of 2% by weight based on total resin solids. The coating system is catalyzed with 0.015% dibutyl tin dilaurate based on total resin solids.

Each coating formulation is applied by draw down onto transparent glass slides approximately 4″×6″ to a film thickness of about 2 mils (0.002″).

The instant compounds provide excellent solubility and compatibility in a polyester urethane coating.

### EXAMPLE 101

Formulation Reference: NeoResins, Inc., Formulation WB-2010, Technical Brochure, August, 2000.

The following components are mixed together under good agitation:

| | |
|---|---|
| Butyl cellusolve | 5.68 parts |
| Carbitol | 4.26 parts |
| Triton X-100 | 0.25 parts |
| Water | 1.66 parts |
| Instant compound | 0.58 parts |

This solution is added to a 4 ounce clear glass jar containing 72.85 parts of NeoPac R-9699 under vigorous agitation and agitated for 10 minutes. Optionally, a defoamer, like Dehydran 1620 (Henkel), and a flash rusting agent, like Heiscore XAB (Cas Chem), are added. The lid is placed securely on the jar. The solidified coatings are visually observed for clarity after solidification. The development of opacity or haziness is indicative of an incompatibility between the hindered amine stabilizer and the formulated coating.

Solidified coating in jar

| Sample* | 0 days | 1 day | 19 days |
|---|---|---|---|
| A | clear | clear | clear |
| B | hazy | hazy | hazy |
| C | clear | clear | clear |
| D | clear | clear | clear |
| E | clear | clear | clear |
| F | clear | clear | clear |
| G | clear | clear | clear |
| H | clear | clear | clear |
| I | clear | clear | clear |
| J | clear | clear | clear |
| K | clear | clear | clear |
| L | clear | clear | clear |
| M | clear | clear | clear |
| N | clear | clear | clear |

*A is unstabilized.
B contains 2% by weight of bis(1-octyloxy-2,2,6,6-tetramethylpiperidin-4-yl) sebacate.
C contains 2% by weight of Instant Example 17.
D contains 2% by weight of Instant Example 7.
E contains 2% by weight of Instant Example 12.
F contains 2% by weight of Instant Example 8.
G contains 2% by weight of instant Example 57.
H contains 2% by weight of Instant Example 59.
I contains 2% by weight of Instant Example 53.
J contains 2% by weight of Instant Example 62.
K contains 2% by weight of Instant Example 111.
L contains 2% by weight of Instant Example 107.
M contains 2% by weight of Instant Example 105.
N contains 2% by weight of Instant Example 109.

These data show that the instant compounds provide excellent solubility and compatibility in a high solids water borne urethane/acrylic copolymer coating system.

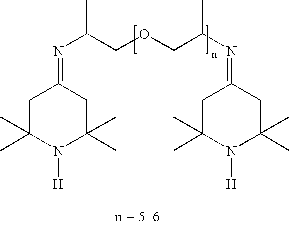

2,2,6,6-Tetramethylpiperid-4-one (35 g, 0.2 mole) and Jeffamine D-400 (38.2 g, 0.095 mole, Huntsman) are added to cyclehexane (300 mL). The solution is heated to reflux and refluxed for four hours while collecting the water by-product in a Dean-Stark trap. Xylene (300 mL) is added and reflux is continued for four more hours. The solvent is removed by vacuum distillation and the resulting product is dried to constant weight. The title compound is received (69.2 g) as a viscous amber liquid whose structure is consistent with HNMR.

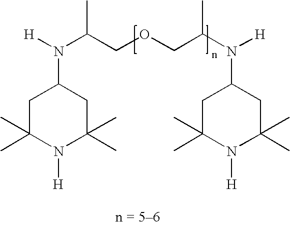

Example 102 (30 g, 0.044 mole) is dissolved in ethanol (250 mL). Sodium borohydride (1.7 g, 0.044 mole) is added to the solution in one portion with agitation. The resulting solution is stirred overnight at ambient temperature. The solvent is removed by vacuum distillation and the residue is redissolved in ethyl acetate. The solution is washed repeatedly with water. The organic layer is then dried, the solvent removed by vacuum distillation, and the residue is dried to constant weight. The title compound is received (28 g) as a light yellow oil whose structure is consistent with HNMR.

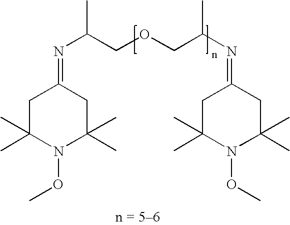

1-Methoxy-2,2,6,6-tetramethylpiperid-4-one (20 g, 0.108 mole) and Jeffamine D-400 (22 g, 0.049 mole, Huntsman) are added to toluene (300 mL). The solution is heated to reflux and refluxed for four hours while collecting the water by-product in a Dean-Stark trap. The solvent is removed by vacuum distillation and the resulting product is dried to constant weight. The title compound is received (41 g) as a light yellow liquid whose structure is consistent with HNMR.

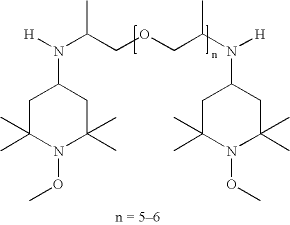

Example 104 (30 g, 0.04 mole) is dissolved in ethanol (300 mL). Sodium borohydride (1.5 g, 0.04 mole) is added to the solution in one portion with agitation. The resulting solution is stirred overnight at ambient temperature. The solvent is removed by vacuum distillation and the residue is redissolved in ethyl acetate. The solution is washed repeatedly with water. The organic layer is then dried, the solvent removed by vacuum distillation, and the residue is dried to constant weight. The title compound is received (26 g) as a light yellow oil whose structure is consistent with HNMR.

This is an example of a compound of formula (7) where R₅ is a homooligomer of propylene oxide.

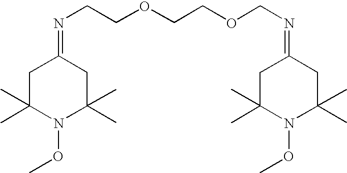

Following the procedure described in Example 104, 1-methoxy-2,2,6,6-tetramethylpiperid-4-one (25 g, 0.135 mole) and Jeffamine XTJ-504 (9.9 g, 0.067 mole, Huntsman) are reacted together. The title compound is received (32.4 g) as a light yellow oil whose structure is consistent with HNMR.

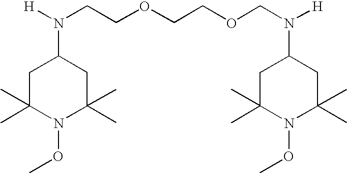

Following the synthetic procedure described in Example 105, Example 106 (32.4 g) is reduced to yield the title compound (26 g) as a light yellow oil whose structure is consistent with HNMR.

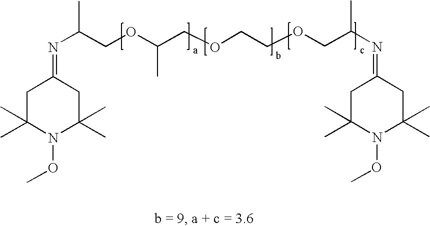

Following the procedure described in Example 104, 1-methoxy-2,2,6,6-tetramethylpiperid-4-one (6 g, 0.0324 mole) and Jeffamine XTJ-500 (10 g, 0.0166 mole, Huntsman) are reacted together. The title compound is received (15.4 g) as a light yellow oil whose structure is consistent with HNMR.

### EXAMPLE 109

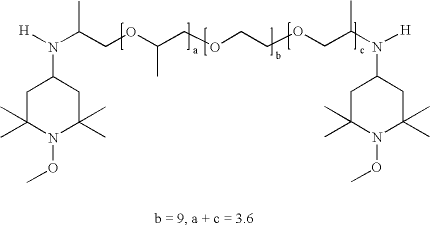

Following the synthetic procedure described in Example 105, Example 108 (15.4 g) is reduced to yield the title compound (14 g) as a light yellow oil whose structure is consistent with HNMR.

This compound is an example of a compound of formula (7) where R₅ is a co-oligomer of ethylene oxide and propylene oxide.

### EXAMPLE 110

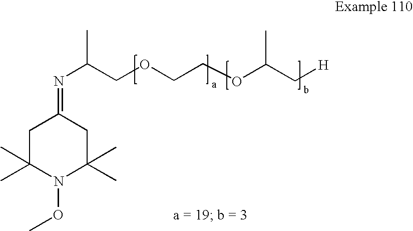

Following the procedure described in Example 104, 1-methoxy-2,2,6,6-tetramethylpiperid-4-one (25 g, 0.135 mole) and Jeffamine XTJ-506 (128 g, 0.128 mole, Huntsman) are reacted together. The title compound is received (150.5 g) as a light yellow oil whose structure is consistent with HNMR.

### EXAMPLE 111

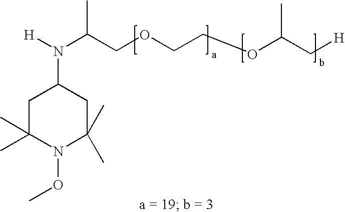

Following the synthetic procedure described in Example 105, Example 110 (150.5 g) is reduced to yield the title compound (44.1 g) as a light yellow oil whose structure is consistent with HNMR.

### EXAMPLE 112

Formulation Reference: NeoResins, Inc., Formulation WB-2065, Technical Brochure.

The following components are mixed together under good agitation:

| | |
|---|---|
| Water | 31.2 parts |
| Aqua Ammonia (26%) | 1.3 parts |

This solution is added to a 4 ounce clear glass jar containing 54.14 parts of NeoCryl BT-520 and 0.43 parts of Instant Stabilizer under vigorous agitation and is agitated for 10 minutes. The lid is placed securely on the jar. The liquid coating samples are visually observed for clarity over time. The development of opacity or haziness is indicative of an incompatibility between the hindered amine stabilizer and the formulated coating.

Liquid coating in jar

| Sample* | 0 | Day 1 | 1 Month |
|---|---|---|---|
| A | clear | clear | clear |
| B | hazy | hazy | hazy |
| C | clear | clear | clear |
| D | clear | clear | clear |
| E | clear | clear | clear |
| F | clear | clear | clear |

*A is unstabilized
B contains 2% by weight of bis(1-octyloxy-2,2,6,6-tetramethylpiperidin-4-yl) sebacate
C contains 2% by weight of Instant Example 111
D contains 2% by weight of Instant Example 107
E contains 2% by weight of Instant Example 105
F contains 2% by weight of Instant Example 109

These data show that the instant compounds provide excellent solubility and compatibility in an alkali-soluble, water-borne acrylic copolymer coating system.

### EXAMPLE 113

Formulation Reference: Bayer Corp., 2K Waterborne Clear, Notebook #820894-A, Technical Brochure.

The following components are mixed together under good agitation:

| | |
|---|---|
| Bayhydrol VP LS 2235 | 51.66 parts |
| Deionized Water | 15.70 parts |
| Borchigel LW 44 | 0.24 parts |
| Instant Stabilizer | 1.15 parts |
| UVA | 0.73 parts |
| Baysilone Paint Additive VP AI 3468 | 0.28 parts |

UVA is 2-(3-t-butyl-2-hydroxy-5-(2-(ω-hydroxy-octa-(ethyleneoxy)carbonyl-ethyl)-, phenyl)-2H-benzotriazole, a benzotriazole based UV absorber.

This solution is added to a 4 ounce clear glass jar containing 15.56 parts of Bayhydur XP-7165 and 3.91 parts of Exxate 700 under vigorous agitation and is agitated for 10 minutes. The lid is placed securely on the jar. The solidified coating samples are visually observed for clarity over time. The development of opacity or haziness is indicative of an incompatibility between the hindered amine stabilizer and the formulated coating.

Solidified coating in jar

| Sample* | 0 | Day 1 | 1 Month |
|---|---|---|---|
| A | clear | clear | clear |
| B | hazy | hazy | hazy |
| C | clear | clear | clear |
| D | clear | clear | clear |
| E | clear | clear | clear |
| F | clear | clear | clear |

*A is unstabilized
B contains 2% by weight of bis(1-octyloxy-2,2,6,6-tetramethylpiperidin-4-yl) sebacate
C contains 2% by weight of Instant Example 111
D contains 2% by weight of Instant Example 107
E contains 2% by weight of Instant Example 105
F contains 2% by weight of Instant Example 109

These data show that the instant compounds provide excellent solubility and compatibility in a two component, water-borne polyurethane coating system.

### EXAMPLE 114

The hindered amine test stabilizers are incorporated into a coating system as described in Example 112. A 9.5-10 gram sample of each of the liquid coatings is added to a 100 mm×15 mm Petri dish and is placed in an oven at 65C. for thirty minutes. The samples are taken out of the oven, allowed to cool, and are visually observed for clarity over time. The development of opacity or haziness is indicative of an incompatibility between the hindered amine stabilizer and the formulated coating.

Solidified coating in Petri Dish

| Sample* | 0 | Day 1 | 1 Month |
|---|---|---|---|
| A | clear | clear | clear |
| B | hazy | hazy | hazy |
| C | clear | clear | clear |
| D | clear | clear | clear |
| E | clear | clear | clear |
| F | clear | clear | clear |

*A is unstabilized
B contains 2% by weight of bis(1-octyloxy-2,2,6,6-tetramethylpiperidin-4-yl) sebacate
C contains 2% by weight of Instant Example 111
D contains 2% by weight of Instant Example 107
E contains 2% by weight of Instant Example 105
F contains 2% by weight of Instant Example 109

These data show that the instant compounds provide excellent solubility and compatibility in an alkali-soluble, water-borne acrylic copolymer coating system.

### EXAMPLE 115

The hindered amine test stabilizers are incorporated into a coating system as described in Example 113. A 9.5-10 gram sample of each of the liquid coatings is added to a 100 mm×15 mm Petri dish and is placed in an oven at 75C. for 120 minutes. The samples are taken out of the oven, allowed to cool, and are visually observed for clarity over time. The development of opacity or haziness is indicative of an incompatibility between the hindered amine stabilizer and the formulated coating.

Solidified coating in Petri Dish

| Sample* | 0 | Day 1 | 1 Month |
|---|---|---|---|
| A | clear | clear | clear |
| B | hazy | hazy | hazy |
| C | clear | clear | clear |
| D | clear | clear | clear |
| E | clear | clear | clear |
| F | clear | clear | clear |

*A is unstabilized
B contains 2% by weight of bis(1-octyloxy-2,2,6,6-tetramethylpiperidin-4-yl) sebacate
C contains 2% by weight of Instant Example 111
D contains 2% by weight of Instant Example 107
E contains 2% by weight of Instant Example 105
F contains 2% by weight of Instant Example 109

These data show that the instant compounds provide excellent solubility and compatibility in a two component, water-borne polyurethane coating system.

### EXAMPLE 116

### Two-Component Acrylic Urethane Clearcoat

The hindered amine test stabilizers are incorporated into a two-component acrylic urethane coating as described in Example 100. The system is catalyzed with 0.02% by weight of dibutyltin dilaurate based on the total resin solids. The stabilizers are added at the appropriate level to the acrylic polyol portion of the two-component coating which is then combined with the isocyanate component immediately prior to application.

Steel panels 3″×4″ primed with an electrocoat primer are then coated with a light blue metallic basecoat, then with the stabilized clearcoat. The basecoat is spray applied to a thickness of 1.0 mil (25 microns) dry film thickness and the stabilized clearcoat is then applied to a thickness of 2.0 mils (50 microns) dry film thickness. The coating is air-dried and aged for two weeks. The panels are then exposed in a Xenon-Arc Weather-Ometer under the following conditions:

Cam 180 cycle: 40 minutes light only; 20 minutes light and front spray; 60 minutes light only; 60 minutes dark and rear spray condensate.

Lamp filters are: quartz inner/borosilicate S outer.

Irradiance: 0.45 watts per square meter.

20° Gloss is measured before exposure and at 500 hour intervals during exposure. Higher gloss retention is desirable.

The instant compound are quite efficacious with regard to the retention of 20° Gloss values.

### EXAMPLE 117

### Coatings over Plastic Substrates

A major application for non-basic hindered amines is in the protection of automotive topcoats applied over plastic substrates. However, many low molecular weight, non-reactable light stabilizers migrate into the plastic substrate during drying and cure. As a consequence, a significant portion of the light stabilizer may be lost from the topcoat into the substrate and hence be ineffective in protecting said topcoat.

The extent of migration of hindered amine stabilizers during application and cure of the coating is determined by comparing the concentration of hindered amine in the cured clearcoat applied over a plastic substrate versus the same clearcoat applied over a non-permeable substrate such as glass or steel.

Hindered amine stabilizers under test are incorporated into a flexible thermoset acrylic/melamine clear coating appropriate for use on automotive plastic substrates. The hindered amine is incorporated at a level of 1.5% by weight based on total resins solids.

Each coating formulation is applied by an automatic spray apparatus onto automotive grade RIM (Reacting Injection Molded) substrate and TPO (thermoplastic polyolefin). Both substrates are in form of 4″×12″ plaques. Each coating is applied to achieve a dry film thickness of approximately 2.0 mils (50 microns). The coatings are cured by baking at 250° F. (121° C.) for 20 minutes.

Triplicate samples of each cured coating formulation are removed from each substrate and cryoground to a fine powder. A known amount of each sample is extracted in refluxing toluene overnight. The hindered amine present is analyzed quantitatively by dilution to a known volume and analyzed by HPLC or SFC chromatography. Calibration curves for each test stabilizer compound are developed. The hindered amine content of each extracted coating is determined by this method.

When the instant hindered amine compounds are compared to other hindered amines, for example bis(1-octyloxy-2,2,6,6-tetramethylpiperidin-4-yl) sebacate, a higher percent recovery of the instant hindered amine compound from the clearcoat over a plastic substrate is found indicating that much less of the instant hindered amine stabilizer migrates into the plastic substrate allowing for better stabilization of the clear topcoat over such plastic substrates.

### EXAMPLE 118

### Stabilization of Water Borne Wood Varnish

Waterborne coatings comprise a significant and increasing proportion of the coating in use for a wide variety of applications including automotive basecoats, industrial coatings and trade sale coatings. These coatings may be pigmented or transparent. The trends are also towards higher solids formulation which in general depend on light stabilizers to maintain properties on exterior exposure, and towards lower levels of co-solvents. This requires higher solubility of stabilizers in such co-solvents (primarily water) or actual solubility in water.

The test stabilizers are incorporated into a waterborne dispersion by predissolution in a co-solvent blend. The waterborne dispersion is a commercially available acrylic/urethane hybrid resin. The co-solvent blend is a 1:1 mixture of TEXANOL® (2,2,4-trimethyl-1,3-pentanediol, Texaco) and ARCOSOLVE® TPM (tripropylene glycol methyl ether, AtlanticRichfield).

0.45 gram of the test stabilizer is predissolved in 10 g of the co-solvent blend which is then incorporated into the following composition:

| | ppw |
|---|---|
| FLEXTHANE ® 630 (Air Products) | 100.0 |
| Foamaster VF | 0.1 |
| Water | 10.0 |
| TEXANOL/ARCOSOLVE/hindered amine | 10.5 |
| UV absorber | 1.2 |
| BYK 346 | 0.5 |
| MICHEMLUBE ® 162 | 2.0 |

UV absorber is 2-(3-t-butyl-2-hydroxy-5-(2-(ω-hydroxy-octa-(ethyleneoxy)carbonyl-ethyl)-phenyl)-2H-benzotriazole, a benzotriazole based UV absorber.

Each coating is brush applied onto 6″×6″ sections of cedar and pine boards. Weighing the coating and brush before and after application and ensuring that the same weight of coating is applied to each section regulates the weight of the coating applied.

The coated board sections are allowed to dry at ambient temperature for two weeks, then evaluated for visual appearance, gloss and Hunter L*, a* and b* color. The sections are exposed on racks at a 45° angle in South Florida for six months before being returned and evaluated for visual appearance, gloss, color change and any other signs of degradation or delamination.

The instant hindered amine compounds provide good stabilization efficacy to the sections in respect to visual appearance, gloss retention, resistance to color change and to delamination.

### EXAMPLE 119

### Stabilization of Pigmented Automotive OEM Basecoat

A basecoat pigmented with a mixture of Pigment Red 177 and mica is stabilized with 1% by weight of a hindered amine stabilizer based on the total basecoat solids (pigment plus resin). The basecoat is spray applied at a dry film thickness of 1 mil (25 microns) to primed 4″×12″ steel panels, then topcoated with a high solids commercially available automotive clearcoat. The coated panels are cured in an over at 250° F. (121° C.) for 30 minutes. The panels are then exposed in a Xenon-Arc Weather-Ometer under the following conditions:

Cam 180 cycle: 40 minutes light only; 20 minutes light and front spray; 60 minutes light only; 60 minutes dark and rear spray condensate.

Lamp filters are: quartz inner/borosilicate S outer.

Irradiance: 0.55 watts per square meter.

20° Gloss, Distinctness of Image, Hunter Color Space Values (L*, a*, b* and DE) are measured before exposure and after 3000 hours of exposure.

The instant hindered amine compounds provide good stabilization efficacy to the panels in respect to distinctness of image, gloss retention and resistance to color change.

### EXAMPLE 120

Thermoplastic materials composed of mixtures of copolymers derived from the copolymerization of styrene monomer with acrylonitrile and the copolymerization of styrene monomer with butadiene, generally referred to as ABS, are dry blended with the instant compounds and melt compounded into pellets. Typical formulations contain the instant compounds at levels from 0.05% to 2.0%, a metal stearate such as calcium stearate at 0.05% to 0.5%, pigments from 0% to 5%, UV absorbers at levels of 0.05% to 2.0%, phosphites at 0.0%-0.1%, phenolic antioxidants at 0.0%-1.25%, N,N-dialkylhydroxylamine at 0.0%-0.1%, and optionally other hindered amine stabilizers at levels of 0.0% to 2.0%.

The pelletized fully formulated resin is then processed into a useful article such as extrusion into sheet, film, profile and pipe; molded into bottles; injection molded into a molded article; thermoformed into molded articles; or rotational molded into hollow articles.

The materials containing the instant compounds exhibit stability against deleterious effects of UV light and thermal exposure.

### EXAMPLE 121

Fiber grade polypropylene, is dry blended with the test additives and then melt compounded at 234° C. (450° F.) into pellets. All formulations additionally contain melt processing stabilizer system. The pelletized fully formulated resin is then spun at 246° C. (475° F.) into fiber using a Hills laboratory model fiber extruder. The spun tow of 41 filaments is stretch at a ratio of 1:3.2 to give a final denier of 615/41.

The fibers are then knitted into socks and on a Lawson-Hemphill Analysis Knitter. Ten replicates of each sample are tested under NFPA701-1996 Vertical burn procedure. The time in seconds for the knitted sock to extinguish after the insult flame is removed is reported as “After Flame”. Efficacy as a flame retardant is demonstrated when low After Flame times are observed relative to a blank sample containing no flame retardant. The burning time of the drips from the material and the weight loss are also recorded. The instant stabilizers are effective as flame retardants.

### EXAMPLE 122

Molding grade polypropylene is dry blended with test additives and then melt compounded into pellets. In addition to the instant compound, halogenated flame retardants are included in the formulation. Typical formulations contain the instant compound and a flame retardants such as: tris(3-bromo-2,2 bis (bromomethyl)propyl) phosphate (FMC PB370); bis(2,3-dibromopropyl ether) of bisphenol A (PE68); decabromodiphenyloxide (DBDPO); ethylene bis-tetrabromophthalimide (SATEX BT-93); ethylene bis-dibromonorbornanedi-carboximide (SATEX BN-451). Other formulations may contain Sb₂O₃ in addition to the brominated flame retardants. Other formulations may contain phosphorous based flame retardants such as ethylene diamine diphosphate (EDAP). The pelletized fully formulated resin is then compression molded into test specimens using a Wabash Compression Molder.

Test plaques are tested under UL-94 Vertical burn conditions. A minimum of three replicates are tested. The average time in seconds for the test sample to extinguish after a first and second insult flame is removed is reported. Efficacy as a flame retardant is demonstrated when low Flame times are observed. The instant compounds enhance the flame retardancy of a halogenated or phosphate flame retardant tested alone.

### EXAMPLE 123

Molded test specimens were prepared by injection molding thermoplastic olefin (TPO) pellets containing the instant compounds. The TPO formulations may also contain pigments, a phenolic antioxidant, phosphite or hydroxylamine, a metal stearate, ultraviolet light absorbers (UVA) or a hindered amine stabilizers (HALS) or a mixture of UV absorbers and hindered amine stabilizers.

In addition to the instant compound, halogenated flame retardants are included in the formulation. Typical formulations contain the instant compound and a flame retardants such as: tris(3-bromo-2,2 bis (bromomethyl)propyl) phosphate (FMC PB370); bis(2,3-dibromopropyl ether) of bisphenol A (PE68); decabromodiphenyloxide (DBDPO); ethylene bis-tetrabromophthalimide (SATEX BT-93); ethylene bis-dibromonorbornanedi-carboximide (SATEX BN-451). Other formulations may contain Sb₂O₃ in addition to the brominated flame retardants. Other formulations may contain phosphorous based flame retardants such as ethylene diamine diphosphate (EDAP).

Test plaques are tested under UL-94 Vertical burn conditions. A minimum of three replicates are tested. The average time in seconds for the test sample to extinguish after a first and second insult flame is removed is reported. The instant compounds enhance the flame retardancy of a halogenated or phosphate flame retardant tested alone.

### EXAMPLE 124

Molding grade ABS is dry blended with test additives and then melt compounded into pellets. In addition to the instant compounds, selected flame retardants are also included. The flame retardants are tris[3-bromo-2,2-bis(bromomethyl)propyl] phosphate, decabromodiphenyl oxide, ethylene bis(tetrabromophthalimide) and ethylene bis(dibromonorbornanedicarboximide). The pelletized fully formulated resin is then injection molded into test specimens using a BOY 50M laboratory model injection molder. Other formulations may contain antimony trioxide (Sb₂O₃) in addition to the brominated flame retardants. Other formulation may contain phosphorus based flame retardants such as ethylenediamine diphosphate (EDAP).

Test plaques are mounted in metal frame and exposed in an Atlas Ci65 Xenon Arc Weather-O-meter with intermittent light/dark cycles and water spray following the ASTM G26 test procedure. Specimens are tested at periodic intervals for changes in tensile properties and for changes in color. The longer it takes for the loss in properties to occur and the less the color change as measured by DE, the more effective is the stabilizer system.

The test samples containing the instant compounds exhibit good retention of tensile properties and minimal color change during the accelerated weathering.

### EXAMPLE 125

Molding grade high impact polystyrene is dry blended with test additives and then melt compounded into pellets. In addition to the instant compounds, selected flame retardants are also included. The flame retardants are tris[3-bromo-2,2-bis(bromomethyl)propyl] phosphate, decabromodiphenyl oxide, ethylene bis(tetrabromophthalimide) and ethylene bis(dibromo-norbornanedicarboximide). The pelletized fully formulated resin is then injection molded into test specimens using a BOY 50M laboratory model injection molder. Other formulations may contain antimony trioxide (Sb₂O₃) in addition to the brominated flame retardants. Other formulation may contain phosphorus based flame retardants such as ethylenediamine diphosphate (EDAP).

Test plaques are mounted in metal frame and exposed in an Atlas Ci65 Xenon Arc Weather-O-meter with intermittent lightdark cycles and water spray following the ASTM G26 test procedure. Specimens are tested at periodic intervals for changes in tensile properties and for changes in color. The longer it takes for the loss in properties to occur and the less the color change as measured by DE, the more effective is the stabilizer system.

The test samples containing the instant compounds exhibit good retention of tensile properties and minimal color change during the accelerated weathering.

### EXAMPLE 126

### High Solids Acid-Catalyzed Thermoset Acrylic Resin Enamel

A high solids (50% by weight) thermoset acrylic resin enamel, catalyzed by 0.8% by weight of dodecylbenzenesulfonic acid, based on the film-forming resin is stabilized by the addition of various instant compounds. The high solids thermoset acrylic resin enamel formulation (Acryloid AT 400 from Rohm and Haas) is based on hydroxyethyl methacrylate, methyl methacrylate, styrene, butyl acrylate and butyl methacrylate and a melamine curing agent.

Pieces of steel sheeting 4″×12″ (9.16 cm×30.48 cm), coated with a primer based on polyester/epoxy resin, are then coated with a TiO₂-pigmented base coat based on a binder of 70% of monomers such as hydroxyethyl acrylate, styrene, acrylonitrile, butyl acrylate and acrylic acid with 30% of a melamine resin and an acid catalyst and finally with a clear finishing enamel. The base coat is sprayed onto the sheet to a thickness of about 0.8 mil (0.0203 mm) and air dried for three minutes. The clear finishing enamel is then sprayed onto the sheet to a thickness of about 2.0 mil. After 15 minutes air-drying, the coated sheets are baked for 30 minutes at 121° C.

The stabilizers under test are added to the thermoset acrylic resin finishing enamel in a concentration of 1% by weight before the enamel is coated onto the base coat.

The coated sheets, after storage for three weeks in an air-conditioned room (23° C./50% relative humidity), are subjected to weathering for 2000 hours according to SAE J1920 in a Xenon arc Weather-Ometer. In this apparatus, samples are subjected to weathering in repeated cycles of 180 minutes. The effectiveness of the stabilization is measured by the retention of 20° gloss after weathering.

The sheets stabilized by the instant compounds exhibit good retention of 20° gloss after weathering under extreme weather conditions.

### EXAMPLE 127

The samples prepared in Example 126 are also evaluated on the basis of Knoop Hardness (ASTM D-1474-68) on baked and overbaked samples; on the distinction of image (DOI); on Hunter Associates Apparatus; on 20° gloss (ASTM D-523-80); and on cracking based on visual observation.

The samples stabilized by the instant compounds exhibit a pattern of greater retention of 20° gloss and DOI, and a longer absence of severe cracking after exposure.

### EXAMPLE 128

The thermoset acrylic enamel of Example 126 is formulated to include 3% by weight of a benzotriazole UV absorber and 1.5% by weight of an instant hindered amine test compound. The enamel is coated over a white base coat or over a silver metallic base coat. Baking is conducted at 121° C. normal bake or at 82° C. automotive low bake repair temperature.

The coated panels are exposed in a Xenon arc exposure apparatus and 20° gloss and distinction of image (DOI) values are determined.

The samples stabilized by the instant compounds exhibit a pattern of greater retention of 20° gloss and DOI.

### EXAMPLE 129

### Thermoset Acrylic Enamel

Two thermoset acrylic enamels are formulated to include 3% by weight of a benzotriazole UV absorber and 1% by weight of an instant hindered amine test stabilizer.

The thermoset acrylic enamels are based on a binder of 70% of monomers such as hydroxyethyl acrylate, styrene, acrylonitrile, butyl acrylate and acrylic acid with 30% of a melamine resin and an acid catalyst such as p-toluenesulfonic acid, dinonylnaphthalene-disulfonic acid, dodecylbenzenesulfonic acid or phenyl acid phosphate.

Pieces of steel sheeting 4″×12″ (9.16 cm×30.48 cm), coated with a primer based on polyester/epoxy resin, are then coated with a base coat and finally with a clear finishing enamel. The base coat is sprayed onto the sheet to a thickness of about 0.8 mil (0.0203 mm) and air dried for three minutes. The clear finishing enamel is then sprayed onto the sheet to a thickness of about 2.0 mil. After 15 minutes air-drying, the coated sheets are baked for 30 minutes at 121° C.

The coated panels are exposed in a Xenon arc exposure apparatus and 20° gloss and distinction of image (DOI) values are determined.

The samples stabilized by the instant compounds exhibit a pattern of greater retention of 20° gloss and DOI.

### EXAMPLE 130

### Coil Coating

A white polyester/melamine based oil-free alkyl coil coating is utilized in this example. The fully formulated paint is applied over a primed steel sheet using a wire wound rod to give 0.6-0.8 mil dry film. The panels are baked for about 90 seconds at 220° C., removed from the oven and immediately quenched in water. The coated panels are exposed in a Xenon Arc Weather-Ometer, and in South Florida at an angle of 45° S to the sun. 20° gloss values are determined.

The samples stabilized by the instant compounds exhibit a pattern of greater retention of 20° gloss.

### EXAMPLE 131

The thermoset acrylic enamel of Example 130 including 0.8% dodecylbenzenesulfonic acid is formulated to include varying concentrations of benzotriazole or s-triazine UV absorbers and the instant hindered amine test compounds. The enamel is coated over a silver metallic base coat pursuant to the procedure of Example 130 and baking is conducted for 30 minutes at 121° C. the normal bake temperature.

The coated panels are exposed in a Xenon arc Weather-Ometer and the time to the 50% loss of 20° gloss is determined.

The samples stabilized by the instant compounds and a UV absorber exhibit an excellent retention of 20° gloss and a much longer time till 50% loss in 20° gloss is observed.

### EXAMPLE 132

A thermoset acrylic enamel based on a binder of 70% of monomers such as hydroxyethyl acrylate, styrene, acrylonitrile, butyl acrylate and acrylic acid with 30% of a melamine resin and an acid catalyst such as p-toluenesulfonic acid, dinonylnaphthalenedisulfonic acid or dodecyl-benzenesulfonic acid is formulated. Commercially available 9.16 cm×30.48 cm Uniprime panels are used as the substrate. The panels are coated with a silver metallic base coat and then with a clear finishing enamel. The base coat is stabilized with 1% of a benzotriazole UV absorber and 1% of an instant hindered amine test compound (based on solid resin) and is sprayed onto the panel to a thickness of about 0.6-0.8 mil and air dried for three minutes. The clear coat including the above-noted stabilizers is then sprayed to a thickness of 1.7-2.0 mils and after 10 minutes of air drying, the coated panels are baked for 30 minutes at 121° C. The coated panels are then exposed in a Xenon arc apparatus and the 20° gloss values are determined.

The samples stabilized by the instant compounds and a UV absorber exhibit excellent retention of 20° gloss.

### EXAMPLE 133

### Water Borne Acrylic Melamine Enamel

A water-borne acrylic melamine enamel is formulated as seen below:

| | Parts Resin Solids |
|---|---|
| Synthacryl VSW 6483 | 30 |
| (acrylic dispersion from Hoechst) | |
| Synthacryl VSW 6484 | 42 |
| (50% acrylic resin in butyl diglycol, Hoechst) | |
| Maprenal MF 915 | 25 |
| (70% melamine resin in isobutanol) | |
| Maprenal MF 927 (melamine resin) | 3 |
| | 100 |

A water-based base coat/clear coat enamel is prepared by spray applying a 0.6-0.8 mil thick film of commercial silver metallic waterborne base coat (from BASF) over an epoxy primed coil coated aluminum panel. This material is baked at 80° C. for five minutes and then clear coated with 1.6-1.8 mil of the waterborne enamel. The system is baked at 80° C. for ten minutes and then at 140° C. for a further 30 minutes. Prior to application of the clear coating, instant test and light stabilizers dissolved in a minimum amount of butyl glycol acetate are added to the paint. The coated panels are exposed in a Xenon arc apparatus for 975 hours. The distinction of image (DOI) retention of the panels is measured.

The samples stabilized by the instant compounds exhibit greater retention of DOI values.

### EXAMPLE 134

Pieces of 1.27 cm×20.32 cm×30.48 cm western red cedar panels having a fine radial cut are used to test a commercially available tung oil phenolic varnish (supplied by McCloskey). One half of each panel is coated with two coats of unstabilized varnish. An equal amount of varnish containing 5% by weight (based on resin solids) of test stabilizers is applied to the other half of the panel in two coats. After storage for two weeks at ambient temperature, the wood panels are exposed outdoors at an angle of 45° S for a period of eight months. The 60° gloss of each half of the panel is measured at the top, middle and bottom portion of the panel and averaged according to ASTM D 523. Due to the lack of homogeneity of wood substrates, the gloss retention of the same varnish tends to differ slightly from panel to panel. Thus, the application of an unstabilized control varnish to every panel allows for a more meaningful measurement of the improvement in gloss due to the presence of the instant test compound.

The panels stabilized by the instant compounds show excellent gloss retention after long exposure.

### EXAMPLE 135

### Aromatic Urethane Varnish

A sample of commercial aromatic urethane varnish (Flecto-Varathane #90) is tested by the same method described in Example 134. After outdoor exposure at an angle of 45° S for a period of five months, the 60° gloss retention values of unstabilized and stabilized portions of the panels are determined.

The panels stabilized by the instant compounds show excellent gloss retention.

### EXAMPLE 136

### White Two-Component Polyester Urethane Gloss Enamel

A white polyester is formulated as shown below:

| | Parts |
|---|---|
| Component I | |
| Desmophen 670-90 (polyester glycol, Mobay) | 132.4 |
| Titanium Dioxide | 198.6 |
| Cellosolve Acetate | 98.9 |
| Sand Mill | |
| Desmophen 670-90 | 94.98 |
| Flow Aid | 0.28 |
| Tertiary Amine | 0.015 |
| Cellosolve Acetate | 332.6 |
| Component II | |
| Desmodur N-100 (polyisocyanate, Mobay) | 93.9 |
| Cellosolve Acetate | 58.7 |

This material is spray applied at a dry film thickness of 1.5-2.0 mil onto Bonderite 40 cold rolled steel panels that have been previously primed with a commercial epoxy polyamide maintenance primer (Sherwin-Williams Tile Clad II). Prior to application, the instant test compounds are added to the paint. After ambient storage for two weeks, three panels of each formulation are exposed outdoors at an angle of 45° S for a period of nine months. Thereafter, 20° gloss retention is determined by ASTM D 523-80 at the top, middle and bottom portions of each panel. Thus, the average values for nine gloss retention measurements for each triplicate set of panels are obtained.

The panels stabilized by the instant compounds show excellent gloss retention.

### EXAMPLE 137

### Acrylic Alkyd Refinish Enamel

A commercially available acrylic alkyd enamel pigments with non-leafing aluminum pigment and tinted a light blue is stabilized with a benzotriazole UV absorber and an instant hindered amine test compound and is then spray applied onto Bonderite 40 panels primed with an alkyd primer. After the coating is allowed to cure at room temperature for 14 days, the panels are exposed outdoors at an angle of 45° S for a period of eight months. The 20° gloss of the exposed panels is measured.

The panels stabilized by the instant compounds show excellent gloss retention.

### EXAMPLE 138

### Medium Oil Alkyd Enamel

A medium oil alkyd enamel pigmented with a non-leafing aluminum pigment and tinted light blue is stabilized with a benzotriazole UV absorber and an instant hindered amine test compound and is then sprayed applied onto cold rolled steel panels primed with an epoxy primer. After the coating is allowed to cure at room temperature for two weeks, the panels are exposed for accelerated weathering in a Xenon Arc Weather-Ometer for 840 hours. The 20° gloss values of the panels are determined before and after exposure.

The panels stabilized by the instant compounds show excellent gloss retention.

### EXAMPLE 139

### Electrocoat Composition

A typical E-coat composition is prepared by adding the diglyicidyl ether of bisphenol A, polyethylene oxide diol, bisphenol A and xylene to a flask and heating to 135° C. The catalyst dimethylbenzylamine in xylene is added and the temperature maintained at 143° C. for two hours. The weight per epoxy (WPE) is measured and a previously prepared crosslinker composed of 2,4-toluenediisocyanate, trimethylolpropane blocked with an alcohol is then added and temperature reduced to 100° C. The remaining epoxy groups are then capped with two different secondary amines, namely diketimine of diethylenetriamine and methylethanolamine, in phenyl cellosolve. The temperature is maintained for one hour at 110° C. and the crosslinker hexamethylenediisocyanate blocked with an alcohol is added. The temperature is maintained near 100° C. for 30 minutes and the resin mixture is added to deionized water, surfactant and lactic acid to give a resin emulsion.

To this resin emulsion is added the instant hindered amine compound, additional epoxy resin, carbon black, dibutyltin oxide catalyst, titanium dioxide, lead silicate, water and UV absorber. After dispersion using a sand mill to achieve proper fineness, the mixture is incorporated into an electrocoat bath with water for electrocoating onto a metal substrate.

The steel coating electrocoated with the above E-coat resin composition to a thickness of 23-30 mm and cured for 20 minutes at a temperature of 176-201° C. A pigmented resin layer is coated thereover at a thickness of 20-51 mm using an acrylic coating composition in an organic solver, pigments and a UV absorber. The coated panels are then baked at 121-129° C. to cure the pigmented layer.

The panels are then exposed outdoors for four months. The panels containing the instant hindered amine compound, particularly when used with a UV absorber, provided excellent resistance to delamination of the E-coat layer from the metal substrate.

### EXAMPLE 140

### Abrasion Resistant Coating Compositions

A solution in isopropanol of 50% (by weight) of 1,6-hexanediol, 10% 3-methacryloyloxy-propyltrimethoxysilane and 40% colloidal silica (in form of a 34% aqueous dispersion) is vacuum stripped to remove volatiles and combined with an instant hindered amine compound; a benzotriazole UV absorber and 2,4,6-trimethylbenzoyldiphenylphosphine photoinitiator. These compositions show no gelation on storage.

The compositions above are applied by roller coating to a 15 mil film of bisphenol A polycarbonate and the coated films are passed under a mercury lamp at 43° C. at a line speed of 610 cm/min. The compositions are cured to a colorless and optically clear coatings over the polycarbonate substrate.

The coatings as measured by the Taber Abrasion Test (ASTM D1044) are abrasion resistant.

The test specimens are also subjected to accelerated aging tests using an Atlas Ci35A Xenon Arc Weather-Ometer. The results show that the coatings containing the instant hindered amine compound exhibit excellent resistance to yellowness and haze formation.

### EXAMPLE 141

### Coating over Polycarbonate

A two-component polyester urethane coating is stabilized by the addition of an instant hindered amine compound. The high-solids polyester polyol (Desmophen 670-80, Bayer) is crosslinked with an isocyanate based resin (Desmodue N-3390, Bayer). The coating is catalyzed with 0.015% by weight of dibutyltin dilaurate catalyst.

Plaques of polycarbonate-based plastic substrate (Xenoy) 4″×6″ are coated with the formulated clear coat at a thickness of approximately 1.5 mils. The coating is spray applied to the substrate and then baked at 82° C. for 20 minutes.

After storage for one week at room temperature, each plaque is cut into 2″×3″ strips with five replicates being made for each formulation. Each strip is placed into a 8-oz jar along with 2 mL of distilled water and sealed. All samples are placed in an over at 54° C. A crosshatch adhesion test is performed once a week on at least two of the replicate samples until the sample failed (5% adhesion loss) or until 40 days elapses.

The samples containing the instant hindered amine compounds exhibit excellent resistance to delamination.

### EXAMPLE 142

### Photographic Layers

Chromogenic photographic layers are prepared by hand-coating a gelatine emulsion containing silver bromide, yellow coupler and an additive on a polyethylene-coated paper.

The composition of the layer is as given in following table, amounts are in mg/m²:

| Component | Amount in the layer |
|---|---|
| Gelatine | 5150 |
| AgBr | 520 |
| Yellow coupler | 1.07 mmol/m² |
| Coupler solvent solv1 | 33% of the coupler weight/m² |
| Additive | 30% of the coupler weight/m² |
| Hardener ha1 | 300 |
| Surfactant su1 | 340 |

The layers are dried for 7 days in a ventilated cabinet.

The dried samples are exposed to white light through a stepwedge of 0.3 logE exposure steps. They are developed with the P94 process for negative color paper from Agfa-Gevaert, following the manufacturers' recommendations.

After exposure and processing, the remission density of the yellow dye is measured in the blue channel. The samples are then exposed in an Atlas WeatherOmeter behind a separate UV filter so as to receive 60 kJ/cm² light energy. The UV filter consists of an emulsion coated onto a polyester transparent support, such that the layer contains 1 g/m² of a benzotriazole UV absorber. The temperature is 43° C. and the relative humidity 50%. The density loss starting from a blue-density of 1 is determined. Low ΔD number are desired.

The additives of the present invention improve the light stability of yellow photographic layers.

### EXAMPLE 143

### Photographic Layers

Chromogenic photographic layers are prepared by hand-coating a gelatine emulsion containing silver bromide, yellow coupler and an additive on a polyethylene-coated paper.

The composition of the layer is as given in following table, amounts are in mg/m²:

| Component | Amount in the layer |
|---|---|
| Gelatine | 5150 |
| AgBr | 520 |
| Yellow coupler | 1.07 mmol/m² |
| Coupler solvent solv1 | 33% of the coupler weight/m² |
| Additive | 30% of the coupler weight/m² |
| Hardener ha1 | 300 |
| Surfactant su1 | 340 |

The layers are dried for 7 days in a ventilated cabinet.

The dried samples are exposed to white light through a stepwedge of 0.3 logE exposure steps. They are developed with the P94 process for negative colour paper from Agfa-Gevaert, following the manufacturers recommendations.

After exposure and processing, the remission density of the yellow dye is measured in the blue channel. The samples are then subjected to storage in a Weiss climatic cabinet for 28 days. The density loss starting from a blue-density of 1 is determined. Low DD numbers are desired.

The additives of the present invention improve the dark stability of yellow photographic layers.

### EXAMPLE 144

Molded test specimens are prepared and tested as in Example 77 replacing N,N-dialkylhydroxylamine base stabilizer in the samples with the amine oxide, Genox™ EP, a di(C₁₆-C₁₈)alkyl methyl amine oxide, CAS# 204933-93-7. Light stabilizer formulations comprising a mixture of components outlined in Example 77, an amine oxide and the instant compounds exhibit good stabilization performance against deleterious effects of UV light.

### EXAMPLE 145

Molded test specimens are prepared as in Example 78 replacing N,N-dialkylhydroxylamine base stabilizer in the samples with the amine oxide, Genox™ EP. Formulations comprising a mixture of components outlined in Example 77, an amine oxide and the instant compounds exhibit good paint adhesion compared with a formulation substituting the close prior art compound where no hydroxyl moiety is present for the instant compounds.

### EXAMPLE 146

Fiber specimens are prepared and tested as in Example 80 replacing N,N-dialkyihydroxylamine base stabilizer in the samples with the amine oxide, Genox™ EP. Light stabilizer formulations comprising a mixture of components outlined in Example 79, an amine oxide and the instant compounds exhibit good stabilization performance against deleterious effects of UV light.

### EXAMPLE 147

### Glycidyl Methacrylate-Based Powder Clearcoat

One of the major new coating technologies that can be used to meet increasingly stringent VOC solvent emission requirements is the use of powder coatings. Applications requiring the use of light stabilizers include clearcoats for finishing of automotive topcoats, finishing of garden implements, protection of automotive wheel covers. For optimum incorporation and shelf stability, stabilizers used in a powder coating should be moderate-melting (˜100° C.) solids, nonvolatile, and heat stable at typical powder coating baking temperatures (140-180° C.).

Prior to incorporation of the Hindered Amine Light Stabilizers under test, a premix of commercially available GMA-based powder coating resin, UV absorber, and flow aids is made by extruding together at 145° C. The Hindered Amine Light Stabilizers under test are then incorporated into portions of this premix, along with a commercially available 1,12 dodecanoic acid crosslinking resin. The final mix is extruded at 100° C., then the extrudate is milled on an ultracentrifugal mill and powder cyclone, and sieved. The powders are electrostatically sprayed onto a basecoat to a film thickness of 60 microns. The coatings are cured for 30 minutes at 160° C.

The panels are weathered in a Xenon WeatherOmeter, and in Florida at 5° South angle. Gloss and color are measured.

The panels stabilized by the instant compounds show excellent gloss retention.

### EXAMPLE 148

### Oil Modified Urethane Alkyd for Wood Application

The Hindered Amine Light Stabilizers under test are incorporated into a commercially available solvent-borne urethane alkyd McWhorter 43-4355. A 2-hydroxy benzotriazole UV absorber is also incorporated into all formulations. The Hindered Amine light stabilizers are added at equivalent piperidine levels. After mixing, the clear coatings are applied by brushing to white pine boards. Each board is divided into 8 sections separated by a groove ⅛″ deep, which is sealed with a film-forming clear varnish and the back and sides are coated with a white chlorinated pool paint. The stabilized coatings are applied in triplicate in three coats to sections of the boards in such a way as to ensure that a control formulation is present on each of the boards for comparison. The wood samples are allowed to dry for 1 week, then placed on exposure in Florida, Australia, and New York.

The panels stabilized by the instant compounds show good color retention, cracking resistance, and visual gloss retention.

### EXAMPLE 149

### Preformed Films for Lamination to Plastic Parts

The instant invention also pertains to protective and decorative films which are preformed, then applied to a substrate via a dry paint transfer process. These films consist of a single decorative layer which is applied to a carrier sheet, then laminated to a self-supporting, thermoformable backing sheet. The carrier sheet is then removed from the opposite side of the film, exposing the decorative layer. The composite film/backing sheet then is thermoformed to a three-dimensional shape. Additionally, these films may also consist of multiple layers, where, for example, a thermoplastic, thermoformable clearcoat is applied to the carrier sheet, then hardened to form an optically clear film. A color coat is then applied to the exposed face of the clearcoat, and hardened, resulting in a clear coat/color coat paint film supported by the carrier. This composite is then laminated to a thermoformable backing sheet, as above. The carrier sheet is removed, as above, and the composite clearcoat/colorcoat/backing is then thermoformed, as above.

The polymeric resins for the above application must be thermoplastic, and may be fluoropolymer/acrylic blends.

### EXAMPLE 150

1 to 5 g of the instant compounds, 1 g of tris(2,4-di-tert-butylphenyl) phosphite, 0.5 g of pentaerythritol tetrakis (3-(3,5-di-tert-butyl-4-hydroxyphenyl)propionate) and 1 g of calcium stearate are mixed in a turbomixer with 1000 g of polypropylene powder having a melt index of 2.1 (measured at 230° C. and 2.16 Kg). The final concentration of hindered amine is 0.1% to 0.5% in the PP.

The mixture is extruded at 200-220° C. to give polymer granules which are subsequently converted to stretched tapes of 50 microns thickness and 2.5 mm width, using a semi industrial type of apparatus (Leonard-Sumirago(VA)-Italy) and working under the following conditions:

| Extruder temperature: | 210-230° C. |
|---|---|
| Head temperature: | 240-260° C. |
| Stretch ratio: | 1:6 |

The tapes thus prepared are mounted on a white card and expose in Weather-Ometer 65 WR (ASTM D 2565-85) with a black panel temperature of 63° C.

The residual tensile strength is measured, by means of a constant velocity tensometer, on a sample taken after various light exposure times; from this, the exposure time (in hours) required to halve the initial tensile strength (T₅₀) is measured.

The tapes containing the present hindered amines exhibit excellent UV light stability.

Water carry-over is expressed as the rate at which a film or stretched tape can be extruded before water from the water-cooling bath is carried along with the film. The higher the water carry-over value, the faster and more economically a film or stretched tape can be processed. It is well known in the art that additives can have an effect on water carry-over. The films containing the present hindered amines exhibit high production rates.

### EXAMPLE 151

Film grade polyethylene is dry blended with approximately 10% by weight of a test additive of the present invention and then melt compounded at 200° C. into “Masterbatch” pellets. The fully formulated “Masterbatch” pellets are dry blended with polyethylene resin to get the desired final stabilizer concentrations. Typical formulations contain an additive of the present invention at levels from 0.05% to 2.0%, a metal stearate such as calcium stearate at 0.05% to 0.5%, a phosphite at 0% to 0.1%, a phenolic antioxidant at 0% to 1.25%, an N,N-dialkylhydroxylamine at 0% to 0.1% and optionally a hindered amine at 0% to 2.0%. The stabilized fully formulated resin is then blown at 200° C. into a 150 micron thick film on a DOLCI film line.

The resulting films are exposed for 4 hours to 20 liters of an aqueous solution containing 22.5 ppm chlorine. The chlorine is made available via Leslies Fast Dissolving Super Shock—Super Chlorinator (Shock and Algae control) from OLIN Pool Products, Norwalk Conn. This Super Shock is 78% Calcium Hypochlorite is used accordingly to make the 22.5 ppm Cl available. After the 4 hours of the chlorine exposure the samples are rinsed in Distilled water 3 times, and air-dried to prepare them for accelerated weathering. A duplicate sample is exposed to distilled water without the Chlorine. All the dipped samples are exposed for 250 hour intervals in a Weather-O-meter 65 WR (ASTM D 2565-85 -dry) with a black panel temperature of 63° C. After each 250 hour interval of accelerated weathering, the samples are again exposed to the aqueous exposure as above. Failure is defined as the time to a 50% loss of original elongation. This test is designed to simulate exposure to pool chemicals as would be experienced by pool covers.

The films containing the present compounds show good resistance to pool chemicals containing chlorine.

Other polyolefin articles, such as pool hoses, exposed to pool chemicals containing the present compounds show good resistance to pool chemicals containing chlorine.

### EXAMPLE 152

100 parts high density polyethylene are dry blended with 0.4 parts of 1,2-bis(3,5-di-tert-butyl-4-hydroxyhydrocinnamoyl)hydrazine and 0.2 parts of the instant hindered amines. The mixtures are melt compounded into pellets at 230° C. in a Superior/MPM extruder using a 24:1 L/D screw with Maddock mixing head at 60 rpm.

The pelletized polyethylene containing the stabilizer mixtures are compression molded at 400° F. into 10 mil (0.01 inch) thick films with Mylar backing. “Initial oxidation induction time” (OIT) is measured on these test films.

The sample films are then submersed in Witcogel®, available from Witco, a typical hydrocarbon cable filler grease used in telecom cables. The Witco filling compound contains 0.6% thiodiethylene bis[3-(3,5-di-tert-butyl-4-hydroxyphenyl)propionate]. The sample films submersed in the filling compound are exposed in an air oven at 70° C. for 14 days. The samples are then wiped clean of the cable filler grease. “Aged oxidation induction time” is measured on these samples.

OIT testing is accomplished using a differential scanning calorimeter as per ASTM standard test method D3895. The test conditions are: Uncrimped aluminum pan; no screen; heat up to 200° C. under nitrogen, followed by a switch to a 100 milliliter/minute flow of oxygen. Oxidation induction time (OIT) is the time interval between the start of oxygen flow and the exothermic decomposition of the test specimen. OIT is reported in minutes; under the conditions of this test the longer the OIT the more effective the stabilizer mixture is at delaying the onset of oxidative degradation. Relative performance of stabilizer mixtures in grease filled cable applications can be predicted by comparing the initial OIT values, the aged OIT values and the differences between the initial OIT and aged OIT values.

The stabilizer mixtures containing a metal deactivator and the hindered amines provide excellent performance as measured by initial and aged OIT.

### EXAMPLE 153

Unipol®, Union Carbide Corporation, gas phase polypropylene random copolymer with an initial melt flow rate of ca. 2 dg/min is via addition of a dialkylperoxide, controlled rheology modified to have a target melt flow rate of ca. 25 dg/min, an appropriate melt flow rate for injection molding. A clarifier is added at ca. 2200 ppm to enhance the transparency of the molded articles.

The formulations contain either a binary stabilizer system of the hindered hydrocarbyloxyamines and an organophosphorus compound, a binary system of the hindered hydrocarbyloxyamines and one or more compounds selected from the group of hydroxylamine stabilizers, benzofuranone stabilizers and amine oxide stabilizers, or a ternary system of the hindered hydrocarbyloxyamines, one or more compounds selected from the group of hydroxylamine stabilizers, benzofuranone stabilizers and amine oxide stabilizers and an organophosphorus compound.

The hindered hydrocarbyloxyamines are typically present from about 0.1% to about 1% by weight, the hydroxylamines, benzofuranones and/or amine oxides are typically present from about 0.01% to about 0.5% by weight, and the organic phosphorus compounds are typically present from 0.05% to about 0.5% by weight, based on the overall formulation.

The formulations are prepared by dry blending the appropriate additives with the polymer in a Turbula® blender for twenty minutes followed by melt compounding on a single screw extruder at 500° F. (260° C.) using a polyolefin screw fitted with a Maddock mixing section. Each formulation also contains 750 ppm calcium stearate, 250 ppm of the dialkylperoxide 2,5-bis(tert-butylperoxyl)-2,5-dimethylhexane (90% tech. grade) and 2200 ppm of the Clarifier-1 (Millad® 3988). Each 2 kg batch is split into 1 kg lots, where 1 kg is multiple pass extruded and the other is injection molded into Type IV tensile bars. The Type IV tensile bars, and a set of 125 mil plaques are split into three sets and treated with gamma irradiation from a ⁶⁰Co radiation source at 0, 30 and 60 Kilograys (or 0, 3 and 6 megarads) of exposure. The tensile bars are evaluated for retention of tensile strength and % elongation (at yield, at break) as a function of irradiation dose. The plaques are evaluated for changes in transparency or discoloration as a function of irradiation dose. The irradiated tensile bars, as well as the 125 mil plaques are then oven aged at 60° C. Color and haze development are measured weekly up to 4 weeks on the 125 mil plaques.

A typical organophosphorus stabilizer employed is tris(2,4-di-tert-butylphenyl) phosphite, bis(2,4-di-tert-butylphenyl) pentaerythritol diphosphite, tetrakis (2,4-di-tert-butylphenyl) 4,4′-biphenylene-diphosphonite or 2,4,6-tri-t-butylphenyl-(2-ethyl-2-propylpropylidene) phosphite. The amine oxide may be Genox™ EP, a di(C₁₆-C₁₈)alkyl methyl amine oxide, CAS# 204933-93-7. The hydroxylamine stabilizer is for example the N,N-di(alkyl)hydroxylamine produced by the direct oxidation of N,N-di(hydrogenated tallow)amine. The benzofuranone stabilizer may be 3-(3,4-dimethylphenyl)-5,7-di-tert-butyl-benzofuran-2-one.

The formulations including the present compounds show superior physical property and color retention.

### EXAMPLE 154

Solution phase Ziegler/Natta high density polyethylene copolymer (d=0.945 g/cm³) with a nominal melt flow rate of ca. 17 dg/min (2.16 kg @ 190° C.) samples are prepared with the additives by adding a 5% additive concentrate to the “additive free” pelleted base resin in a Turbula® blender for twenty minutes followed by melt compounding on a single screw extruder at 450° F. (232° C.) using a polyolefin screw fitted with a Maddock mixing section. The formulations contain the same additives at the levels described in Example 153. Each formulation contains 500 ppm of calcium stearate as an acid scavenger. Each 2 kg batch is split into 1 kg lots and 1 kg is multiple pass extruded and the other is injection molded into Type IV tensile bars or compression molded into 125 mil plaques.

The Type IV tensile bars, 125 mil plaques and 1^{st} pass extrusion pellets are split into three sets and treated with gamma irradiation from a ⁶⁰Co radiation source at 0, 30 and 60 Kilograys (or 0, 3 and 6 megarads) of exposure. The tensile bars are evaluated for retention of tensile strength and % elongation (at yield, at break), the plaques are evaluated for discoloration, and the pellets are tested for retention of melt flow rate, all as a function of irradiation dose. The irradiated tensile bars, as well as the 125 mil plaques, are oven aged at 60° C. Color development, tensile strength and % elongation are measured during oven aging at 60° C.

The formulations containing the hindered hydrocarbyloxyamines show superior physical property and color retention.

### EXAMPLE 155

Polypropylene homopolymer, Ti/Al catalyst, bulk phase process, with a nominal melt flow index of ca. 15 dg/min at 2.16 kg/230° C. is extruded into fibers at 525° F. and a draw ratio of 3.5:1 and 15 Denier per filament. The fibers are knitted into socks. Samples are also compression molded into plaques. The individual formulations each contain a 1:1 blend of calcium stearate/dihydrotalcite at a total level of 500 ppm as an acid scavenger. Formulations are otherwise prepared as per Example 153.

The fibers, socks and plaques are treated with gamma irradiation from a ⁶⁰Co radiation source at 0, 30, and 60 Kilograys (or 0, 3 and 6 megarads) of exposure.

The formulations containing the present hindered hydrocarbyloxyamines show superior color and/or physical property retention. Such formulations are suitable for woven or nonwoven fibers or filaments or fabrics prepared therefrom.

### EXAMPLE 156

Unipol®, Union Carbide Corporation, gas phase E/H LLDPE copolymer; Ti/Al catalyst; melt index ca. 1 dg/min. at 2.16 kg/190° C. is extruded into blown films at 450° F. to produce 1.5 mil films. The individual formulations each contain zinc stearate at a total level of 500 ppm as an acid scavenger. Formulations are otherwise prepared as per Example 153.

The films are treated with gamma irradiation from a ⁶⁰Co radiation source at 0, 30 and 60 Kilograys (or 0, 3 and 6 megarads) of exposure.

The films containing the formulations of the present invention show superior physical property and color retention.

### EXAMPLE 157

Forming spunbonded fabrics is a conventional process well known in the art. Fiber grade polypropylene is dry blended with 10% loading of the test additive and then melt compounded at 220° C. into masterbatch pellets. The master batch pellets are dry blended with polypropylene resin (MFR=35-50) at a ratio to yield 1.0% additive. Spunbonded fibers are prepared by extrusion of molten polypropylene resin (die temperature=230° C.) as filaments from a plurality of fine circular capillaries of a spinneret. Cooling air is fed into a quenching chamber (2,400 rpm) wherein the filaments are cooled. The cooling air is then sucked through a nozzle, which accelerates the flow of air creating a force that draws the filaments. The drawn filaments are then passed through a diffusor and deposited on a conveyor belt (33 m/min) to form a non-woven fabric.

Forming meltblown fabrics is a conventional process well known in the art. Polypropylene samples are dry blended with 10% loading of a test additive of formulae (1)-(45) respectively, and then melt compounded at 220° C. into masterbatch pellets. The master batch pellets are dry blended with polypropylene resin (MFR 1200) at a ratio to yield 1.0% additive. Meltblown fibers are prepared by extrusion of molten polypropylene resin as filaments from a plurality of fine circular capillaries of a spinneret. A high-velocity heated air stream attenuates the filaments of molten polypropylene to reduce their diameter. There after the meltblown fibers are carried by the high-velocity heated air stream and are deposited on a collection surface to form a web of randomly dispersed meltblown fibers. Thermal bonding of the web to retain integrity and strength occurs as a separate downstream operation.

The nonwoven fabrics containing the present compounds show good UV stability in agricultural applications such as direct covers, small tunnel covers, and shade cloths and also show good stability after exposure to agricultural chemicals such as pesticides and herbicides.

### EXAMPLE 158

### Rotomolding

100 parts medium density polyethylene, copolymerized with hexene (Novapol® TR-0735, nominal melt index 6.8 g/10 min., density 0.935 g/cm³) are dry blended with 0.050 parts of calcium stearate and a combination of additional stabilizers (see below). The mixtures are melt compounded into pellets at 232° C. in a Superior/MPM extruder using a 24:1 L/D screw with Maddock mixing head at 100 rpm.

The compounded pellets are ground to a uniform particle size (150-500 μm) prior to the rotational molding process. This grinding step increases the surface area of the particles leading to a faster heat absorption, and thus reducing overall energy consumption.

The rotational molding process is performed in a laboratory scale equipment FSP M20 “Clamshell”. The ground resin is placed in a cast aluminum mold, which is rotated biaxially in a gas-fired oven. Hot air is circulated by blowers in the chamber while the temperature is increased to 288° C. within 4 minutes. This temperature is maintained for a specific time (see Tables below). Subsequently, the oven is opened and while still rotating, the mold is cooled with forced air circulation for 7 minutes, followed by water spray mist for 7 minutes, and an additional air cooling step for 2 minutes. Throughout the entire heating and cooling cycles, the speed of the major axis is maintained at 6 rpm with a 4.5:1 ratio of rotation. After the cooling cycles, the mold is opened and the hollow object removed.

Formulations are additionally blended with a combination of 0.100 parts of a phosphite process stabilizer, 0.050 parts of a hydroxylamine process stabilizer and 0.200 parts of the instant compounds.

Formulations are additionally blended with a combination of 0.100 parts of a phosphonite process stabilizer, 0.050 parts of a hydroxylamine process stabilizer and 0.200 parts of the instant compounds.

The phosphite stabilizer is tris-(2,4-di-tert-butylphenyl)phosphite or bis(2,4-di-tert-butylphenyl) pentaerythritol diphosphite. The phosphonite stabilizer is tetrakis(2,4-di-tert-butylphenyl)4,4′-biphenylene-diphosphonite. The hydroxylamine stabilizer is the N,N-di(alkyl)hydroxylamine produced by the direct oxidation of N,N-di(hydrogenated tallow)amine.

The formulations are rotationally molded into hollow objects according to the general procedure with hold times of 6 to 14 minutes.

Yellowness Index of the outer surface is determined on a DCI SF600 spectrophotometer according to ASTM D 1925. An increase in yellowness corresponds to a positive increase in the Yellowness Index. The formulations containing the instant compounds exhibit excellent color stability at these processing conditions.

Low-temperature impact strength testing is performed with an instrumented drop weight (11.34 kg/50.8 cm) impact apparatus Dynatup® 8250. Test specimens are conditioned in an air circulated freezer for no less than 12 hours at −40° C. prior to test.

The impact strength results are reported in % brittleness. Formulations containing the instant compounds exhibit excellent stability with regard to the failure mode based on low-temperature impact strength testing at these processing conditions.

Formulations containing the instant compounds also exhibit excellent gas fading resistance. The stabilizer systems described above also exhibit reduced cycle times in the polyolefin rotational molding process compared to the current state of the art systems.

The phosphite bis(2,4-di-tert-butyl-6-methylphenyl) ethyl phosphite may be substituted for the organic phosphorus compounds in the above formulations with excellent results.

Hollow articles are also prepared replacing the hydroxylamine stabilizer used in the formulations with the amine oxide Genox™ EP. Genox™ EP is a di(C₁₆-C₁₈)alkyl methyl amine oxide, CAS# 204933-93-7, available from GE Specialty Chemicals. These formulations also exhibit excellent color stability, mechanical stability and improved cycle times.

### EXAMPLE 159

Articles, films and fibers prepared according to the present examples, which are fabricated using recycled plastics as part of the formulation and the stabilizers of the present invention, exhibit stability against the deleterious effects of actinic light and thermal exposure.

### EXAMPLE 160

All additives are commercial materials. Titanium dioxide is DuPont Ti-PURE® R-104. Polycarbonate (PC) is LEXAN®141-111 natural; ABS is Dow MAGNUM® 342EZ. Polymers and additives are extrusion compounded in one pass using a twin screw extruder of screw design 18 mm, co-rotating, non-intermeshing; processing temperature 240° C., with a die melt temperature 260° C. Injection molding of Izod bars (2.5″L×0.5″W×0.125″W) is done on a BOY 30 machine, barrel temperature 475-515° F., die temperature 515° F.

Accelerated weathering is performed using an Atlas Ci65A Weather-Ometer (“XAW”), operated in either “Dry XAW” mode (ASTM G26-90 method C) or interior auto (“IAXAW”) (black panel temperature 89° C., light/dark cycle 3.8 hr/1.0 hr; irradiance 0.55 watt/m², inner filter-quartz, outer filter-high borate.

Yellowness Index and delta E color are performed as per ASTM D-1925, using a Chroma-Sensor CS-5 Colorimeter, operated at 10 degree, small area view, specular included.

Chip impact is performed per ASTM D4508-90 on a TMI Monitor/Impact Tester using a 30 ft-lb weight. Per ASTM D4508-90 paragraph 4.6, the impact strength results are reported as a complete break, a partial break or a non-break, with a non-break specimen exhibiting the most desired impact retention, and partial break being the next desired impact retention. Data presented are the average of ten replicate samples for each formulation.

Melt rheology is performed using a Kayeness Galaxy V capillary rheometer, equipped with a 1000 pound load cell, a die of orifice radius 0.015 inch and orifice length 1.0000 inch., operated at the stated test temperature. Samples are pre-dried in a vacuum oven under nitrogen atmosphere to an analyzed moisture level of less than 100 ppm prior to rheology testing.

The stabilizers used in the tests below are:

UVA: 2-(2-hydroxy-3,5-di-alplha-cumylphenyl)-2H-benzotriazole or 4,6-diphenyl-2-(4-hexyloxy-2-hydroxyphenyl)-s-triazine

Phosphite is tris(2,4-di-tert-butylphenyl) phosphite.

Chip impact bars are prepared and subjected to dry XAW accelerated weathering. The samples are removed at selected intervals for color measurement and then for destructive chip impact testing.

Formulations containing 0.75 weight percent of the present hindered amines and 0.5 weight percent of UVA exhibit outstanding color stability and retention of useful mechanical properties as measured by chip impact strength.

All formulations contain 0.1% Phosphite; all are 50/50 wt/wt PC and ABS.

### EXAMPLE 161

Chip impact bars are prepared as per Example 160 and subjected to accelerated weathering under interior auto Xenon Arc Weather-Ometer (IAXAW) conditions. Test samples are removed at intervals for color measurement and then destructive chip impact testing. The IAXAW conditions are more severe than dry Xenon (XAW) conditions due to higher temperature and higher irradiance energy.

Formulations containing 0.75 weight percent of the present hindered amines in addition to 0.5 weight percent of UVA exhibit outstanding color stability and retention of useful mechanical properties as measured by chip impact strength.

All formulations contain (0.1%) tris(2,4-di-tert-butylphenyl) phosphite; all are 50/50 wt/wt PC and ABS.

### EXAMPLE 162

Formulations of a 50/50 wt/wt blend of PC and ABS which contain 1.0 wt. % titanium dioxide (TiO₂), 0.75 weight percent of the present hindered amines and 0.50 weight percent of either 2-(2-hydroxy-3,5-di-alplha-cumylphenyl)-2H-benzotriazole or 4,6-diphenyl-2-(4-hexyloxy-2-hydroxyphenyl)-s-triazine are prepared and tested as per Example 160. The formulations exhibit outstanding resistance to color change (delta E). The chip impact values are favorably high as well.

All formulations contain (0.1%) tris(2,4-di-tert-butylphenyl) phosphite; all are 50/50 wt/wt PC and ABS.

### EXAMPLE 163

The retention of molecular weight during processing of a polymer or polymer blend is critical for the successful production of a useful article. Additives that interact adversely with the polymer substrate during processing are of limited or no value to the production of a final useful article. The adverse interactions may be due to various factors, such as a chemical reaction between the additive and the polymer that results in a reduction of polymer molecular weight by chain cleavage. Also, if the additive itself degrades during the required high temperature processing of the polymer, then the degradation products of the additive itself may interact antagonistically with the polymer.

An acceptable hindered amine stabilizer for use in a polycarbonate blend should exhibit minimal deleterious interaction with the polymers during their high temperature processing into useful articles. Hindered amines that are basic (having a pKa value >7 typically) are known to cause degradation of polycarbonates, especially during melt processing of the polymer as taught by G. L. Gaines, Jr. (Polymer Degradation and Stability, 27 13-18 (1990)). Melt rheology testing is an accepted method to assess the stability of a polymer in the melt state (see ASTM D 3835-90) and thus to relate the interaction of additives to changes in molecular weight of the polymers. Polymer molecular weight, and changes in molecular weight, may be expressed in terms of the polymer apparent melt viscosity. A decrease in apparent melt viscosity over time of the test indicates that polymer degradation and molecular weight reduction are occurring whereas an increase in apparent melt viscosity over time indicates polymer degradation may be occurring by a crosslinking or a molecular weight buildup mechanism. It is often convenient to represent this change in melt viscosity as the melt viscosity ratio as taught by A. B. Auerbach et al., Polymer Engineering and Science, 30, 1041-1050 (1990). The melt viscosity ratio (MVR) may be defined as the change in melt viscosity (η) over time, and expressed as the ratio of a melt viscosity at some extended test time (ηₓ), divided by the initial melt viscosity (ηₒ) ultimately expressed as (ηₓ/ηₒ).

Formulations of 50/50 wt/wt PC/ABS samples containing the present hindered amines, alone and polymer samples additionally containing various additives that are incorporated as described for Examples 160 and 161 exhibit low detrimental impact on apparent melt viscosity of formulations.

The PC/ABS blends of Examples 160-62 may be replaced with PC/ASA blends which also exhibit excellent color stability and mechanical stability as measured by impact strength and melt flow stability.

The PC/ABS blends of Examples 160-62 may be replaced with PC/polyester blends such as PC/PET and PC/PBT blends which also exhibit excellent color stability and mechanical stability as measured by impact strength and melt flow stability.

ASA is acrylonitrile-styrene-acrylate, the acrylate is typically butyl acrylate. PET is polyethylene terephthalate. PBT is polybutylene terephthalate. ABS is acrylonitrile-butadiene-styrene.

### EXAMPLE 164

### Coextrusion over PVC

A sheet composition suitable for use in weatherable house siding is prepared by coextrusion of a 0.010″ thick light stabilized rigid PVC layer (“cap layer”) over a 0.060″ thick rigid PVC bulk substrate (“bulk layer”). Composition of the layers is given below.

| | |
|---|---|
| Cap | 100.00 phr PVC |
| Layer | 2.50 phr IRGASTAB ® T 634 (thermal stabilizer) |
| | 6.00 phr KM-334 (acrylic impact modifier -Rohm & Haas) |
| | 1.50 phr K-120N (processing aid - Rohm & Haas) |
| | 0.60 phr Paraffin Wax 165 (Rheolube) |
| | 0.30 phr PE Wax AC629A (Honeywell) |
| | 0.50 phr Gray color concentrate |
| | 4.70 phr R-960 titanium dioxide (DuPont) |
| | 0.50 phr Instant Compound |
| | 0.50 phr BZT UV absorber |
| Bulk | 100.00 phr PVC |
| Layer | 2.50 phr IRGASTAB ® T 634 (thermal stabilizer) |
| | 6.00 phr KM-334 (acrylic impact modifier - Rohm & Haas) |
| | 1.50 phr K-120N (processing aid - Rohm & Haas) |
| | 0.60 phr Paraffin Wax 165 (Rheolube) |
| | 0.30 phr PE Wax AC629A (Honeywell) |

The weatherability of the sheets of the present invention, containing the instant hindered amines, respectively, is superior to a control sheet prepared without the use of NOR HALS in the cap layer.

BZT UV absorber is 2-(3,5-di-t-amyl-2-hydroxyphenyl)-2H-benzotriazole.

The PVC cap layer may be replaced with ASA, PMMA, polyvinylidene fluoride (PVDF) or polypropylene-PMMA graft copolymer (PP-g-PMMA) cap layers as per the following formulations. In each case the bulk layer is as above.

| | |
|---|---|
| Cap Layer | 100.00 phr ASA (e.g. GELOY ® from GE Plastics) |
| | 0.50 phr Gray color concentrate |
| | 4.70 phr R-960 titanium dioxide (DuPont) |
| | 0.50 phr Instant Compound |
| | 0.50 phr BZT UV absorber A |
| | 0.10 phr process stabilizer A |
| Cap Layer | 100.00 phr impact-modified PMMA |
| | 0.50 phr Gray color concentrate |
| | 4.70 phr R-960 titanium dioxide (DuPont) |
| | 0.50 phr Instant Compound |
| | 0.50 phr BZT UV absorber B |
| | 0.10 phr process stabilizer A |
| Cap Layer | 100.00 phr PVDF (e.g. KYNAR ® from Elf Atochem) |
| | 0.50 phr Gray color concentrate |
| | 4.70 phr R-960 titanium dioxide (DuPont) |
| | 0.50 phr Instant Compound |
| | 0.50 phr BZT UV absorber C |
| Cap Layer | 100.00 phr PP-g-PMMA (e.g. INTERLOY ® from Montell) |
| | 0.50 phr Gray color concentrate |
| | 4.70 phr R-960 titanium dioxide (DuPont) |
| | 0.20 phr Instant Compound |
| | 0.20 phr oligomeric HALS |
| | 0.20 phr BZT UV absorber A |
| | 0.10 phr process stabilizer B |

In each case, the weatherability of the sheets of the present invention, containing the instant hindered amines is superior to a control sheet prepared without the use of NOR HALS in the cap layer.

BZT UV absorber A is 2-(3,5-di-t-amyl-2-hydroxyphenyl)-2H-benzotriazole; BZT UV absorber B is 2-(2-hydroxy-3,5-di-alpha-cumylphenyl)-2H-benzotriazole; BZT UV absorber C is 2-(3-t-butyl-2-hydroxy-5-(2-(ω-hydroxy-octa-(ethyleneoxy)carbonyl-ethyl)-, phenyl)-2H-benzotriazole. Oligomeric HALS is N-{[2-(N-2,2,6,6-tetramethylpiperidin-4-yl)butylamino]-s-triazin-4-yl}-N,N′-bis(2,2,6,6-tetramethylpiperidin-4-yl)-1,6-hexanediamine terminated with 2,4-bis(dibutylamino)-s-triazin-6-yl. Process stabilizer A is a 80:20 blend of tris(2,4-di-tert-butylphenyl) phosphite and β-(3,5-di-tert-butyl-4-hydroxyphenyl)propionic acid octadecanoic ester. Process stabilizer B is a 1:1 blend of tris(2,4-di-tert-butylphenyl) phosphite and the N,N-di(alkyl)hydroxylamine produced by the direct oxidation of N,N-di(hydrogenated tallow)amine.

### EXAMPLE 165

### Coextrusion over Polycarbonate

A sheet composition suitable for use in weatherable glazing is prepared by coextrusion of a 0.010″ thick light stabilized PMMA layer (“cap layer”) over a 0.100″ thick polycarbonate bulk substrate (“bulk layer”). Composition of the layers is given in the table below.

| | |
|---|---|
| Cap Layer | 100.00 phr PMMA |
| | 0.10 phr process stabilizer |
| | 0.25 phr Instant Compound |
| | 3.50 phr HPT UV absorber |
| Bulk Layer | 100.00 phr Polycarbonate (e.g. LEXAN ® 141 from GE) |
| | 0.08 phr phosphite process stabilizer |
| | 0.10 phr BZT UV absorber |

The weatherability of the sheets of the present invention, containing the instant hindered amines is superior to a control sheet prepared without the use of NOR HALS in the cap layer.

Coextruded sheets are also prepared, replacing HPT UV absorber in the cap layer with each of 2,2′-methylene-bis(4-t-octyl-(6-2H-benzotriazol-2-yl)phenol) and 2-(2-hydroxy-3,5-di-alpha-cumylphenyl)-2H-benzotriazole. Excellent results are achieved for these coextruded sheets containing the instant hindered amines.

BZT UV absorber is 2-(2-hydroxy-3,5-di-alpha-cumylphenyl)-2H-benzotriazole, HPT UV absorber is 4,6-diphenyl-2-(4-hexyloxy-2-hydroxyphenyl)-s-triazine. Phosphite stabilizer is tris(2,4-di-tert-butylphenyl) phosphite. Process stabilizer is a 80:20 blend of tris(2,4-di-tert-butylphenyl) phosphite and β-(3,5-di-tert-butyl-4-hydroxyphenyl)propionic acid octadecanoic ester.

### EXAMPLE 166

### Coextrusion

A composition suitable for use as weatherable window profile is prepared by coextrusion of a 0.010″ thick light stabilized ASA layer (“cap layer”) over a 0.060″ thick ABS bulk substrate (“bulk layer”). Composition of the layers is given in the table below.

| | |
|---|---|
| Cap Layer | 100.00 phr ASA (e.g. GELOY ® from GE Plastics) |
| | 4.00 phr R-960 titanium dioxide (DuPont) |
| | 0.50 phr Instant Compound |
| | 0.50 phr BZT UV absorber |
| | 0.10 phr process stabilizer |
| Bulk Layer | 100.00 phr ABS (e.g. CYCOLAC ® from GE Plastics) |
| | 0.10 phr process stabilizer |

The weatherability of the sheets of the present invention, containing the instant hindered amines, is superior to a control sheet prepared without the use of NOR HALS in the cap layer.

BZT UV absorber is 2-(2-hydroxy-3,5-di-alpha-cumylphenyl)-2H-benzotriazole. Process stabilizer is a 80:20 blend of tris(2,4-di-tert-butylphenyl) phosphite and β-(3,5-di-tert-butyl-4-hydroxyphenyl)propionic acid octadecanoic ester.

### EXAMPLE 167

### Multilayer Polymer Structures

The instant compounds have utility as light stabilizers to protect light sensitive polymers present in multilayer polymer structures. Examples of such polymer structures include but are not limited to:

1.) Sheets and signs as seen in WO97/42261; and U.S. Pat. No. 5,387,458 which are incorporated herein by reference;

2.) Solar Control Films of Various Construction as seen in U.S. Pat. Nos. 3,290,203, 3,681,179, 3,776,805 and 4,095,013, incorporated herein by reference; and

3.) Base stock or cap stock for coextrusion structures such as window profiles, laminates over automotive bumpers or auto exterior panels.

Window profiles include photosensitive polymers such as ABS, ASA, SAN or vinylogous polymers such as PVC. Automotive polymeric materials which are photosensitive include for example ABS, SAN, ASA and polycarbonate as well as blends such as PC/ABS, which include Pulse® from Dow, Cycoloy® from GE, Bayblend® from Bayer, PC/PBT known as Xenoy® from GE, PC/ASA such as Geloy® from GE, and the “W-4” polymer as disclosed by General Electric Company (Modern Plastics May 2000 pages 90-91).

The instant hindered amines of this disclosure act to protect against photolytic degradation of a polymer component, or an incorporated pigment, dye colorant, or protect adhesive or “tie-layers” in such constructions.

A multilayer polymer composite is prepared by different routes, such as co-extrusion of one or more polymer compositions to form the multilayer composite. Alternatively, compression molding or thermoforming of one or polymer compositions produces the desired polymer composite. In particular, these techniques are used in the manufacture of signage, typically composed of one or more layers of polymeric materials formed on top of a base material (metal sheet, plastic, etc).

Examples of potential polymeric materials that may comprise one or more sections of the laminate, sign, sheet or composite structure may include:

polycarbonate

polyesters such as PET, PBT, PEN, PTT

acrylics such as PMMA and acrylate copolymer or terpolymers

polyolefins

vinylogous polymers and copolymers composed of vinyl chloride, vinyl acetate,

vinylidene chloride, vinylidene fluoride.

The present hindered amines each provide excellent stabilization to such constructions.

### EXAMPLE 168

### Photo-Cured White Screen Ink

A model white UV-curable screen ink, based on urethane acrylate chemistry, is prepared with a fixed pigment to binder ratio of 0.74. A base formulation is prepared which is complete excluding a photoinitiator and a hindered amine of component (b) and consists of:

Rutile TiO₂, 500.0 g

Ebecryl® 284, 300.0 g

Ebecryl® 810, 100.0 g

Trimethylolpropane ethoxy triacrylate (TMPEOTA), 60.0 g

Tripropylene glycol diacrylate (TRPGDA), 180.0 g

Isobornyl acrylate (IBOA), 30.0 g

Modaflow® 2100, 5.0 g

Byk®-A 501, 5.0 g

Aerosil® 200, 20.0 g

The TiO₂ is added as a 66% dispersion in a portion of the Ebecryl® 284. The Aerosil® 200 is added as a 10% dispersion in the TRPGDA.

Ebecryl® 284 is an acrylated aliphatic urethane oligomer/monomer blend. Ebecryl® 810 is a polyester acrylate oligomer. The Ebecryl® products as well as the acrylate monomers are available from UCB Chemicals Corp., Smyrna, Ga. Modaflow® 2100 is an acrylic flow agent available from Monsanto. Byk®-A 501 is an air release agent available from Byk-Chemie. Aerosil® 200 is a fumed silica viscosity modifier from Degussa.

To a portion of the base formulation is added a photoinitiator mixture of bis(2,4,6-trimethylbenzoyl)phenylphosphine oxide/1-hydroxycyclohexylphenylketone in a 35/65 ratio. The photoinitiator mixture is 4.78 weight percent of the formulation.

The formulations further contain 1% each of the instant hindered amines of the present disclosure.

Eight replicate prints of each formulation are prepared. Application is made via a 355 mesh screen and applied on polycarbonate sheets. Samples are cured with a moving belt at 50 feet/min under two medium pressure mercury lamps perpendicular to the belts @ 200 watts/in. each. All prints receive two passes under the lamps. Final layer thickness is approximately 1.2 to 1.3 mils (31-34 microns).

The present hindered amines provide excellent stabilization to the photo-cured inks.

### EXAMPLE 169

### Photo-Cured White Pigmented Coating

A model white UV-curable coating for wood is prepared based on polyester acrylate chemistry with a titanium dioxide level of 25% by weight. A base formulation is prepared which is complete excluding a photoinitiator hindered amine of component (b):

Rutile TiO₂, 100.0 g

Ebercryl® 830, 240.0 g

HDODA, 42 g

TMPTA, 18.0 g

The TiO₂ is added as a 63% dispersion in a portion of the Ebercryl® 830. Ebercryl® 830 is a hexafunctional polyester acrylate oligomer. HDODA is 1,6-hexanediol diacrylate. TMPTA is trimethylolpropane triacrylate. The Ebecryl® products as well as the acrylate monomers are available from UCB Chemicals Corp., Smyrna, Ga.

To a portion of the base formulation is added a photoinitiator mixture of bis(2,4,6-trimethylbenzoyl)phenylphosphine oxide/1-hydroxycyclohexylphenylketone in a 1:2 ratio. The photoinitiator mixture is 3.0 weight percent of the total formulation.

The formulations further contain 1% each of the instant hindered amines of the present disclosure.

Eight replicate prints of each formulation are prepared. Films are prepared with a draw-down bar over a white Scotchcal® vinyl film from 3M. Samples are cured with a moving belt at 58 feet/min. under two medium pressure mercury lamps perpendicular to the belts @ 300 watts/in. each. The prints received one pass under the lamps. Irradiance received is 618 mJ/cm². Final cured thickness is 2.1 mils (53 microns).

The present hindered amines provide excellent stabilization to the photo-cured coatings.

### EXAMPLE 170

### Photo-Cured Clear Coating

A model clear UV-curable coating for wood is prepared based on acrylated aromatic urethane/epoxy chemistry. A base formulation is prepared which is complete excluding a photoinitiator and hindered amines of component (b):

Ebercryl® 4827, 30.0 g

Ebercryl® 600, 30.0 g

Tripropylene glycol diacrylate (TRPGDA), 40.0 g

Ebercryl® 4827 is an aromatic urethane diacrylate oligomer. Ebercryl® 600 is the diacrylate ester of a bisphenol-A epoxy resin. The Ebecryl® products as well as the acrylate monomers are available from UCB Chemicals Corp., Smyrna, Ga.

To a portion of the base formulation is added a photoinitiator mixture of bis(2,4,6-trimethylbenzoyl)phenylphosphine oxide/1-hydroxycyclohexylphenylketone in a 1:2 ratio. The photoinitiator mixture is 3.0 weight percent of the formulation.

The formulations further contain 1% each of the instant hindered amines of the present disclosure.

Four replicate prints of each formulation are prepared. Films are prepared with a draw-down bar over a white Scotchcal® vinyl film from 3M. Samples are cured with a moving belt at 95 feet/min. under two medium pressure mercury lamps perpendicular to the belts @ 300 watts/in. each. The prints received two passes under the lamps. Irradiance received is 750 mJ/cm². Final cured thickness is 5.1 mils (130 microns).

The coatings containing in addition to a bisacylphosphine oxide photoinitiator, a hindered amine of the instant invention display excellent stability.

### EXAMPLE 171

### Photo-Cured White Powder Coating

A typical white, radiation-curable powder coating resin composition consists of the following components in parts by weight: 5-6 parts of an unsaturated polyester amorphous oligomer, 1 part divinyl ether crystalline monomer, 2-3.5 parts rutile titanium dioxide, 0.015 parts flow-aid, 0.02 parts bis(2,4,6-trimethylbenzoyl)phenylphosphine oxide and 0.004 parts 1-(4-(2-hydroxyethoxy)-phenyl)-2-hydroxy-2-methyl-propan-1-one. The ingredients are blended together in an extruder and ground into a fine powder. The powder is applied to the substrate to be coated and is subsequently melted with an infrared heat source which allows for continuous film formation. In the melt state the resin is exposed to the radiation source to initiate curing.

The formulations further contain 1% each of the instant hindered amines of the present disclosure.

The white powder coatings containing a hindered amine of the present invention display excellent stability.

### EXAMPLE 172

### Photo-Cured White Gel Coat

A typical gel coat formulation consists of an unsaturated polyester oligomer with a styrene diluent added to control viscosity. Styrene is normally present at about 35% by weight. The other components are typically rutile TiO₂, about 10% by weight and bis(2,4,6-trimethylbenzoyl)phenylphosphine oxide, about 2% by weight. The mixture is either sprayed, brushed or drawn down on the substrate and cured to a glassy solid state. The cure line speeds are about 60 feet per minute per lamp with Fusion D lamps and about 24 feet per minute per lamp with standard mercury lamps. The film thickness is about 20 mils.

The formulations further contain 1% each of the instant hindered amines of the present disclosure.

The white gel coats containing a hindered amine of the instant invention display excellent stability.

### EXAMPLE 173

Molded test specimens are prepared by injection molding polypropylene pellets containing a phosphite, a phenolic antioxidant or hydroxylamine, a metal stearate, ultraviolet light absorbers or hindered amine stabilizers or a mixture of UV absorbers and hindered amine stabilizers and, optionally, pigment.

Polypropylene pellets are prepared from stabilizers, co-additives and commercially available polypropylene by mixing the components in a Superior/MPM 1″ single screw extruder with a general all-purpose screw (24:1 L/D) at 475° F. (250° C.), cooled in a water bath and pelletized. The resulting pellets are molded into 60 mil (0.06 inch thick) 2″×2″ plaques at about 475° F. (250° C.) on a BOY 30M Injection Molding Machine. The formulations may contain, optionally, pure pigment or pigment concentrates.

Polypropylene formulations composed of polypropylene homopolymer or polypropylene copolymer are stabilized with a base stabilization system consisting of an N,N-dialkylhydroxylamine or a hindered phenolic antioxidant with or without an organophosphorous compound.

All additive and pigment concentrations in the final formulations are expressed as weight percent based on the resin.

Formulations contain polypropylene pellets, hindered hydrocarbyloxyamines of the instant invention from about 0.1% to about 1%, and one or more of the following components;

0.0%-5.0% pigment,

0.0%-50.0% glass fiber,

0.0%-0.5% phosphite,

0.0%-1.25% phenolic antioxidant,

0.0%-0.5% hydroxylamine,

0.05%-0.10% calcium stearate,

0.0%-1.25% UV absorber,

0.0%-30% poly(propylene-g-maleic anhydride)

The components are dry blended in a tumble dryer prior to extrusion and molding.

Test plaques are mounted in metal frames and exposed in an Atlas Ci65 Xenon Arc Weather-o-meter at 70° C. black panel temperature, 0.55 W/m² at 340 nanometers and 50% relative humidity with intermittent light/dark cycles and water spray (Society of Automotive Engineers—SAE J 1960 Test Procedure). Specimens are tested at approximately 625 kilojoule intervals by performing color measurements on an Applied Color Systems spectrophotometer by reflectance mode according to ASTM D 2244-79. Data collected included delta E, L*, a* and b* values. Gloss measurements are conducted on a BYK-GARDNER Haze/Gloss Meter at 60° according to ASTM D523.

The formulations that include an instant compound show superior light stability as measured by color retention, gloss retention and resistance to surface chalking.

### EXAMPLE 174

Formulations prepared as in Example 173 with thermoplastic olefin in place of polypropylene exhibit superior retention of gloss, color and appearance. Thermoplastic olefin examples include polypropylene homopolymer and polypropylene copolymer blends with EPDM, EPR and poly(ethylene-alpha-olefin) co-polymers.

What is claimed is:

1. A water compatible or water soluble sterically hindered alkoxyamine or hydroxy substituted alkoxyamine compound selected from the group consisting of compounds of formulae (1)-(10) 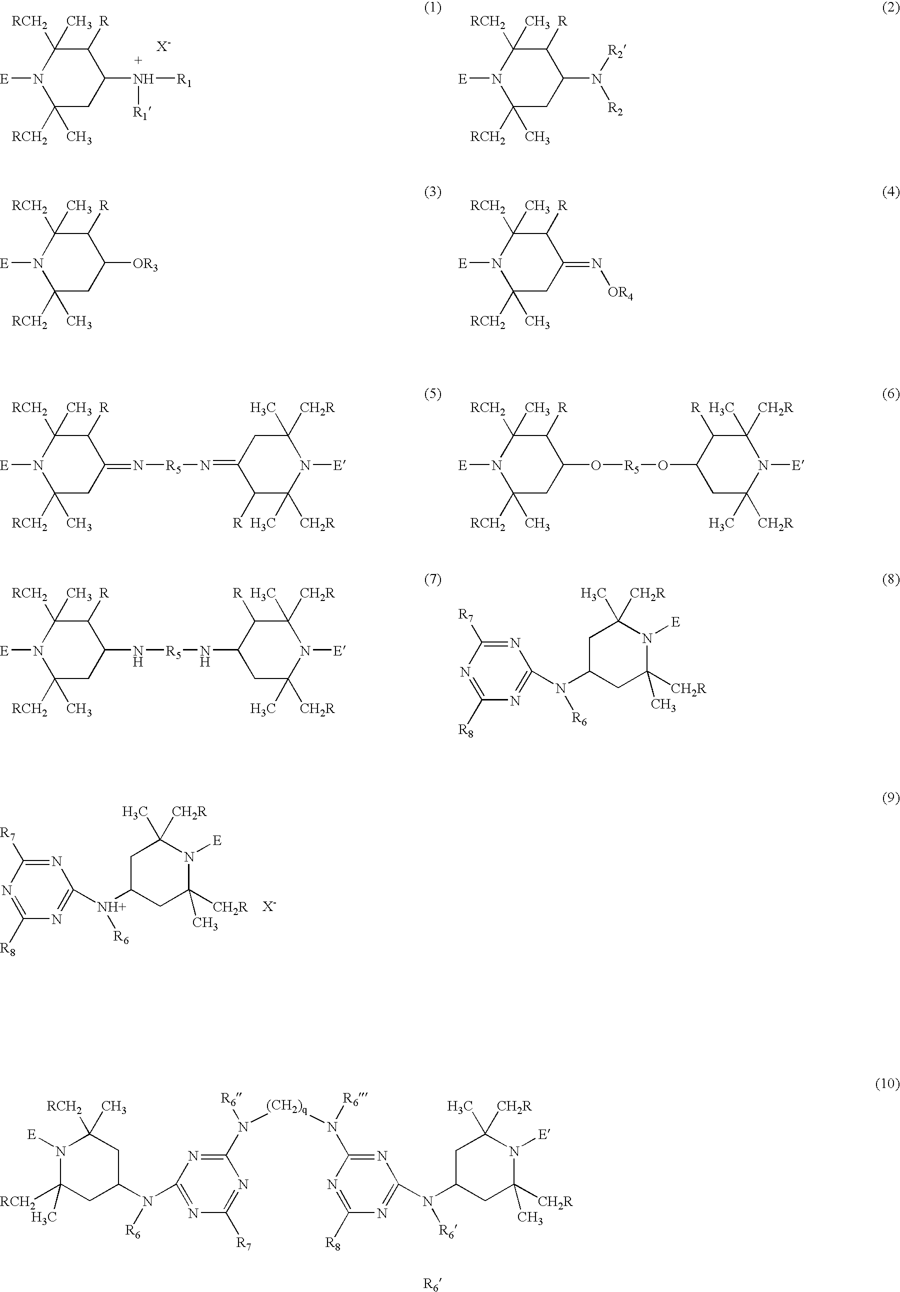
where
E is alkoxy of 1 to 18 carbon atoms, cycloalkoxy of 5 to 12 carbon atoms or aralkoxy of 7 to 15 carbon atoms, or E is -O-T-(OH)_{b},
T is a straight or branched chain alkylene of 1 to 18 carbon atoms, cycloalkylene of 5 to 18 carbon atoms, cycloalkenylene of 5 to 18 carbon atoms, a straight or branched chain alkylene of 1 to 4 carbon atoms substituted by phenyl or by phenyl substituted by one or two alkyl groups of 1 to 4 carbon atoms;
b is 1, 2 or 3 with the proviso that b cannot exceed the number of carbon atoms in T, and when b is 2 or 3, each hydroxyl group is attached to a different carbon atoms of T;
E′ is hydrogen, C₁-C₁₈alkyl, C₂-C₁₈alkenyl, C₇-C₁₅phenylalkyl, C₂-C₁₈alkanoyl or phenyl, or E′ is independently defined as for E,
R is hydrogen or methyl,
R₁ is hydrogen, C₁-C₁₂alkyl, C₅-C₈cycloalkyl, C₅-C₈cycloalkyl substituted by one to three C₁-C₄alkyl, C₂-C₁₂alkenyl, phenyl, C₇-C₉phenylalkyl, glycidyl, C₂-C₁₂alkanoyl, C₆-C₉cycloalkylcarbonyl, C₂-C₁₂carbamoyl, C₂-C₁₂alkenoyl, benzoyl, benzoyl substituted by one to three C₁-C₄alkyl, C₂-C₁₂alkanoyl substituted by a di(C₁-C₆alkyl) phosphonate,
or R₁ is C₂-C₁₂alkyl, C₂-C₁₂alkanoyl or C₇-C₁₈phenylalkyl, each interrupted by one to six oxygen, sulfur or —N(R₆)— groups; C₁-C₁₂alkyl, C₂-C₁₂alkanoyl, phenyl or C₇-C₁₈phenylalkyl, each substituted by one to six hydroxy groups or by one to six —NHR₆ groups; C₂-C₁₂alkyl, C₂-C₁₂alkanoyl or C₇-C₁₈phenylalkyl, each interrupted by one to three —NR₆C(O)— groups; or C₁-C₁₂alkyl, C₂-C₁₂alkanoyl, phenyl or C₇-C₁₈phenylalkyl, each substituted by one to three —SO₃H groups or by one to three —COOR₆ groups; or
R₁ is said alkyl substituted by a piperazine or by a morpholine group; or
R₁ is said interrupted group further substituted by one to six hydroxy groups or by one to six —NHR₆ groups; or
R₁ is said interrupted group further substituted by one to three —SO₃H groups or by one to three —COOR₆ groups;
or R₁ is a mono-valent homo- or co-oligomer consisting of monomer units derived from monomers selected from the group consisting of ethylene oxide, propylene oxide, ethylene glycol, propylene glycol, acrylic acid, methacrylic acid, ethylene imine, acrylamide, vinyl formamide, vinyl alcohol and vinyl acetate; which homo- or co-oligomer consists of between 2 and about 24 monomer units;
R₁′ is independently defined as for R₁,
R₅ is a divalent homo- or co-oligomer consisting of monomer units derived from monomers selected from the group consisting of ethylene oxide, propylene oxide, ethylene glycol, propylene glycol, acrylic acid, methacrylic acid, ethylene imine, acrylamide, vinyl formamide, vinyl alcohol and vinyl acetate; which homo- or co-oligomer consists of between 2 and about 24 monomer units,
R₆ is hydrogen or C₁-C₆alkyl,
R₆′, R₆″ and R₆″′ are independently defined as for R₆,
R₇ is —N(R₂)(R₂′) or is chlorine, alkoxy of 1 to 12 carbon atoms, 2-hydroxyethylamino or —N(R₆)(R₆′); 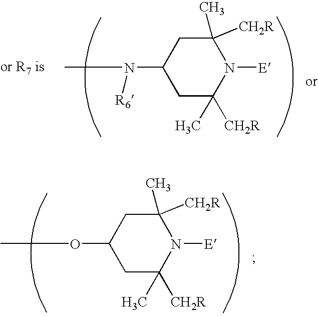
R₈ is defined as for R₇, where one of R₇ and R₈ is —N(R₂)(R₂′);
q is 2 to 8;
X⁻ is an inorganic or organic anion,
Y⁺ is a mono-, di- or tri-valent cation, and
when E is is -O-T-(OH)_{b},
R₂ is glycidyl, C₂-C₁₂alkanoyl substituted by a di(C₁-C₆alkyl) phosphonate, or
R₂ is C₂-C₁₂alkyl, C₂-C₁₂alkanoyl or C₇-C₁₈phenylalkyl, each interrupted by one to six oxygen, sulfur or —N(R₆)— groups; C₁-C₁₂alkyl, C₂-C₁₂alkanoyl, phenyl or C₇-C₁₈phenylalkyl, each substituted by one to six hydroxy groups or by one to six —NHR₆ groups; C₂-C₁₂alkyl, C₂-C₁₂alkanoyl or C₇-C₁₈phenylalkyl, each interrupted by one to three —NR₆C(O)— groups; or R₂ is C₁-C₁₂alkyl, C₂-C₁₂alkanoyl, phenyl or C₇-C₁₈phenylalkyl, each substituted by one to three —SO₃H groups or by one to three —COOR₆ groups; or
R₂ is said alkyl substituted by a piperazine or by a morpholine group; or
R₂ is said interrupted group further substituted by one to six hydroxy groups or by one to six —NHR₆ groups; or
R₂ is said interrupted group further substituted by one to three —SO₃H groups or by one to three —COOR₆ groups; or
R₂ is C₁-C₁₂alkyl, C₂-C₁₂alkanoyl, phenyl or C₇-C₁₈phenylalkyl, each substituted by one or two —COO⁻Y⁺, —N(R₆)(R₆′)⁺X⁻ or —SO₃⁻Y⁺ groups; or
R₂ is said C₁-C₁₂alkyl, C₂-C₁₂alkanoyl, phenyl or C₇-C₁₈phenylalkyl, each of which is substituted by one or two —COO⁻Y⁺, —N(R₆)(R₆′)⁺X⁻ or —SO₃⁻Y⁺ groups, each further substituted by one or two —OH, —COOR₆ or —NHR₆ groups; or
R₂ is a mono-valent homo- or co-oligomer consisting of monomer units derived from monomers selected from the group consisting of ethylene oxide, propylene oxide, ethylene glycol, propylene glycol, acrylic acid, methacrylic acid, ethylene imine, acrylamide, vinyl formamide, vinyl alcohol and vinyl acetate; which homo- or co-oligomer consists of between 2 and about 24 monomer units,
R₂′ is defined as for R₂ and may also be hydrogen,
R₃ is defined as for R₂ and may also be —SO₃H, —PO₃H₂, —SO₃⁻Y⁺ or —PO₃H⁻Y⁺, and
R₄ is defined as for R₂ and may also be hydrogen,
and
when E is alkoxy of 1 to 18 carbon atoms, cycloalkoxy of 5 to 12 carbon atoms or aralkoxy of 7 to 15 carbon atoms,
R₂ is C₁-C₁₂alkyl, C₂-C₁₂alkanoyl, phenyl or C₇-C₁₈phenylalkyl, each substituted by one or two —COO⁻Y⁺, —N(R₆)(R₆′)⁺X⁻ or —SO₃⁻Y⁺ groups; or
R₂ is said C₁-C₁₂alkyl, C₂-C₁₂alkanoyl, phenyl or C₇-C₁₈phenylalkyl, each of which is substituted by one or two —COO⁻Y⁺, —N(R₆)(R₆′)⁺X⁻ or —SO₃⁻Y⁺ groups, each further substituted by one or two —OH, —COOR₆ or —NHR₆ groups, with the proviso that the compound 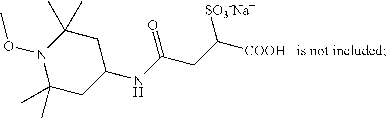
or
R₂ is a mono-valent homo- or co-oligomer consisting of monomer units derived from monomers selected from the group consisting of ethylene oxide, propylene oxide, ethylene glycol, propylene glycol, acrylic acid, methacrylic acid, ethylene imine, acrylamide, vinyl formamide, vinyl alcohol and vinyl acetate; which homo- or co-oligomer consists of between 2 and about 24 monomer units;
R₂′ is defined as for R₂ and may also be hydrogen,
R₃ is defined as for R₂ and may also be —SO₃H, —PO₃H₂, —SO₃⁻Y⁺ or —PO₃H⁻Y⁺, and
R₄ is defined as for R₂ and may also be hydrogen.

2. A compound according to claim 1 where E is -O-T(OH)_{b}.

3. A compound according to claim 1 where E is 2-hydroxycyclohexyloxy or 2-hydroxy-2-methylpropoxy.

4. A compound according to claim 2 of the formula

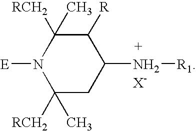

5. A compound according to claim 4 where
R₁ is hydrogen, C₁-C₆alkyl, C₂-C₆alkanoyl, C₂-C₆alkyl or C₂-C₆alkanoyl interrupted by one or two oxygen, sulfur or —N(R₆)— groups; C₁-C₆alkyl or C₂-C₆alkanoyl substituted by one to three hydroxy groups or by one to three —NHR₆ groups, C₂-C₆alkyl or C₂-C₆alkanoyl interrupted by a —NR₆C(O)— group, or is C₁-C₆alkyl or C₂-C₆alkanoyl substituted by a —SO₃H or by a —COOR₆ group.

6. A compound according to claim 4 where
R₁ is hydrogen, C₁-C₄alkyl, C₂-C₅alkanoyl, C₂-C₄alkyl or C₂-C₅alkanoyl interrupted by an oxygen, sulfur or —N(R₆)— group; C₁-C₄alkyl or C₂-C₅alkanoyl substituted by an hydroxy group or by a —NHR₆ group, C₂-C₄alkyl or C₂-C₅alkanoyl interrupted by a —NR₆C(O)— group, or is C₁-C₄alkyl or C₂-C₅alkanoyl substituted by a —SO₃H or by a —COOR₆ group.

7. A compound according to claim 2 of the formula

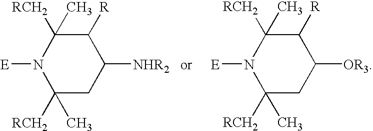

8. A compound according to claim 7 where
R₂ and R₃ are C₂-C₆alkyl or C₂-C₆alkanoyl interrupted by one or two oxygen, sulfur or —N(R₆)— groups; C₁-C₆alkyl or C₂-C₆alkanoyl substituted by one to three hydroxy groups or by one to three —NHR₆ groups, C₂-C₆alkyl or C₂-C₆alkanoyl interrupted by a —NR₆C(O)— group, or R₂ is C₁-C₆alkyl or C₂-C₆alkanoyl substituted by a —SO₃H group or by a —COOR₆ group; or
R₂ and R₃ are C₁-C₆alkyl, C₂-C₆alkanoyl or C₇-C₉phenylalkyl, each substituted by a —COO⁻Y⁺, —N(R₆)(R₆′)⁺X⁻ or —SO₃⁻Y⁺ group.

9. A compound according to claim 7 where
R₂ and R₃ are C₂-C₄alkyl or C₂-C₅alkanoyl interrupted by an oxygen, sulfur or —N(R₆)— group; C₁-C₄alkyl or C₂-C₅alkanoyl substituted by an hydroxy group or by a —NHR₆ group, C₂-C₄alkyl or C₂-C₅alkanoyl interrupted by a —NR₆C(O)— group, or R₂ is C₁-C₄alkyl or C₂-C₅alkanoyl substituted by a —SO₃H group or by a —COOR₆ group; or
R₂ and R₃ are C₁-C₄alkyl or C₂-C₅alkanoyl substituted by a —COO⁻Y⁺, —N(R₆)(R₆′)⁺X⁻ or —SO₃⁻Y⁺ group.

10. A compound according to claim 2 of the formula

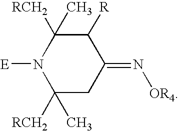

11. A compound according to claim 10 where
R₄ is hydrogen, C₂-C₆alkyl or C₂-C₆alkanoyl interrupted by one or two oxygen, sulfur or —N(R₆)— groups; C₁-C₆alkyl or C₂-C₆alkanoyl substituted by one to three hydroxy groups or by one to three —NHR₆ groups, C₂-C₆alkyl or C₂-C₆alkanoyl interrupted by a —NR₆C(O)— group, or R₄ is C₁-C₆alkyl or C₂-C₆alkanoyl substituted by a —SO₃H group or by a —COOR₆ group; or
R₄ is C₁-C₆alkyl, C₂-C₆alkanoyl or C₇-C₉phenylalkyl, each substituted by a —COO⁻Y⁺, —N(R₆)(R₆′)⁺X⁻ or —SO₃⁻Y⁺ group.

12. A compound according to claim 10 where
R₄ is hydrogen, C₂-C₄alkyl or C₂-C₅alkanoyl interrupted by an oxygen, sulfur or —N(R₆)— group; C₁-C₄alkyl or C₂-C₅alkanoyl substituted by an hydroxy group or by a —NHR₆ group, C₂-C₄alkyl or C₂-C₅alkanoyl interrupted by a —NR₆C(O)— group, or R₄ is C₁-C₄alkyl or C₂-C₅alkanoyl substituted by a —SO₃H group or by a —COOR₆ group; or
R₄ is C₁-C₄alkyl or C₂-C₅alkanoyl substituted by a —COO⁻Y⁺, —N(R₆)(R₆′)⁺X⁻ or —SO₃⁻Y⁺ group.

13. A compound according to claim 2 of the formula

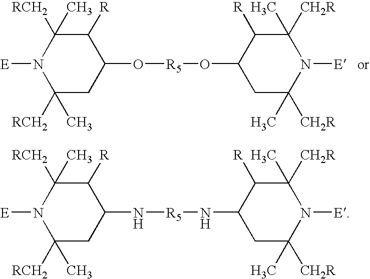

14. A compound according to claim 13 where R₅ is polyethylene glycol or polypropylene glycol.

15. A compound according to claim 2 of the formula 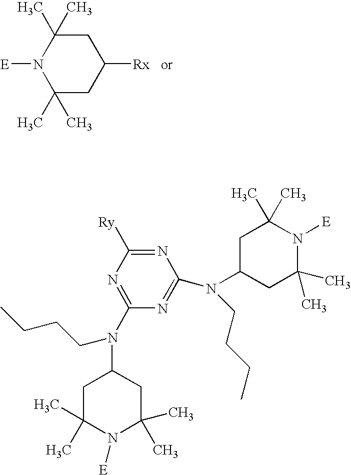
where
E is -O-T(OH)_{b}, and
where Rₓ is selected from the group consisting of
—NH₂⁺CH₂CH₂OH Cl⁻, —NHCH₂CH₂OH, —NH₃⁺⁻OAc, ═NOH, —NHCH(CH₃)COO⁻K⁺, —NHCH₂CH₂N(CH₃)₂⁺⁻OAc, —NHCH₂CH₂SO₃⁻K⁺, —NHCH(COO⁻K⁺)CH₂CH₂SCH₃, —NHCH₂COO⁻K⁺, —NHCOCH₂OH, —NHCOCH₂NHCOCH₃, —NHCH₂CH₂CH₂SO₃H, —OCH₂CH₂OH, —OCH(CH₃)COO⁻K⁺, —OCH₂CH₂N(CH₃)₂⁺⁻OAc, —OCH₂CH₂SO₃⁻K⁺, —OCH(COO⁻K⁺)CH₂CH₂SCH₃, —OCH₂COO⁻K⁺, —OCOCH₂OH, —OCOCH₂NHCOCH₃ and —OCH₂CH₂CH₂SO₃H; and
where R_{y} is selected from the group consisting of
—NHCH₂CH₂NHCH₂CH₂NHCH₂CH₂NH₂, —NH₂⁺CH₂CH₂NHCH₂CH₂NHCH₂CH₂NH₂⁻OAc, —NHPhSO₃H, —NHPhSO₃⁻K⁺, —NHPhSO₃⁻Na⁺, —NH₂⁺PhSO₃H Cl⁻, —NH(3-carboxy-4-chlorophenyl), —NH(3-COO⁻Na⁺-4-chlorophenyl), —NHCH₂CH₂—(N-piperazine), —NH₂⁺CH₂CH₂—(N-piperazine) ⁻OAc and —NH₂⁺CH₂CH₂—(N-piperazine) ⁻Cl.

16. A compound according to claim 1 where E is alkoxy of 1 to 18 carbon atoms, cycloalkoxy of 5 to 12 carbon atoms or aralkoxy of 7 to 15 carbon atoms.

17. A compound according to claim 1 where E is benzyloxy, methoxy, propoxy, butoxy, pentoxy, hexyloxy, heptyloxy, octyloxy or cyclohexyloxy.

18. A compound according to claim 16 of the formula

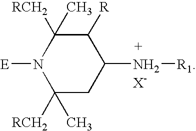

19. A compound according to claim 18 where
R₁ is hydrogen, C₁-C₆alkyl, C₂-C₆alkanoyl, C₂-C₆alkyl or C₂-C₆alkanoyl interrupted by one or two oxygen, sulfur or —N(R₆)— groups; C₁-C₆alkyl or C₂-C₆alkanoyl substituted by one to three hydroxy groups or by one to three —NHR₆ groups, C₂-C₆alkyl or C₂-C₆alkanoyl interrupted by a —NR₆C(O)— group, or is C₁-C₆alkyl or C₂-C₆alkanoyl substituted by a —SO₃H or by a —COOR₆ group.

20. A compound according to claim 18 where
R₁ is hydrogen, C₁-C₄alkyl, C₂-C₅alkanoyl, C₂-C₄alkyl or C₂-C₅alkanoyl interrupted by an oxygen, sulfur or —N(R₆)— group; C₁-C₄alkyl or C₂-C₅alkanoyl substituted by an hydroxy group or by a —NHR₆ group, C₂-C₄alkyl or C₂-C₅alkanoyl interrupted by a —NR₆C(O)— group, or is C₁-C₄alkyl or C₂-C₅alkanoyl substituted by a —SO₃H or by a —COOR₆ group.

21. A compound according to claim 16 of the formula

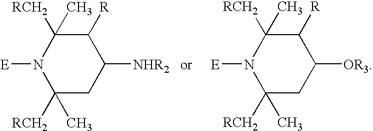

22. A compound according to claim 21 where
R₂ and R₃ are C₁-C₆alkyl, C₂-C₆alkanoyl or C₇-C₉phenylalkyl, each substituted by a —COO⁻Y⁺, —N(R₆)(R₆′)⁺X⁻ or —SO₃⁻Y⁺ group.

23. A compound according to claim 21 where
R₂ and R₃ are C₁-C₄alkyl or C₂-C₅alkanoyl substituted by a —COO⁻Y⁺, —N(R₆)(R₆′)⁺X⁻ or —SO₃⁻Y⁺ group.

24. A compound according to claim 16 of the formula

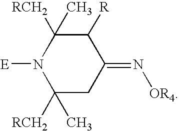

25. A compound according to claim 24 where
R₄ is C₁-C₆alkyl, C₂-C₆alkanoyl or C₇-C₉phenylalkyl, each substituted by a —COO⁻Y⁺, —N(R₆)(R₆′)⁺X⁻ or —SO₃⁻Y⁺ group.

26. A compound according to claim 24 where
R₄ is C₁-C₄alkyl or C₂-C₅alkanoyl substituted by a —COO⁻Y⁺, —N(R₆)(R₆′)⁺X⁻ or —SO₃⁻Y⁺ group.

27. A compound according to claim 16 of the formula.

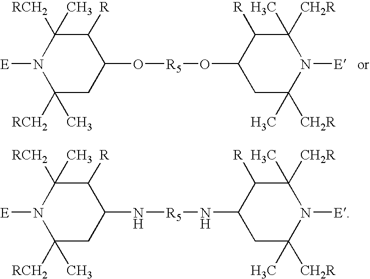

28. A compound according to claim 27 where R₅ is polyethylene glycol or polypropylene glycol.

29. A compound according to claim 16 of the formula 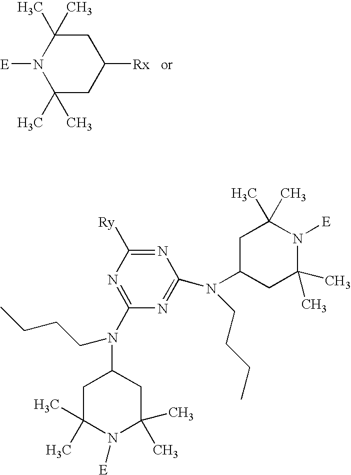
where
E is alkoxy of 1 to 18 carbon atoms, cycloalkoxy of 5 to 12 carbon atoms or aralkoxy of 7 to 15 carbon atoms, and
Rₓ is selected from the group consisting of
—NH₂⁺CH₂CH₂OH Cl⁻, —NH₃⁺⁻OAc, ═NOH, —NHCH(CH₃)COO⁻K⁺, —NHCH₂CH₂N(CH₃)₂⁺⁻OAc, —NHCH₂CH₂SO₃⁻K⁺, —NHCH(COO⁻K⁺)CH₂CH₂SCH₃, —NHCH₂COO⁻K⁺, —OCH(CH₃)COO⁻K⁺, —OCH₂CH₂N(CH₃)₂⁺⁻OAc, —OCH₂CH₂SO₃⁻K⁺, —OCH(COO⁻K⁺)CH₂CH₂SCH₃ and —OCH₂COO⁻K⁺ and
where R_{y} is selected from the group consisting of
—NHCH₂CH₂NHCH₂CH₂NHCH₂CH₂NH₂, —NH₂⁺CH₂CH₂NHCH₂CH₂NHCH₂CH₂NH₂⁻OAc, —NHPhSO₃H, —NHPhSO₃⁻K⁺, —NHPhSO₃⁻Na⁺, —NH₂⁺PhSO₃H Cl⁻, —NH(3-carboxy-4-chlorophenyl), —NH(3-COO⁻Na⁺-4-chlorophenyl), —NHCH₂CH₂—(N-piperazine), —NH₂⁺CH₂CH₂—(N-piperazine) ⁻OAc and —NH₂⁺CH₂CH₂—(N-piperazine) ⁻Cl.

30. A stabilized composition comprising
an organic material subject to the deleterious effects of light, heat and oxygen, and
an effective stabilizing amount of a water compatible or water soluble sterically hindered alkoxyamine or hydroxy substituted alkoxyamine compound according to claim 1.

31. A stabilized composition comprising
an organic material subject to the deleterious effects of light, heat and oxygen, and
an effective stabilizing amount of a water compatible or water soluble sterically hindered alkoxyamine or hydroxy substituted alkoxyamine compound according to claim 2.

32. A stabilized composition comprising
an organic material subject to the deleterious effects of light, heat and oxygen, and
an effective stabilizing amount of a water compatible or water soluble sterically hindered alkoxyamine or hydroxy substituted alkoxyamine compound according to claim 16.

33. A composition according to claim 30 which is a coating, ink jet ink, ink jet recording material, photographic recording material, multi-layer polymer structure, a coextruded film, a radiation cured film, ink or coating; an adhesive or a laminate.

34. A composition according to claim 30 which additionally comprises an effective stabilizing amount of at least one coadditive stabilizer selected from the group consisting of the phenolic antioxidants, metal stearates, metal oxides, organophosphorus compounds, furanone antioxidants, hydroxylamines, ultraviolet light absorbers, and other hindered amine light stabilizers.

35. A composition according to claim 30 which additionally comprises an ultraviolet light absorber selected from the group consisting of the benzophenones, 2H-benzotriazoles, aryl-s-triazines.

36. A composition according to claim 30 which is a colored composition containing pigments or dyes.

37. A composition according to claim 30 which is a colored composition containing dyes.

38. A composition according to claim 30 which is a colored composition containing dyes, which composition is selected from the group consisting of ink jet inks, ink jet recording media, coatings, body care products, household products, textiles and fabrics.
